(19) United States
(12) Patent Application Publication
(10) Pub. No.: US 2023/0146795 A1
(43) Pub. Date: May 11, 2023

(54) ENHANCEMENT OF ANTI-TUMOR ACTIVITY OF SHP2 INHIBITOR PYRIMIDINONE IN COMBINATION WITH NOVEL CANCER MEDICINES IN CANCERS

(71) Applicants: TAIHO PHARMACEUTICAL CO., LTD., Tokyo (JP); OTSUKA PHARMACEUTICAL CO., LTD., Tokyo (JP)

(72) Inventors: Takuya HOSHINO, Tsukuba-shi, Ibaraki (JP); Yu KOMIYA, Tsukuba-shi, Ibaraki (JP); Yoko NAKATSURU, Tsukuba-shi, Ibaraki (JP); Nicola Gail WALLIS, Cambridge (GB)

(73) Assignees: TAIHO PHARMACEUTICAL CO., LTD., Tokyo (JP); OTSUKA PHARMACEUTICAL CO., LTD., Tokyo (JP)

(21) Appl. No.: 17/759,335

(22) PCT Filed: Jan. 22, 2021
(86) PCT No.: PCT/JP2021/002318

§ 371 Date: Jul. 22, 2022

(30) Foreign Application Priority Data
Jan. 24, 2020 (JP) 2020-010300
Oct. 5, 2020 (JP) 2020-168593

(51) Int. Cl. A61K 31/519; A61K 45/06; A61P 35/00

(52) U.S. Cl. CPC A61K 31/519; A61K 45/06; A61P 35/00

(57) ABSTRACT

The present invention provides a combination drug for the treatment of a malignant tumor comprising 2-((1R,2R,4S)-2-amino-7-azabicyclo[2.2.1]heptan-7-yl)-5-(3,4-dichloro-2-methyl-2H-indazol-5-yl)-3-methyl-3,7-dihydro-4H-pyrrolo[2,3-d]pyrimidin-4-one or a pharmaceutically acceptable salt thereof, and at least one additional compound having an antitumor effect or at least one pharmaceutically acceptable salt thereof, and a pharmaceutical composition comprising both the active ingredients.

### TECHNICAL FIELD

The present invention relates to a combination drug for the treatment of a malignant tumor by concomitant use of a specific SHP2 inhibitor and an antitumor agent, an antitumor effect enhancer, an antitumor agent, use of a compound in the enhancement of the antitumor effect of at least one additional compound, a method for treating a tumor, and a kit for a malignant tumor treatment.

### CROSS-REFERENCE TO RELATED PATENT APPLICATIONS

The present application claims priority to Japanese Patent Application No. 2020-010300 filed on Jan. 24, 2020 and Japanese Patent Application No. 2020-168593 filed on Oct. 5, 2020, which are incorporated herein by reference in their entirety.

### BACKGROUND ART

Src homology region 2 domain-containing phosphatase-2 (SHP-2) is a ubiquitously expressed protein tyrosine phosphatase encoded by the PTPN11 gene. SHP2 contains two N-terminal tandem SH2 domains (N—SH₂, C—SH₂), a catalytic protein tyrosine phosphatase (PTP) domain and a C-terminal tail with 2 tyrosine phosphorylation sites.

SHP2 switches between “open” active and “closed” inactive forms due to autoinhibitory interactions between the N—SH₂ and the PTP domain. This naturally occurring autoinhibition is released when bis-tyrosylphosphorylated peptides bind to the N—SH₂ domains and SHP2 adopts an “open” conformation, resulting in activation of the enzyme and exposure of the PTP domain for substrate recognition and catalysis.

PTPN11 mutations have been linked to several human diseases including cancer. Germline PTPN11 mutations are associated with developmental disorders such as Noonan Syndrome and Leopard Syndrome, whilst somatic mutations occur in several types of hematologic malignancies, such as JIMML and more rarely in solid tumors.

SHP2 is required for signaling downstream of receptor tyrosine kinases (e.g. EGFR, ALK, PDGFR) and plays a positive role in regulating many cellular processes such as proliferation in response to growth factor and cytokine stimulation. Previous studies have shown that SHP2 acts upstream of Ras and is required for full, sustained activation of the MAPK pathway. RTK deregulation often leads to a wide range of cancers, making SHP2 a valuable target in RTK-activated cancers.

It has been reported that some SHP2 inhibitor compounds show inhibitory effect on proliferation of in vitro cancer cells and on increase in tumor volume in a mouse xenograft model (Nature (2016) 535: 148-152). As medicines for use in chemotherapy, molecular targeting drugs and cytotoxic drugs have been developed.

### CITATION LIST

### Non Patent Literature

[NPL 1]
Nature (2016) 535: 148-152

### SUMMARY OF INVENTION

### Technical Problem

Even antitumor agents having high therapeutic effects need to be carefully used or may be unable to be used in some cases, if these agents have severe side effects or are highly toxic. It is also known that such antitumor agents may differ in effect among patients or may reduce their effects due to the long-term administration of the same agent.

An object of the present invention is to provide a novel combination drug for the treatment of a malignant tumor, a novel antitumor effect enhancer, a novel antitumor agent, comprising an SHP2 inhibitor that exhibits a remarkably excellent antitumor effect and has fewer side effects.

An object of the present invention is to provide novel use of an SHP2 inhibitor that exhibits a remarkably excellent antitumor effect and has fewer side effects in the enhancement of the antitumor effect of at least one additional compound having an antitumor effect.

An object of the present invention is to provide a novel method for treating a tumor comprising administering an SHP2 inhibitor that exhibits a remarkably excellent antitumor effect and has fewer side effects.

An object of the present invention is to provide a kit for malignant tumor treatment comprising an SHP2 inhibitor that exhibits a remarkably excellent antitumor effect and has fewer side effects.

### Solution to Problem

The present inventors conducted extensive research to achieve the above objects, and consequently found that a combination drug comprising a compound represented by formula (I), which is a potent SHP2 inhibitor, or a tautomer thereof, a solvate thereof, or a pharmaceutically acceptable salt thereof, and at least one additional compound having an antitumor effect, at least one tautomer thereof, at least one solvate thereof, or at least one pharmaceutically acceptable salt thereof selected from the group consisting of molecular targeted drugs and cytotoxic drugs exhibits a remarkably excellent antitumor effect and has fewer side effects.

The present invention comprises the following items.

### Item 1

A combination drug for the treatment of a malignant tumor comprising

a compound represented by formula (I):

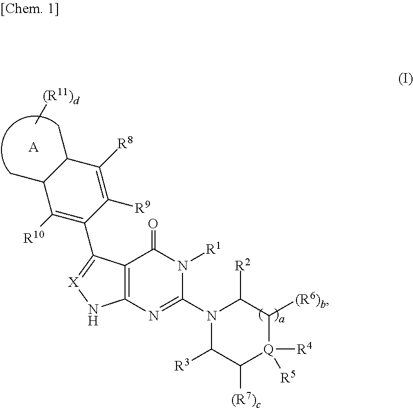

or a tautomer thereof, a solvate thereof, or a pharmaceutically acceptable salt thereof, wherein:

### X is CH or N;

R¹ is —CH₃;
R² and R³ are independently selected from the group consisting of hydrogen and C₁₋₄alkyl;

### Q is C or N;

wherein when Q is C, then either:
   (i) R⁴ is amino, aminoC₁₋₄alkyl, or monoC₁₋₄alkylamino; R⁵ is hydrogen, C₁₋₄alkyl, halogen, hydroxyC₁₋₄alkyl, C₁₋₄alkoxy, haloC₁₋₄alkyl, or C₁₋₄alkoxyC₁₋₄alkyl;
   or
   (ii) R⁴ and R⁵ together with Q form a four- to six-membered ring that can optionally contain 1 to 3 heteroatoms or groups independently selected from the group consisting of N, O, S, NH, C(O), and S(O)ₘ, and said ring formed by R⁴ and R³ can be unsubstituted or substituted with 1 to 4 groups independently selected from the group consisting of amino, halogen, haloC₁₋₄alkyl, hydroxyl, methoxy, methylamino, and C₁₋₄alkyl, and m is selected from the group consisting of 1 and 2; and
wherein when Q is N, then:
   R⁴ is absent; and
   R⁵ is hydrogen;
      R⁶ and R⁷ are independently selected from the group consisting of halogen, C₁₋₄alkyl, hydroxyC₁₋₄alkyl, and hydroxyl, provided that when Q is N, then R⁶ or R⁷ is not halogen or hydroxyl;
      or any two groups selected from the group consisting of R², R³, R⁶, and R⁷ together form a one- to three-membered bridge group selected from the group consisting of C₁₋₃alkylene, C₂₋₃alkenylene, methylene-NR^{q}-methylene, and methylene-O-methylene, wherein the bridge group is optionally substituted with a group selected from the group consisting of C₁₋₄alkyl, hydroxyl, and halogen, and R^{q} is selected from the group consisting of hydrogen and C₁₋₄alkyl;
      or R⁴ and R⁷ form a four- to six-membered ring containing a N atom;
      or R³ and R⁷ form a three- to six-membered ring;
      or R⁶ and R⁷ form a direct bond;
      a is selected from the group consisting of 0, 1, and 2;
      b is selected from the group consisting of 0, 1, and 2;
      c is selected from the group consisting of 0, 1, and 2;
      or Q is C, c is 2, R⁴ is hydrogen, and the two R⁷ join to form a 4 to 6-membered nitrogen containing ring;
      Ring A is either:
(i) a five-membered nitrogen-containing heterocyclic ring wherein the heterocyclic ring optionally contains one or two additional heteroatoms selected from the group consisting of N, O, and S, or
(ii) a six-membered aromatic nitrogen-containing heterocyclic ring, wherein the heterocyclic ring optionally contains one or two additional heteroatoms selected from the group consisting of N, O, and S; or
(iii) a six-membered non-aromatic nitrogen-containing heterocyclic ring, wherein the heterocyclic ring optionally contains one or two additional heteroatoms selected from the group consisting of N and S;
   R⁸ is selected from the group consisting of hydrogen, C₁₋₄alkyl, haloC₁₋₄alkyl, and halogen;
   R⁹ is selected from the group consisting of hydrogen and halogen;
   R¹⁰ is selected from the group consisting of haloC₁₋₄alkyl, C₁₋₄alkyl, halogen, hydrogen, and C₁₋₄alkoxy;
   R¹¹ are independently selected from the group consisting of halogen, cyano, cyanoC₁₋₄alkyl, hydroxyl, oxo (═O), C₁₋₄alkyl optionally substituted with a five- or six-membered heterocyclic group containing 1 or 2 heteroatoms selected from the group consisting of O, N, and S, haloC₁₋₄alkyl, C₁₋₄alkoxy, hydroxylC₁₋₄alkyl, C₁₋₄alkoxyC₁₋₄alkyl, C₁₋₄alkylsulfone, amino, monoC₁₋₄alkylamino, diC₁₋₄alkylamino, aminoC₁₋₄alkyl, —C₁₋₄alkylene-C(═O)NH_{(2-q)}(C₁₋₆alkyl)_{q}), —C₁₋₄alkylene-NHC(═O)C₁₋₆alkyl, sulfonamide, sulfonamideC₁₋₄alkyl, 3 to 6-membered cycloalkyl, C₁₋₄alkyl substituted with 3 to 6-membered cycloalkyl, a five- or six-membered unsaturated heterocyclic group containing 1, 2, 3 or 4 heteroatoms selected from the group consisting of 0, N, and S, and an optionally substituted four- to six-membered saturated heterocyclic group containing 1 or 2 heteroatoms selected from the group consisting of O, N, and S where the optional substituent is selected from C₁₋₄alkyl;
   q is selected from the group consisting of 0, 1, and 2; and
   d is selected from the group consisting of 0, 1, and 2, and

at least one additional compound having an antitumor effect, at least one tautomer thereof, at least one solvate thereof, or at least one pharmaceutically acceptable salt thereof, selected from the group consisting of molecular targeted drugs and cytotoxic drugs.

### Item 2

The combination drug according to item 1, wherein the compound represented by formula (I), the tautomer thereof, the solvate thereof, or the pharmaceutically acceptable salt thereof is administered before, simultaneously with, or after administration of the at least one additional compound having an antitumor effect, the at least one tautomer thereof, the at least one solvate thereof, or the at least one pharmaceutically acceptable salt thereof.

### Item 3

The combination drug according to item 1 or 2, wherein the compound represented by formula (I) is selected from the group consisting of 2-((1R,2R,4S)-2-amino-7-azabicyclo[2.2.1]heptan-7-yl)-5-(3,4-dichloro-2-methyl-2H-indazol-5-yl)-3-methyl-3,7-dihydro-4H-pyrrolo[2,3-d]pyrimidin-4-one, 6-((1R,4R,7R)-7-amino-2-azabicyclo[2.2.1]heptan-2-yl)-3-(3,4-dichloro-2-methyl-2H-indazol-5-yl)-5-methyl-1,5-dihydro-4H-pyrazolo[3,4-d]pyrimidin-4-one, 2-((1R,4R,7R)-7-amino-2-azabicyclo[2.2.1]heptan-2-yl)-5-(4-chloro-2-ethyl-2H-indazol-5-yl)-3-methyl-3,7-dihydro-4H-pyrrolo[2,3-d]pyrimidin-4-one, 2-((1R,4R,7R)-7-amino-2-azabicyclo[2.2.1]heptan-2-yl)-5-(4-chloro-2-methyl-2H-indazol-5-yl)-3-methyl-3,7-dihydro-4H-pyrrolo[2,3-d]pyrimidin-4-one, 2-((1R,4R,7R)-7-amino-2-azabicyclo[2.2.1]heptan-2-yl)-5-(3,4-dichloro-2-methyl-2H-indazol-5-yl)-3-methyl-3,7-dihydro-4H-pyrrolo[2,3-d]pyrimidin-4-one, 2-((1R,2R,4S)-2-amino-7-azabicyclo[2.2.1]heptan-7-yl)-5-(3-chloro-4-fluoro-2-methyl-2H-indazol-5-yl)-3-methyl-3,7-dihydro-4H-pyrrolo[2,3-d]pyrimidin-4-one, and 2-((1R,2R,4S)-2-amino-7-azabicyclo[2.2.1]heptan-7-yl)-5-(4-chloro-2-ethyl-2H-indazol-5-yl)-3-methyl-3,7-dihydro-4H-pyrrolo[2,3-d]pyrimidin-4-one.

### Item 4

The combination drug according to any one of items 1 to 3, wherein the compound represented by formula (I) is 2-((1R,2R,4S)-2-amino-7-azabicyclo[2.2.1]heptan-7-yl)-5-(3,4-dichloro-2-methyl-2H-indazol-5-yl)-3-methyl-3,7-dihydro-4H-pyrrolo[2,3-d]pyrimidin-4-one.

### Item 5

The combination drug according to any one of items 1 to 4, wherein the at least one additional compound having an antitumor effect, the at least one tautomer thereof, the at least one solvate thereof, or the at least one pharmaceutically acceptable salt thereof is selected from the group consisting of a tyrosine kinase inhibitor, a RAS-MAPK pathway inhibitor, a PI3K pathway inhibitor, a BCL2 inhibitor, a CDK4/6 inhibitor, an HDAC inhibitor, a topoisomerase inhibitor (a topoisomerase I inhibitor, and topoisomerase II inhibitor), an alkylating agent, an anthracycline antibiotic, an alkaloid, an anti-metabolite, an anti-microtubule agent, a platinum-containing drug, proteasome inhibitor, and a thalidomide analog drug.

### Item 6

The combination drug according to any one of items 1 to 5, wherein the molecular targeted drug is selected from the group consisting of an AKT inhibitor, ALK inhibitor, Bcl2 inhibitor, BCR-ABL inhibitor, BRAF inhibitor, CDK4/6 inhibitor, CDK inhibitor, EGFR inhibitor, Erk1/2 inhibitor, FGFR inhibitor, FLT3 inhibitor, HER2 inhibitor, MEK inhibitor, Multi-kinase inhibitor, PI3K inhibitor, RAF inhibitor, HDAC inhibitor, topoisomerase I inhibitor, topoisomerase II inhibitor, alkylating agent, anthracycline antibiotics, alkaloid, anti-metabolite, anti-microtubule agent, platinum-containing drug, and proteasome inhibitor.

### Item 7

The combination drug according to any one of items 1 to 6, wherein the molecular targeted drug is selected from the group consisting of cetuximab, MK-2206, alectinib, crizotinib, venetoclax, imatinib, dasatinib, ponatinib, dabrafenib, vemurafenib, sorafenib, palbociclib, abemaciclib, osimertinib, gefitinib, erlotinib, afatinib, brigatinib, ulixertinib, (2R)-2-(6-{5-chloro-2-[(oxan-4-yl)amino]pyrimidin-4-yl}-1-oxo-2,3-dihydro-1H-isoindol-2-yl)-N-[(1S)-1-(3-fluoro-5-methoxyphenyl)-2-hydroxyethyl]propanamide or a salt thereof, erdafitinib, giltertinib, lapatinib, neratinib, trametinib, cobimetinib, binimetinib, regorafenib, sunitinib, nintedanib, anlotinib, vandetanib, lenvatinib, alpelisib and idelalisib/CAL-101, vorinostat (SAHA), irinotecan (SN-38), etoposide, cyclophosphamide, doxorubicin, gemcitabine, pemetrexed, 5-FU, FdUrd, FTD, paclitaxel, cisplatin, oxaliplatin, and bortezomib, afuresertib, trans-3-amino-1-methyl-3-(4-(3-phenyl-5H-imidazo[1,2-c]pyrido[3,4-e][1,3]oxazin-2-yl)phenyl)cyclobutanol, capivasertib, ipatasertib, triciribine, miransertib, lorlatinib, ceritinib, repotrectinib, ensartinib, alkotinib, WX-0593, SAF-189s, CT-707, TQ-B3101, sabutoclax, apogossypol, obatoclax, navitoclax, APG-2575, APG-1252, asciminib, olverembatinib, encorafenib, lifirafenib, LXH-254, ribociclib, lerociclib, trilaciclib, alvocidib, GLR-2007, SHR-6390, XZP-3287, BPI-1178, PF-06873600, NUV-422, FCN-437, seliciclib, mevociclib, milciclib, fadraciclib, zotiraciclib, dinaciclib, samuraciclib, voruciclib, FIT-039, PF-07104091, BEY-1107, panitumumab, sutetinib, allitinib, epitinib, xiliertinib, rociletinib, dacomitinib, simotinib, olmutinib, yinlitinib, mefatinib, alflutinib, almonertinib, icotinib, naquotinib, poziotinib, epertinib, sapitinib, cipatinib, tarloxotinib, pyrotinib, pirotinib, lazertinib, varlitinib, tesevatinib, canertinib, mobocertinib, duligotuzumab, olafertinib, zorifertinib, pelitinib, DZD-9008, ASK-120067, BPI-7711, QLNC-120, ametumumab, imgatuzumab, amivantamab, seribantumab, nimotuzumab, serclutamab, depatuxizumab, tomuzotuximab, SCT-200, ravoxertinib, LY3214996, MK-8353, LTT462, HH-2710, infigratinib, pemigatinib, orantinib, derazantinib, roblitinib, rogaratinib, zoligratinib, E-7090, AZD-4547, ODM-203, ICP-192, HMPL-453, bemarituzumab, quizartinib, crenolanib, flysyn, mivavotinib, PHI-101, MEN-1703, FF-10101, HM-43239, E-6201, ENMD-2076, larotinib, tucatinib (irbinitinib), BDTX-189, trastuzumab, pertuzumab, zanidatamab, zenocutuzumab, margetuximab, KN-026, BAT-8001, TAA-013, KL-A166, selumetinib, refametinib, mirdametinib, pimasertib, HL-085, NFX-179, nilotinib, dovitinib, axitinib, vatalinib, pazopanib, avapritinib, famitinib, catequentinib, necuparanib, surufatinib, lucitanib, midostaurin, vorolanib, bevasiranib, bevacizumab, ranibizumab, vanucizumab, navicixizumab, ramucirumab, BAT-5906, VGX-100, CSL-346, duvelisib, copanlisib, buparlisib, paxalisib, voxtalisib, zandelisib, dezapelisib, linperlisib, inavolisib, parsaclisib, eganelisib, nemiralisib, seletalisib, gedatolisib, leniolisib, tenalisib, pictilisib, bimiralisib, BBP-681, BGB-10188, MEN-1611, ASN-003, ACP-319, panobinostat, resminostat, abexinostat, romidepsin, belinostat, entinostat, quisinostat, pracinostat, tefinostat, mocetinostat, givinostat, dacinostat, ivaltinostat, domatinostat, fimepinostat, tinostamustine, Remetinostat, tucidinostat, ricolinostat, CXD-101, REC-2282, veltuzumab, rituximab, ublituximab, nogitecan, simmitecan, gimatecan, topotecan, cositecan, belotecan, govitecan, deruxtecan, AR-67, camsirubicin, Aldoxorubicin, vosaroxin, mitoxantrone, evofosfamide, amrubicin, sobuzoxane, epirubicin, F-14512, dacarbazine, temozolomide, nimustine, busulfan, procarbazine, melphalan, mitomycin C, daunorubicin, vincristine, vinblastine, vinorelbine, eribulin, trifluridine, docetaxel, cabazitaxel, avanbulin, fluorapacin, mertansine, carboplatin, nedaplatin, ixazomib, marizomib, carfilzomib and LXE-408.

### Item 8

The combination drug according to any one of items 1 to 7, wherein the malignant tumor is selected from the group consisting of epithelial cancer (respiratory cancer, gastrointestinal cancer, genital cancer, cancer of the secretory system, breast cancer, etc.), mesothelioma, sarcoma, hematopoietic tumor, tumors of the central nervous system, retinoblastoma, and tumors of the peripheral nervous system.

### Item 9

An antitumor effect enhancer for at least one additional compound having an antitumor effect, at least one tautomer thereof, at least one solvate thereof, or at least one pharmaceutically acceptable salt thereof, selected from the group consisting of molecular targeted drugs and cytotoxic drugs,

the antitumor effect enhancer comprising a compound represented by formula (I)

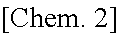

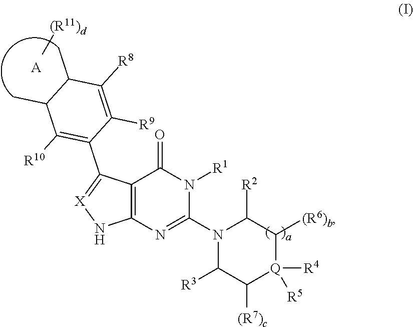

or a tautomer thereof, a solvate thereof, or a pharmaceutically acceptable salt thereof, wherein:

### X is CH or N;

R¹ is —CH₃;
R² and R³ are independently selected from the group consisting of hydrogen and C₁₋₄alkyl;

### Q is C or N;

wherein when Q is C then either:
   (i) R⁴ is amino, aminoC₁₋₄alkyl, or monoC₁₋₄alkylamino; R⁵ is hydrogen, C₁₋₄alkyl, halogen, hydroxyC₁₋₄alkyl, C₁₋₄alkoxy, haloC₁₋₄alkyl, or C₁₋₄alkoxyC₁₋₄alkyl;
   or
   (ii) R⁴ and R⁵ together with Q form a four- to six-membered ring that can optionally contain 1 to 3 heteroatoms or groups independently selected from the group consisting of N, O, S, NH, C(O), and S(O)ₘ, and said ring formed by R⁴ and R³ can be unsubstituted or substituted with 1 to 4 groups independently selected from the group consisting of amino, halogen, haloC₁₋₄alkyl, hydroxyl, methoxy, methylamino, and C₁₋₄alkyl, and m is selected from the group consisting of 1 and 2; and
wherein when Q is N then:
   R⁴ is absent; and
   R⁵ is hydrogen;
      R⁶ and R⁷ are independently selected from the group consisting of halogen, C₁₋₄alkyl, hydroxyC₁₋₄alkyl, and hydroxyl, provided that when Q is N, then R⁶ or R⁷ is not halogen or hydroxyl;
      or any two groups selected from the group consisting of R², R³, R⁶, and R⁷ together form a one- to three-membered bridge group selected from the group consisting of C₁₋₃alkylene, C₂₋₃alkenylene, methylene-NR^{q}-methylene, and methylene-O-methylene, wherein the bridge group is optionally substituted with a group selected from the group consisting of C₁₋₄alkyl, hydroxyl, and halogen, and R^{q} is selected from the group consisting of hydrogen and C₁₋₄alkyl;
      or R⁴ and R⁷ form a four- to six-membered ring containing a N atom;
      or R³ and R⁷ form a three- to six-membered ring;
      or R⁶ and R⁷ form a direct bond;
      a is selected from the group consisting of 0, 1, and 2;
      b is selected from the group consisting of 0, 1, and 2;
      c is selected from the group consisting of 0, 1, and 2;
      or Q is C, c is 2, R⁴ is hydrogen, and the two R⁷ join to form a 4 to 6-membered nitrogen containing ring;
      Ring A is either:
(i) a five-membered nitrogen-containing heterocyclic ring wherein the heterocyclic ring optionally contains one or two additional heteroatoms selected from the group consisting of N, O, and S, or
(ii) a six-membered aromatic nitrogen-containing heterocyclic ring, wherein the heterocyclic ring optionally contains one or two additional heteroatoms selected from the group consisting of N, O, and S; or
(iii) a six-membered non-aromatic nitrogen-containing heterocyclic ring, wherein the heterocyclic ring optionally contains one or two additional heteroatoms selected from the group consisting of N and S;
   R⁸ is selected from the group consisting of hydrogen, C₁₋₄alkyl, haloC₁₋₄alkyl, and halogen;
   R⁹ is selected from the group consisting of hydrogen and halogen;
   R¹⁰ is selected from the group consisting of haloC₁₋₄alkyl, C₁₋₄alkyl, halogen, hydrogen, and C₁₋₄alkoxy;
   R¹¹ are independently selected from the group consisting of halogen, cyano, cyanoC₁₋₄alkyl, hydroxyl, oxo (═O), C₁₋₄alkyl optionally substituted with a five- or six-membered heterocyclic group containing 1 or 2 heteroatoms selected from the group consisting of O, N, and S, haloC₁₋₄alkyl, C₁₋₄alkoxy, hydroxylC₁₋₄alkyl, C₁₋₄alkoxyC₁₋₄alkyl, C₁₋₄alkylsulfone, amino, monoC₁₋₄alkylamino, diC₁₋₄alkylamino, aminoC₁₋₄alkyl, —C₁₋₄alkylene-C(═O)NH_{(2-q)}(C₁₋₆alkyl)_{q}), —C₁₋₄alkylene-NHC(═O)C₁₋₆alkyl, sulfonamide, sulfonamideC₁₋₄alkyl, 3 to 6-membered cycloalkyl, C₁₋₄alkyl substituted with 3 to 6-membered cycloalkyl, a five- or six-membered unsaturated heterocyclic group containing 1, 2, 3 or 4 heteroatoms selected from the group consisting of 0, N, and S, and an optionally substituted four- to six-membered saturated heterocyclic group containing 1 or 2 heteroatoms selected from the group consisting of O, N, and S where the optional substituent is selected from C₁₋₄alkyl;
   q is selected from the group consisting of 0, 1, and 2; and
   d is selected from the group consisting of 0, 1, and 2 as an active ingredient.

### Item 10

The antitumor effect enhancer according to item 9, wherein the compound represented by formula (I), the tautomer thereof, the solvate thereof, or the pharmaceutically acceptable salt thereof is administered before, simultaneously with, or after administration of the at least one additional compound having an antitumor effect, the at least one tautomer thereof, the at least one solvate thereof, or the at least one pharmaceutically acceptable salt thereof.

### Item 11

The antitumor effect enhancer according to item 9 or 10, wherein the compound represented by formula (I) is selected from the group consisting of 2-((1R,2R,4S)-2-amino-7-azabicyclo[2.2.1]heptan-7-yl)-5-(3,4-dichloro-2-methyl-2H-indazol-5-yl)-3-methyl-3,7-dihydro-4H-pyrrolo[2,3-d]pyrimidin-4-one, 6-((1R,4R,7R)-7-amino-2-azabicyclo[2.2.1]heptan-2-yl)-3-(3,4-dichloro-2-methyl-2H-indazol-5-yl)-5-methyl-1,5-dihydro-4H-pyrazolo[3,4-d]pyrimidin-4-one, 2-((1R,4R,7R)-7-amino-2-azabicyclo[2.2.1]heptan-2-yl)-5-(4-chloro-2-ethyl-2H-indazol-5-yl)-3-methyl-3,7-dihydro-4H-pyrrolo[2,3-d]pyrimidin-4-one, 2-((1R,4R,7R)-7-amino-2-azabicyclo[2.2.1]heptan-2-yl)-5-(4-chloro-2-methyl-2H-indazol-5-yl)-3-methyl-3,7-dihydro-4H-pyrrolo[2,3-d]pyrimidin-4-one, 2-((1R,4R,7R)-7-amino-2-azabicyclo[2.2.1]heptan-2-yl)-5-(3,4-dichloro-2-methyl-2H-indazol-5-yl)-3-methyl-3,7-dihydro-4H-pyrrolo[2,3-d]pyrimidin-4-one, 2-((1R,2R,4S)-2-amino-7-azabicyclo[2.2.1]heptan-7-yl)-5-(3-chloro-4-fluoro-2-methyl-2H-indazol-5-yl)-3-methyl-3,7-dihydro-4H-pyrrolo[2,3-d]pyrimidin-4-one, and 2-((1R,2R,4S)-2-amino-7-azabicyclo[2.2.1]heptan-7-yl)-5-(4-chloro-2-ethyl-2H-indazol-5-yl)-3-methyl-3,7-dihydro-4H-pyrrolo[2,3-d]pyrimidin-4-one.

### Item 12

The antitumor effect enhancer according to any one of items 9 to 11, wherein the compound represented by formula (I) is 2-((1R,2R,4S)-2-amino-7-azabicyclo[2.2.1]heptan-7-yl)-5-(3,4-dichloro-2-methyl-2H-indazol-5-yl)-3-methyl-3,7-dihydro-4H-pyrrolo[2,3-d]pyrimidin-4-one.

### Item 13

The antitumor effect enhancer according to any one of items 9 to 12, wherein the at least one additional compound having an antitumor effect, the at least one tautomer thereof, the at least one solvate thereof, or the at least one pharmaceutically acceptable salt thereof is selected from the group consisting of a tyrosine kinase inhibitor, a RAS-MAPK pathway inhibitor, a PI3K pathway inhibitor, a BCL2 inhibitor, a CDK4/6 inhibitor, an HDAC inhibitor, a topoisomerase inhibitor (a topoisomerase I inhibitor, and topoisomerase II inhibitor), an alkylating agent, an anthracycline antibiotic, an alkaloid, an anti-metabolite, an anti-microtubule agent, a platinum-containing drug, a proteasome inhibitor, and a thalidomide analog drug.

### Item 14

The antitumor effect enhancer according to any one of items 9 to 13, wherein the molecular targeted drug is selected from the group consisting of an AKT inhibitor, ALK inhibitor, Bcl2 inhibitor, BCR-ABL inhibitor, BRAF inhibitor, CDK4/6 inhibitor, CDK inhibitor, EGFR inhibitor, Erk1/2 inhibitor, FGFR inhibitor, FLT3 inhibitor, HER2 inhibitor, MEK inhibitor, Multi-kinase inhibitor, PI3K inhibitor, RAF inhibitor, HDAC inhibitor, topoisomerase I inhibitor, topoisomerase II inhibitor, alkylating agent, anthracycline antibiotics, alkaloid, anti-metabolite, anti-microtubule agent, platinum-containing drug, and proteasome inhibitor.

### Item 15

The antitumor effect enhancer according to any one of items 9 to 14, wherein the molecular targeted drug is selected from the group consisting of cetuximab, MK-2206, alectinib, crizotinib, venetoclax, imatinib, dasatinib, ponatinib, dabrafenib, vemurafenib, sorafenib, palbociclib, abemaciclib, osimertinib, gefitinib, erlotinib, afatinib, brigatinib, ulixertinib, (2R)-2-(6-{5-chloro-2-[(oxan-4-yl)amino]pyrimidin-4-yl}-1-oxo-2,3-dihydro-1H-isoindol-2-yl)-N-[(1S)-1-(3-fluoro-5-methoxyphenyl)-2-hydroxyethyl]propanamide or a salt thereof, erdafitinib, giltertinib, lapatinib, neratinib, trametinib, cobimetinib, binimetinib, regorafenib, sunitinib, nintedanib, anlotinib, vandetanib, lenvatinib, alpelisib and idelalisib/CAL-101, vorinostat (SAHA), irinotecan (SN-38), etoposide, cyclophosphamide, doxorubicin, gemcitabine, pemetrexed, 5-FU, FdUrd, FTD, paclitaxel, cisplatin, oxaliplatin, bortezomib, afuresertib, trans-3-amino-1-methyl-3-(4-(3-phenyl-5H-imidazo[1,2-c]pyrido[3,4-e][1,3]oxazin-2-yl)phenyl)cyclobutanol, capivasertib, ipatasertib, triciribine, miransertib, lorlatinib, ceritinib, repotrectinib, ensartinib, alkotinib, WX-0593, SAF-189s, CT-707, TQ-B3101, sabutoclax, apogossypol, obatoclax, navitoclax, APG-2575, APG-1252, asciminib, olverembatinib, encorafenib, lifirafenib, LXH-254, ribociclib, lerociclib, trilaciclib, alvocidib, GLR-2007, SHR-6390, XZP-3287, BPI-1178, PF-06873600, NUV-422, FCN-437, seliciclib, mevociclib, milciclib, fadraciclib, zotiraciclib, dinaciclib, samuraciclib, voruciclib, FIT-039, PF-07104091, BEY-1107, panitumumab, sutetinib, allitinib, epitinib, xiliertinib, rociletinib, dacomitinib, simotinib, olmutinib, yinlitinib, mefatinib, alflutinib, almonertinib, icotinib, naquotinib, poziotinib, epertinib, sapitinib, cipatinib, tarloxotinib, pyrotinib, pirotinib, lazertinib, varlitinib, tesevatinib, canertinib, mobocertinib, duligotuzumab, olafertinib, zorifertinib, pelitinib, DZD-9008, ASK-120067, BPI-7711, QLNC-120, ametumumab, imgatuzumab, amivantamab, seribantumab, nimotuzumab, serclutamab, depatuxizumab, tomuzotuximab, SCT-200, ravoxertinib, LY3214996, MK-8353, LTT462, HH-2710, infigratinib, pemigatinib, orantinib, derazantinib, roblitinib, rogaratinib, zoligratinib, E-7090, AZD-4547, ODM-203, ICP-192, HMPL-453, bemarituzumab, quizartinib, crenolanib, flysyn, mivavotinib, PHI-101, MEN-1703, FF-10101, HM-43239, E-6201, ENMD-2076, larotinib, tucatinib (irbinitinib), BDTX-189, trastuzumab, pertuzumab, zanidatamab, zenocutuzumab, margetuximab, KN-026, BAT-8001, TAA-013, KL-A166, selumetinib, refametinib, mirdametinib, pimasertib, HL-085, NFX-179, nilotinib, dovitinib, axitinib, vatalinib, pazopanib, avapritinib, famitinib, catequentinib, necuparanib, surufatinib, lucitanib, midostaurin, vorolanib, bevasiranib, bevacizumab, ranibizumab, vanucizumab, navicixizumab, ramucirumab, BAT-5906, VGX-100, CSL-346, duvelisib, copanlisib, buparlisib, paxalisib, voxtalisib, zandelisib, dezapelisib, linperlisib, inavolisib, parsaclisib, eganelisib, nemiralisib, seletalisib, gedatolisib, leniolisib, tenalisib, pictilisib, bimiralisib, BBP-681, BGB-10188, MEN-1611, ASN-003, ACP-319, panobinostat, resminostat, abexinostat, romidepsin, belinostat, entinostat, quisinostat, pracinostat, tefinostat, mocetinostat, givinostat, dacinostat, ivaltinostat, domatinostat, fimepinostat, tinostamustine, Remetinostat, tucidinostat, ricolinostat, CXD-101, REC-2282, veltuzumab, rituximab, ublituximab, nogitecan, simmitecan, gimatecan, topotecan, cositecan, belotecan, govitecan, deruxtecan, AR-67, camsirubicin, Aldoxorubicin, vosaroxin, mitoxantrone, evofosfamide, amrubicin, sobuzoxane, epirubicin, F-14512, dacarbazine, temozolomide, nimustine, busulfan, procarbazine, melphalan, mitomycin C, daunorubicin, vincristine, vinblastine, vinorelbine, eribulin, trifluridine, docetaxel, cabazitaxel, avanbulin, fluorapacin, mertansine, carboplatin, nedaplatin, ixazomib, marizomib, carfilzomib and LXE-408.

### Item 16

The antitumor effect enhancer according to any one of items 9 to 15, wherein the malignant tumor is selected from the group consisting of epithelial cancer (respiratory cancer, gastrointestinal cancer, genital cancer, cancer of the secretory system, breast cancer, etc.), mesothelioma, sarcoma, hematopoietic tumor, tumors of the central nervous system, retinoblastoma, and tumors of the peripheral nervous system.

### Item 17

An antitumor agent comprising a compound represented by formula (I)

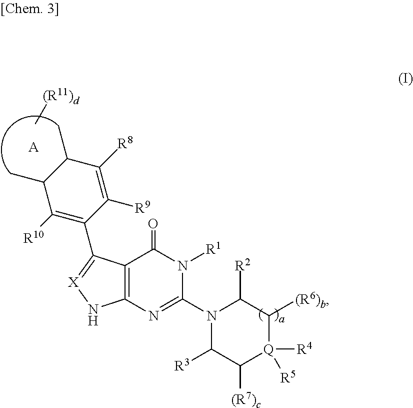

or a tautomer thereof, a solvate thereof, or a pharmaceutically acceptable salt thereof, wherein:

### X is CH or N;

R¹ is —CH₃;
R² and R³ are independently selected from the group consisting of hydrogen and C₁₋₄alkyl;

### Q is C or N;

wherein when Q is C, then either:
   (i) R⁴ is amino, aminoC₁₋₄alkyl, or monoC₁₋₄alkylamino; R⁵ is hydrogen, C₁₋₄alkyl, halogen, hydroxyC₁₋₄alkyl, C₁₋₄alkoxy, haloC₁₋₄alkyl, or C₁₋₄alkoxyC₁₋₄alkyl;
   or
   (ii) R⁴ and R⁵ together with Q form a four- to six-membered ring that can optionally contain 1 to 3 heteroatoms or groups independently selected from the group consisting of N, O, S, NH, C(O), and S(O)ₘ, and said ring formed by R⁴ and R³ can be unsubstituted or substituted with 1 to 4 groups independently selected from the group consisting of amino, halogen, haloC₁₋₄alkyl, hydroxyl, methoxy, methylamino, and C₁₋₄alkyl, and m is selected from the group consisting of 1 and 2; and
wherein when Q is N then:
   R⁴ is absent; and
   R⁵ is hydrogen;
      R⁶ and R⁷ are independently selected from the group consisting of halogen, C₁₋₄alkyl, hydroxyC₁₋₄alkyl, and hydroxyl, provided that when Q is N, then R⁶ or R⁷ is not halogen or hydroxyl;
      or any two groups selected from the group consisting of R², R³, R⁶, and R⁷ together form a one- to three-membered bridge group selected from the group consisting of C₁₋₃alkylene, C₂₋₃alkenylene, methylene-NR^{q}-methylene, and methylene-O-methylene, wherein the bridge group is optionally substituted with a group selected from the group consisting of C₁₋₄alkyl, hydroxyl, and halogen, and R^{q} is selected from the group consisting of hydrogen and C₁₋₄alkyl;
      or R⁴ and R⁷ form a four- to six-membered ring containing a N atom;
      or R³ and R⁷ form a three- to six-membered ring;
      or R⁶ and R⁷ form a direct bond;
      a is selected from the group consisting of 0, 1, and 2;
      b is selected from the group consisting of 0, 1, and 2;
      c is selected from the group consisting of 0, 1, and 2;
      or Q is C, c is 2, R⁴ is hydrogen, and the two R⁷ join to form a 4 to 6-membered nitrogen containing ring;
      Ring A is either:
(i) a five-membered nitrogen-containing heterocyclic ring wherein the heterocyclic ring optionally contains one or two additional heteroatoms selected from the group consisting of N, O, and S, or
(ii) a six-membered aromatic nitrogen-containing heterocyclic ring, wherein the heterocyclic ring optionally contains one or two additional heteroatoms selected from the group consisting of N, O, and S; or
(iii) a six-membered non-aromatic nitrogen-containing heterocyclic ring, wherein the heterocyclic ring optionally contains one or two additional heteroatoms selected from the group consisting of N and S;
   R⁸ is selected from the group consisting of hydrogen, C₁₋₄alkyl, haloC₁₋₄alkyl, and halogen;
   R⁹ is selected from the group consisting of hydrogen and halogen;
   R¹⁰ is selected from the group consisting of haloC₁₋₄alkyl, C₁₋₄alkyl, halogen, hydrogen, and C₁₋₄alkoxy;
   R¹¹ are independently selected from the group consisting of halogen, cyano, cyanoC₁₋₄alkyl, hydroxyl, oxo (═O), C₁₋₄alkyl optionally substituted with a five- or six-membered heterocyclic group containing 1 or 2 heteroatoms selected from the group consisting of O, N, and S, haloC₁₋₄alkyl, C₁₋₄alkoxy, hydroxylC₁₋₄alkyl, C₁₋₄alkoxyC₁₋₄alkyl, C₁₋₄alkylsulfone, amino, monoC₁₋₄alkylamino, diC₁₋₄alkylamino, aminoC₁₋₄alkyl, —C₁₋₄alkylene-C(═O)NH_{(2-q)}(C₁₋₆alkyl)_{q}), —C₁₋₄alkylene-NHC(═O)C₁₋₆alkyl, sulfonamide, sulfonamideC₁₋₄alkyl, 3 to 6-membered cycloalkyl, C₁₋₄alkyl substituted with 3 to 6-membered cycloalkyl, a five- or six-membered unsaturated heterocyclic group containing 1, 2, 3 or 4 heteroatoms selected from the group consisting of 0, N, and S, and an optionally substituted four- to six-membered saturated heterocyclic group containing 1 or 2 heteroatoms selected from the group consisting of O, N, and S where the optional substituent is selected from C₁₋₄alkyl;
   q is selected from the group consisting of 0, 1, and 2; and
   d is selected from the group consisting of 0, 1, and 2,
   wherein the antitumor agent is concomitantly used with at least one additional compound having an antitumor effect, at least one tautomer thereof, at least one solvate thereof, or at least one pharmaceutically acceptable salt thereof, selected from the group consisting of molecular targeted drugs and cytotoxic drugs.

### Item 18

The antitumor agent according to item 17, wherein the compound represented by formula (I), the tautomer thereof, the solvate thereof, or the pharmaceutically acceptable salt thereof is administered before, simultaneously with, or after administration of the at least one additional compound having an antitumor effect, the at least one tautomer thereof, the at least one solvate thereof, or the at least one pharmaceutically acceptable salt thereof.

### Item 19

The antitumor agent according to item 17 or 18, wherein the compound represented by formula (I) is selected from the group consisting of 2-((1R,2R,4S)-2-amino-7-azabicyclo[2.2.1]heptan-7-yl)-5-(3,4-dichloro-2-methyl-2H-indazol-5-yl)-3-methyl-3,7-dihydro-4H-pyrrolo[2,3-d]pyrimidin-4-one, 6-((1R,4R,7R)-7-amino-2-azabicyclo[2.2.1]heptan-2-yl)-3-(3,4-dichloro-2-methyl-2H-indazol-5-yl)-5-methyl-1,5-dihydro-4H-pyrazolo[3,4-d]pyrimidin-4-one, 2-((1R,4R,7R)-7-amino-2-azabicyclo[2.2.1]heptan-2-yl)-5-(4-chloro-2-ethyl-2H-indazol-5-yl)-3-methyl-3,7-dihydro-4H-pyrrolo[2,3-d]pyrimidin-4-one, 2-((1R,4R,7R)-7-amino-2-azabicyclo[2.2.1]heptan-2-yl)-5-(4-chloro-2-methyl-2H-indazol-5-yl)-3-methyl-3,7-dihydro-4H-pyrrolo[2,3-d]pyrimidin-4-one, 2-((1R,4R,7R)-7-amino-2-azabicyclo[2.2.1]heptan-2-yl)-5-(3,4-dichloro-2-methyl-2H-indazol-5-yl)-3-methyl-3,7-dihydro-4H-pyrrolo[2,3-d]pyrimidin-4-one, 2-((1R,2R,4S)-2-amino-7-azabicyclo[2.2.1]heptan-7-yl)-5-(3-chloro-4-fluoro-2-methyl-2H-indazol-5-yl)-3-methyl-3,7-dihydro-4H-pyrrolo[2,3-d]pyrimidin-4-one, and 2-((1R,2R,4S)-2-amino-7-azabicyclo[2.2.1]heptan-7-yl)-5-(4-chloro-2-ethyl-2H-indazol-5-yl)-3-methyl-3,7-dihydro-4H-pyrrolo[2,3-d]pyrimidin-4-one.

### Item 20

The antitumor agent according to any one of items 17 to 19, wherein the compound represented by formula (I) is 2-((1R,2R,4S)-2-amino-7-azabicyclo[2.2.1]heptan-7-yl)-5-(3,4-dichloro-2-methyl-2H-indazol-5-yl)-3-methyl-3,7-dihydro-4H-pyrrolo[2,3-d]pyrimidin-4-one.

### Item 21

The antitumor agent according to any one of items 17 to 20, wherein the at least one additional compound having an antitumor effect, the at least one tautomer thereof, the at least one solvate thereof, or the at least one pharmaceutically acceptable salt thereof is selected from the group consisting of a tyrosine kinase inhibitor, a RAS-MAPK pathway inhibitor, a PI3K pathway inhibitor, a BCL2 inhibitor, a CDK4/6 inhibitor, an HDAC inhibitor, a topoisomerase inhibitor (a topoisomerase I inhibitor, and topoisomerase II inhibitor), an alkylating agent, an anthracycline antibiotic, an alkaloid, an anti-metabolite, an anti-microtubule agent, a platinum-containing drug, a proteasome inhibitor, and a thalidomide analog drug.

### Item 22

The antitumor agent according to any one of items 17 to 21, wherein the molecular targeted drug is selected from the group consisting of an AKT inhibitor, ALK inhibitor, Bcl2 inhibitor, BCR-ABL inhibitor, BRAF inhibitor, CDK4/6 inhibitor, CDK inhibitor, EGFR inhibitor, Erk1/2 inhibitor, FGFR inhibitor, FLT3 inhibitor, HER2 inhibitor, MEK inhibitor, Multi-kinase inhibitor, PI3K inhibitor, RAF inhibitor, HDAC inhibitor, topoisomerase I inhibitor, topoisomerase II inhibitor, alkylating agent, anthracycline antibiotics, alkaloid, anti-metabolite, anti-microtubule agent, platinum-containing drug, and proteasome inhibitor.

### Item 23

The antitumor agent according to any one of items 17 to 22, wherein the molecular targeted drug is selected from the group consisting of cetuximab, MK-2206, alectinib, crizotinib, venetoclax, imatinib, dasatinib, ponatinib, dabrafenib, vemurafenib, sorafenib, palbociclib, abemaciclib, osimertinib, gefitinib, erlotinib, afatinib, brigatinib, ulixertinib, (2R)-2-(6-{5-chloro-2-[(oxan-4-yl)amino]pyrimidin-4-yl}-1-oxo-2,3-dihydro-1H-isoindol-2-yl)-N-[(1S)-1-(3-fluoro-5-methoxyphenyl)-2-hydroxyethyl]propanamide or a salt thereof, erdafitinib, giltertinib, lapatinib, neratinib, trametinib, cobimetinib, binimetinib, regorafenib, sunitinib, nintedanib, anlotinib, vandetanib, lenvatinib, alpelisib and idelalisib/CAL-101, vorinostat (SAHA), irinotecan (SN-38), etoposide, cyclophosphamide, doxorubicin, gemcitabine, pemetrexed, 5-FU, FdUrd, FTD, paclitaxel, cisplatin, oxaliplatin, bortezomib, afuresertib, trans-3-amino-1-methyl-3-(4-(3-phenyl-5H-imidazo[1,2-c]pyrido[3,4-e][1,3]oxazin-2-yl)phenyl)cyclobutanol, capivasertib, ipatasertib, triciribine, miransertib, lorlatinib, ceritinib, repotrectinib, ensartinib, alkotinib, WX-0593, SAF-189s, CT-707, TQ-B3101, sabutoclax, apogossypol, obatoclax, navitoclax, APG-2575, APG-1252, asciminib, olverembatinib, encorafenib, lifirafenib, LXH-254, ribociclib, lerociclib, trilaciclib, alvocidib, GLR-2007, SHR-6390, XZP-3287, BPI-1178, PF-06873600, NUV-422, FCN-437, seliciclib, mevociclib, milciclib, fadraciclib, zotiraciclib, dinaciclib, samuraciclib, voruciclib, FIT-039, PF-07104091, BEY-1107, panitumumab, sutetinib, allitinib, epitinib, xiliertinib, rociletinib, dacomitinib, simotinib, olmutinib, yinlitinib, mefatinib, alflutinib, almonertinib, icotinib, naquotinib, poziotinib, epertinib, sapitinib, cipatinib, tarloxotinib, pyrotinib, pirotinib, lazertinib, varlitinib, tesevatinib, canertinib, mobocertinib, duligotuzumab, olafertinib, zorifertinib, pelitinib, DZD-9008, ASK-120067, BPI-7711, QLNC-120, ametumumab, imgatuzumab, amivantamab, seribantumab, nimotuzumab, serclutamab, depatuxizumab, tomuzotuximab, SCT-200, ravoxertinib, LY3214996, MK-8353, LTT462, HH-2710, infigratinib, pemigatinib, orantinib, derazantinib, roblitinib, rogaratinib, zoligratinib, E-7090, AZD-4547, ODM-203, ICP-192, HMPL-453, bemarituzumab, quizartinib, crenolanib, flysyn, mivavotinib, PHI-101, MEN-1703, FF-10101, HM-43239, E-6201, ENMD-2076, larotinib, tucatinib (irbinitinib), BDTX-189, trastuzumab, pertuzumab, zanidatamab, zenocutuzumab, margetuximab, KN-026, BAT-8001, TAA-013, KL-A166, selumetinib, refametinib, mirdametinib, pimasertib, HL-085, NFX-179, nilotinib, dovitinib, axitinib, vatalinib, pazopanib, avapritinib, famitinib, catequentinib, necuparanib, surufatinib, lucitanib, midostaurin, vorolanib, bevasiranib, bevacizumab, ranibizumab, vanucizumab, navicixizumab, ramucirumab, BAT-5906, VGX-100, CSL-346, duvelisib, copanlisib, buparlisib, paxalisib, voxtalisib, zandelisib, dezapelisib, linperlisib, inavolisib, parsaclisib, eganelisib, nemiralisib, seletalisib, gedatolisib, leniolisib, tenalisib, pictilisib, bimiralisib, BBP-681, BGB-10188, MEN-1611, ASN-003, ACP-319, panobinostat, resminostat, abexinostat, romidepsin, belinostat, entinostat, quisinostat, pracinostat, tefinostat, mocetinostat, givinostat, dacinostat, ivaltinostat, domatinostat, fimepinostat, tinostamustine, Remetinostat, tucidinostat, ricolinostat, CXD-101, REC-2282, veltuzumab, rituximab, ublituximab, nogitecan, simmitecan, gimatecan, topotecan, cositecan, belotecan, govitecan, deruxtecan, AR-67, camsirubicin, Aldoxorubicin, vosaroxin, mitoxantrone, evofosfamide, amrubicin, sobuzoxane, epirubicin, F-14512, dacarbazine, temozolomide, nimustine, busulfan, procarbazine, melphalan, mitomycin C, daunorubicin, vincristine, vinblastine, vinorelbine, eribulin, trifluridine, docetaxel, cabazitaxel, avanbulin, fluorapacin, mertansine, carboplatin, nedaplatin, ixazomib, marizomib, carfilzomib and LXE-408.

### Item 24

The antitumor agent enhancer according to any one of items 17 to 23, wherein the malignant tumor is selected from the group consisting of epithelial cancer (respiratory cancer, gastrointestinal cancer, genital cancer, cancer of the secretory system, breast cancer, etc.), mesothelioma, sarcoma, hematopoietic tumor, tumors of the central nervous system, retinoblastoma, and tumors of the peripheral nervous system.

### Item 25

Use of a compound represented by formula (I)

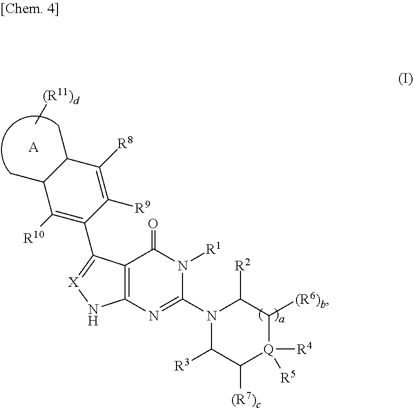

or a tautomer thereof, a solvate thereof, or a pharmaceutically acceptable salt thereof, wherein:

### X is CH or N;

R¹ is —CH₃;
R² and R³ are independently selected from the group consisting of hydrogen and C₁₋₄alkyl;

### Q is C or N;

wherein when Q is C, then either:
   (i) R⁴ is amino, aminoC₁₋₄alkyl, or monoC₁₋₄alkylamino; R⁵ is hydrogen, C₁₋₄alkyl, halogen, hydroxyC₁₋₄alkyl, C₁₋₄alkoxy, haloC₁₋₄alkyl, or C₁₋₄alkoxyC₁₋₄alkyl;
   or
   (ii) R⁴ and R⁵ together with Q form a four- to six-membered ring that can optionally contain 1 to 3 heteroatoms or groups independently selected from the group consisting of N, O, S, NH, C(O), and S(O)ₘ, and said ring formed by R⁴ and R³ can be unsubstituted or substituted with 1 to 4 groups independently selected from the group consisting of amino, halogen, haloC₁₋₄alkyl, hydroxyl, methoxy, methylamino, and C₁₋₄alkyl, and m is selected from the group consisting of 1 and 2; and
wherein when Q is N, then:
   R⁴ is absent; and
   R⁵ is hydrogen;
      R⁶ and R⁷ are independently selected from the group consisting of halogen, C₁₋₄alkyl, hydroxyC₁₋₄alkyl, and hydroxyl, provided that when Q is N, then R⁶ or R⁷ is not halogen or hydroxyl;
      or any two groups selected from the group consisting of R², R³, R⁶, and R⁷ together form a one- to three-membered bridge group selected from the group consisting of C₁₋₃alkylene, C₂₋₃alkenylene, methylene-NR^{q}-methylene, and methylene-O-methylene, wherein the bridge group is optionally substituted with a group selected from the group consisting of C₁₋₄alkyl, hydroxyl, and halogen, and R^{q} is selected from the group consisting of hydrogen and C₁₋₄alkyl;
      or R⁴ and R⁷ form a four- to six-membered ring containing a N atom;
      or R⁵ and R⁷ form a three- to six-membered ring;
      or R⁶ and R⁷ form a direct bond;
      a is selected from the group consisting of 0, 1, and 2;
      b is selected from the group consisting of 0, 1, and 2;
      c is selected from the group consisting of 0, 1, and 2;
      or Q is C, c is 2, R⁴ is hydrogen, and the two R⁷ join to form a 4 to 6-membered nitrogen containing ring;
      Ring A is either:
(i) a five-membered nitrogen-containing heterocyclic ring wherein the heterocyclic ring optionally contains one or two additional heteroatoms selected from the group consisting of N, O, and S, or
(ii) a six-membered aromatic nitrogen-containing heterocyclic ring, wherein the heterocyclic ring optionally contains one or two additional heteroatoms selected from the group consisting of N, O, and S; or
(iii) a six-membered non-aromatic nitrogen-containing heterocyclic ring, wherein the heterocyclic ring optionally contains one or two additional heteroatoms selected from the group consisting of N and S;
   R⁸ is selected from the group consisting of hydrogen, C₁₋₄alkyl, haloC₁₋₄alkyl, and halogen;
   R⁹ is selected from the group consisting of hydrogen and halogen;
   R¹⁰ is selected from the group consisting of haloC₁₋₄alkyl, C₁₋₄alkyl, halogen, hydrogen, and C₁₋₄alkoxy;
   R¹¹ are independently selected from the group consisting of halogen, cyano, cyanoC₁₋₄alkyl, hydroxyl, oxo (═O), C₁₋₄alkyl optionally substituted with a five- or six-membered heterocyclic group containing 1 or 2 heteroatoms selected from the group consisting of O, N, and S, haloC₁₋₄alkyl, C₁₋₄alkoxy, hydroxylC₁₋₄alkyl, C₁₋₄alkoxyC₁₋₄alkyl, C₁₋₄alkylsulfone, amino, monoC₁₋₄alkylamino, diC₁₋₄alkylamino, aminoC₁₋₄alkyl, —C₁₋₄alkylene-C(═O)NH_{(2-q)}(C₁₋₆alkyl)_{q}), —C₁₋₄alkylene-NHC(═O)C₁₋₆alkyl, sulfonamide, sulfonamideC₁₋₄alkyl, 3 to 6-membered cycloalkyl, C₁₋₄alkyl substituted with 3 to 6-membered cycloalkyl, a five- or six-membered unsaturated heterocyclic group containing 1, 2, 3 or 4 heteroatoms selected from the group consisting of 0, N, and S, and an optionally substituted four- to six-membered saturated heterocyclic group containing 1 or 2 heteroatoms selected from the group consisting of O, N, and S where the optional substituent is selected from C₁₋₄alkyl;
   q is selected from the group consisting of 0, 1, and 2; and
   d is selected from the group consisting of 0, 1, and 2,
   in the enhancement of the antitumor effect of at least one additional compound having an antitumor effect, at least one tautomer thereof, at least one solvate thereof, or at least one pharmaceutically acceptable salt thereof, selected from the group consisting of molecular targeted drugs and cytotoxic drugs.

### Item 26

The use according to item 25, wherein the compound represented by formula (I), the tautomer thereof, the solvate thereof, or the pharmaceutically acceptable salt thereof is administered before, simultaneously with, or after administration of the at least one additional compound having an antitumor effect, the at least one tautomer thereof, the at least one solvate thereof, or the at least one pharmaceutically acceptable salt thereof.

### Item 27

The use according to item 25 or 26, wherein the compound represented by formula (I) is selected from the group consisting of 2-((1R,2R,4S)-2-amino-7-azabicyclo[2.2.1]heptan-7-yl)-5-(3,4-dichloro-2-methyl-2H-indazol-5-yl)-3-methyl-3,7-dihydro-4H-pyrrolo[2,3-d]pyrimidin-4-one, 6-((1R,4R,7R)-7-amino-2-azabicyclo[2.2.1]heptan-2-yl)-3-(3,4-dichloro-2-methyl-2H-indazol-5-yl)-5-methyl-1,5-dihydro-4H-pyrazolo[3,4-d]pyrimidin-4-one, 2-((1R,4R,7R)-7-amino-2-azabicyclo[2.2.1]heptan-2-yl)-5-(4-chloro-2-ethyl-2H-indazol-5-yl)-3-methyl-3,7-dihydro-4H-pyrrolo[2,3-d]pyrimidin-4-one, 2-((1R,4R,7R)-7-amino-2-azabicyclo[2.2.1]heptan-2-yl)-5-(4-chloro-2-methyl-2H-indazol-5-yl)-3-methyl-3,7-dihydro-4H-pyrrolo[2,3-d]pyrimidin-4-one, 2-((1R,4R,7R)-7-amino-2-azabicyclo[2.2.1]heptan-2-yl)-5-(3,4-dichloro-2-methyl-2H-indazol-5-yl)-3-methyl-3,7-dihydro-4H-pyrrolo[2,3-d]pyrimidin-4-one, 2-((1R,2R,4S)-2-amino-7-azabicyclo[2.2.1]heptan-7-yl)-5-(3-chloro-4-fluoro-2-methyl-2H-indazol-5-yl)-3-methyl-3,7-dihydro-4H-pyrrolo[2,3-d]pyrimidin-4-one, and 2-((1R,2R,4S)-2-amino-7-azabicyclo[2.2.1]heptan-7-yl)-5-(4-chloro-2-ethyl-2H-indazol-5-yl)-3-methyl-3,7-dihydro-4H-pyrrolo[2,3-d]pyrimidin-4-one.

### Item 28

The use according to any one of items 25 to 27, wherein the compound represented by formula (I) is 2-((1R,2R,4S)-2-amino-7-azabicyclo[2.2.1]heptan-7-yl)-5-(3,4-dichloro-2-methyl-2H-indazol-5-yl)-3-methyl-3,7-dihydro-4H-pyrrolo[2,3-d]pyrimidin-4-one.

### Item 29

The use according to any one of items 25 to 28, wherein the at least one additional compound having an antitumor effect, the at least one tautomer thereof, the at least one solvate thereof, or the at least one pharmaceutically acceptable salt thereof is selected from the group consisting of a tyrosine kinase inhibitor, a RAS-MAPK pathway inhibitor, a PI3K pathway inhibitor, a BCL2 inhibitor, a CDK4/6 inhibitor, an HDAC inhibitor, a topoisomerase inhibitor (a topoisomerase I inhibitor, and topoisomerase II inhibitor), an alkylating agent, an anthracycline antibiotic, an alkaloid, an anti-metabolite, anti-microtubule agent, a platinum-containing drug, a proteasome inhibitor, and a thalidomide analog drug.

### Item 30

The use according to any one of items 25 to 29, wherein the molecular targeted drug is selected from the group consisting of an AKT inhibitor, ALK inhibitor, Bcl2 inhibitor, BCR-ABL inhibitor, BRAF inhibitor, CDK4/6 inhibitor, CDK inhibitor, EGFR inhibitor, Erk1/2 inhibitor, FGFR inhibitor, FLT3 inhibitor, HER2 inhibitor, MEK inhibitor, Multi-kinase inhibitor, PI3K inhibitor, RAF inhibitor, HDAC inhibitor, topoisomerase I inhibitor, topoisomerase II inhibitor, alkylating agent, anthracycline antibiotics, alkaloid, anti-metabolite, anti-microtubule agent, platinum-containing drug, and proteasome inhibitor.

### Item 31

The use according to any one of items 25 to 30, wherein the molecular targeted drug is selected from the group consisting of cetuximab, MK-2206, alectinib, crizotinib, venetoclax, imatinib, dasatinib, ponatinib, dabrafenib, vemurafenib, sorafenib, palbociclib, abemaciclib, osimertinib, gefitinib, erlotinib, afatinib, brigatinib, ulixertinib, (2R)-2-(6-{5-chloro-2-[(oxan-4-yl)amino]pyrimidin-4-yl}-1-oxo-2,3-dihydro-1H-isoindol-2-yl)-N-[(1S)-1-(3-fluoro-5-methoxyphenyl)-2-hydroxyethyl]propanamide or a salt thereof, erdafitinib, giltertinib, lapatinib, neratinib, trametinib, cobimetinib, binimetinib, regorafenib, sunitinib, nintedanib, anlotinib, vandetanib, lenvatinib, alpelisib and idelalisib/CAL-101, vorinostat (SAHA), irinotecan (SN-38), etoposide, cyclophosphamide, doxorubicin, gemcitabine, pemetrexed, 5-FU, FdUrd, FTD, paclitaxel, cisplatin, oxaliplatin, bortezomib, afuresertib, trans-3-amino-1-methyl-3-(4-(3-phenyl-5H-imidazo[1,2-c]pyrido[3,4-e][1,3]oxazin-2-yl)phenyl)cyclobutanol, capivasertib, ipatasertib, triciribine, miransertib, lorlatinib, ceritinib, repotrectinib, ensartinib, alkotinib, WX-0593, SAF-189s, CT-707, TQ-B3101, sabutoclax, apogossypol, obatoclax, navitoclax, APG-2575, APG-1252, asciminib, olverembatinib, encorafenib, lifirafenib, LXH-254, ribociclib, lerociclib, trilaciclib, alvocidib, GLR-2007, SHR-6390, XZP-3287, BPI-1178, PF-06873600, NUV-422, FCN-437, seliciclib, mevociclib, milciclib, fadraciclib, zotiraciclib, dinaciclib, samuraciclib, voruciclib, FIT-039, PF-07104091, BEY-1107, panitumumab, sutetinib, allitinib, epitinib, xiliertinib, rociletinib, dacomitinib, simotinib, olmutinib, yinlitinib, mefatinib, alflutinib, almonertinib, icotinib, naquotinib, poziotinib, epertinib, sapitinib, cipatinib, tarloxotinib, pyrotinib, pirotinib, lazertinib, varlitinib, tesevatinib, canertinib, mobocertinib, duligotuzumab, olafertinib, zorifertinib, pelitinib, DZD-9008, ASK-120067, BPI-7711, QLNC-120, ametumumab, imgatuzumab, amivantamab, seribantumab, nimotuzumab, serclutamab, depatuxizumab, tomuzotuximab, SCT-200, ravoxertinib, LY3214996, MK-8353, LTT462, HH-2710, infigratinib, pemigatinib, orantinib, derazantinib, roblitinib, rogaratinib, zoligratinib, E-7090, AZD-4547, ODM-203, ICP-192, HMPL-453, bemarituzumab, quizartinib, crenolanib, flysyn, mivavotinib, PHI-101, MEN-1703, FF-10101, HM-43239, E-6201, ENMD-2076, larotinib, tucatinib (irbinitinib), BDTX-189, trastuzumab, pertuzumab, zanidatamab, zenocutuzumab, margetuximab, KN-026, BAT-8001, TAA-013, KL-A166, selumetinib, refametinib, mirdametinib, pimasertib, HL-085, NFX-179, nilotinib, dovitinib, axitinib, vatalinib, pazopanib, avapritinib, famitinib, catequentinib, necuparanib, surufatinib, lucitanib, midostaurin, vorolanib, bevasiranib, bevacizumab, ranibizumab, vanucizumab, navicixizumab, ramucirumab, BAT-5906, VGX-100, CSL-346, duvelisib, copanlisib, buparlisib, paxalisib, voxtalisib, zandelisib, dezapelisib, linperlisib, inavolisib, parsaclisib, eganelisib, nemiralisib, seletalisib, gedatolisib, leniolisib, tenalisib, pictilisib, bimiralisib, BBP-681, BGB-10188, MEN-1611, ASN-003, ACP-319, panobinostat, resminostat, abexinostat, romidepsin, belinostat, entinostat, quisinostat, pracinostat, tefinostat, mocetinostat, givinostat, dacinostat, ivaltinostat, domatinostat, fimepinostat, tinostamustine, Remetinostat, tucidinostat, ricolinostat, CXD-101, REC-2282, veltuzumab, rituximab, ublituximab, nogitecan, simmitecan, gimatecan, topotecan, cositecan, belotecan, govitecan, deruxtecan, AR-67, camsirubicin, Aldoxorubicin, vosaroxin, mitoxantrone, evofosfamide, amrubicin, sobuzoxane, epirubicin, F-14512, dacarbazine, temozolomide, nimustine, busulfan, procarbazine, melphalan, mitomycin C, daunorubicin, vincristine, vinblastine, vinorelbine, eribulin, trifluridine, docetaxel, cabazitaxel, avanbulin, fluorapacin, mertansine, carboplatin, nedaplatin, ixazomib, marizomib, carfilzomib and LXE-408.

### Item 32

The use according to any one of items 25 to 31, wherein the malignant tumor is selected from the group consisting of epithelial cancer (respiratory cancer, gastrointestinal cancer, genital cancer, cancer of the secretory system, breast cancer, etc.), mesothelioma, sarcoma, hematopoietic tumor, tumors of the central nervous system, retinoblastoma, and tumors of the peripheral nervous system.

### Item 33

A method for treating a tumor comprising administering a compound represented by formula (I)

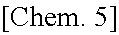

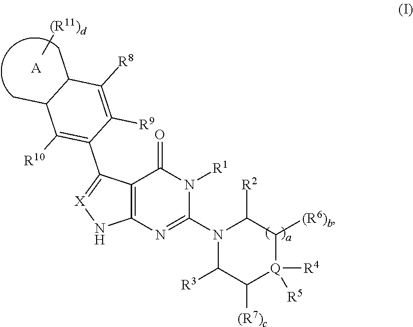

or a tautomer thereof, a solvate thereof, or a pharmaceutically acceptable salt thereof, wherein:

### X is CH or N;

R¹ is —CH₃;
R² and R³ are independently selected from the group consisting of hydrogen and C₁₋₄alkyl;

### Q is C or N;

wherein when Q is C, then either:
   (i) R⁴ is amino, aminoC₁₋₄alkyl, or monoC₁₋₄alkylamino; R⁵ is hydrogen, C₁₋₄alkyl, halogen, hydroxyC₁₋₄alkyl, C₁₋₄alkoxy, haloC₁₋₄alkyl, or C₁₋₄alkoxyC₁₋₄alkyl;
   or
   (ii) R⁴ and R⁵ together with Q form a four- to six-membered ring that can optionally contain 1 to 3 heteroatoms or groups independently selected from the group consisting of N, O, S, NH, C(O), and S(O)ₘ, and said ring formed by R⁴ and R³ can be unsubstituted or substituted with 1 to 4 groups independently selected from the group consisting of amino, halogen, haloC₁₋₄alkyl, hydroxyl, methoxy, methylamino, and C₁₋₄alkyl, and m is selected from the group consisting of 1 and 2; and
wherein when Q is N, then:
   R⁴ is absent; and
   R⁵ is hydrogen;
      R⁶ and R⁷ are independently selected from the group consisting of halogen, C₁₋₄alkyl, hydroxyC₁₋₄alkyl, and hydroxyl, provided that when Q is N, then R⁶ or R⁷ is not halogen or hydroxyl;
      or any two groups selected from the group consisting of R², R³, R⁶, and R⁷ together form a one- to three-membered bridge group selected from the group consisting of C₁₋₃alkylene, C₂₋₃alkenylene, methylene-NR^{q}-methylene, and methylene-O-methylene, wherein the bridge group is optionally substituted with a group selected from the group consisting of C₁₋₄alkyl, hydroxyl, and halogen, and R^{q} is selected from the group consisting of hydrogen and C₁₋₄alkyl;
      or R⁴ and R⁷ form a four- to six-membered ring containing a N atom;
      or R³ and R⁷ form a three- to six-membered ring;
      or R⁶ and R⁷ form a direct bond;
      a is selected from the group consisting of 0, 1, and 2;
      b is selected from the group consisting of 0, 1, and 2;
      c is selected from the group consisting of 0, 1, and 2;
      or Q is C, c is 2, R⁴ is hydrogen, and the two R⁷ join to form a 4 to 6-membered nitrogen containing ring;
      Ring A is either:
(i) a five-membered nitrogen-containing heterocyclic ring wherein the heterocyclic ring optionally contains one or two additional heteroatoms selected from the group consisting of N, O, and S, or
(ii) a six-membered aromatic nitrogen-containing heterocyclic ring, wherein the heterocyclic ring optionally contains one or two additional heteroatoms selected from the group consisting of N, O, and S; or
(iii) a six-membered non-aromatic nitrogen-containing heterocyclic ring, wherein the heterocyclic ring optionally contains one or two additional heteroatoms selected from the group consisting of N and S;
   R⁸ is selected from the group consisting of hydrogen, C₁₋₄alkyl, haloC₁₋₄alkyl, and halogen;
   R⁹ is selected from the group consisting of hydrogen and halogen;
   R¹⁰ is selected from the group consisting of haloC₁₋₄alkyl, C₁₋₄alkyl, halogen, hydrogen, and C₁₋₄alkoxy;
   R¹¹ are independently selected from the group consisting of halogen, cyano, cyanoC₁₋₄alkyl, hydroxyl, oxo (═O), C₁₋₄alkyl optionally substituted with a five- or six-membered heterocyclic group containing 1 or 2 heteroatoms selected from the group consisting of O, N, and S, haloC₁₋₄alkyl, C₁₋₄alkoxy, hydroxylC₁₋₄alkyl, C₁₋₄alkoxyC₁₋₄alkyl, C₁₋₄alkylsulfone, amino, monoC₁₋₄alkylamino, diC₁₋₄alkylamino, aminoC₁₋₄alkyl, —C₁₋₄alkylene-C(═O)NH_{(2-q)}(C₁₋₆alkyl)_{q}), —C₁₋₄alkylene-NHC(═O)C₁₋₆alkyl, sulfonamide, sulfonamideC₁₋₄alkyl, 3 to 6-membered cycloalkyl, C₁₋₄alkyl substituted with 3 to 6-membered cycloalkyl, a five- or six-membered unsaturated heterocyclic group containing 1, 2, 3 or 4 heteroatoms selected from the group consisting of 0, N, and S, and an optionally substituted four- to six-membered saturated heterocyclic group containing 1 or 2 heteroatoms selected from the group consisting of O, N, and S where the optional substituent is selected from C₁₋₄alkyl;
   q is selected from the group consisting of 0, 1, and 2; and
   d is selected from the group consisting of 0, 1, and 2,
   and at least one additional compound having an antitumor effect, at least one tautomer thereof, at least one solvate thereof, or at least one pharmaceutically acceptable salt thereof, selected from the group consisting of molecular targeted drugs and cytotoxic drugs.

### Item 34

The method for treating a tumor according to item 33, wherein the compound represented by formula (I), the tautomer thereof, the solvate thereof, or the pharmaceutically acceptable salt thereof is administered before, simultaneously with, or after administration of the at least one additional compound having an antitumor effect, the at least one tautomer thereof, the at least one solvate thereof, or the at least one pharmaceutically acceptable salt thereof.

### Item 35

The method for treating a tumor according to item 33 or 34, wherein the compound represented by formula (I) is selected from the group consisting of 2-((1R,2R,4S)-2-amino-7-azabicyclo[2.2.1]heptan-7-yl)-5-(3,4-dichloro-2-methyl-2H-indazol-5-yl)-3-methyl-3,7-dihydro-4H-pyrrolo[2,3-d]pyrimidin-4-one, 6-((1R,4R,7R)-7-amino-2-azabicyclo[2.2.1]heptan-2-yl)-3-(3,4-dichloro-2-methyl-2H-indazol-5-yl)-5-methyl-1,5-dihydro-4H-pyrazolo[3,4-d]pyrimidin-4-one, 2-((1R,4R,7R)-7-amino-2-azabicyclo[2.2.1]heptan-2-yl)-5-(4-chloro-2-ethyl-2H-indazol-5-yl)-3-methyl-3,7-dihydro-4H-pyrrolo[2,3-d]pyrimidin-4-one, 2-((1R,4R,7R)-7-amino-2-azabicyclo[2.2.1]heptan-2-yl)-5-(4-chloro-2-methyl-2H-indazol-5-yl)-3-methyl-3,7-dihydro-4H-pyrrolo[2,3-d]pyrimidin-4-one, 2-((1R,4R,7R)-7-amino-2-azabicyclo[2.2.1]heptan-2-yl)-5-(3,4-dichloro-2-methyl-2H-indazol-5-yl)-3-methyl-3,7-dihydro-4H-pyrrolo[2,3-d]pyrimidin-4-one, 2-((1R,2R,4S)-2-amino-7-azabicyclo[2.2.1]heptan-7-yl)-5-(3-chloro-4-fluoro-2-methyl-2H-indazol-5-yl)-3-methyl-3,7-dihydro-4H-pyrrolo[2,3-d]pyrimidin-4-one, and 2-((1R,2R,4S)-2-amino-7-azabicyclo[2.2.1]heptan-7-yl)-5-(4-chloro-2-ethyl-2H-indazol-5-yl)-3-methyl-3,7-dihydro-4H-pyrrolo[2,3-d]pyrimidin-4-one.

### Item 36

The method for treating a tumor according to any one of items 33 to 35, wherein the compound represented by formula (I) is 2-((1R,2R,4S)-2-amino-7-azabicyclo[2.2.1]heptan-7-yl)-5-(3,4-dichloro-2-methyl-2H-indazol-5-yl)-3-methyl-3,7-dihydro-4H-pyrrolo[2,3-d]pyrimidin-4-one.

### Item 37

The method for treating a tumor according to any one of items 33 to 36, wherein the at least one additional compound having an antitumor effect, the at least one tautomer thereof, the at least one solvate thereof, or the at least one pharmaceutically acceptable salt thereof is selected from the group consisting of a tyrosine kinase inhibitor, a RAS-MAPK pathway inhibitor, a PI3K pathway inhibitor, a BCL2 inhibitor, a CDK4/6 inhibitor, an HDAC inhibitor, a topoisomerase inhibitor (a topoisomerase I inhibitor, and topoisomerase II inhibitor), an alkylating agent, an anthracycline antibiotic, an alkaloid, an anti-metabolite, an anti-microtubule agent, a platinum-containing drug, a proteasome inhibitor, and a thalidomide analog drug.

### Item 38

The method for treating a tumor according to any one of items 33 to 37, wherein the molecular targeted drug is selected from the group consisting of an AKT inhibitor, ALK inhibitor, Bcl2 inhibitor, BCR-ABL inhibitor, BRAF inhibitor, CDK4/6 inhibitor, CDK inhibitor, EGFR inhibitor, Erk1/2 inhibitor, FGFR inhibitor, FLT3 inhibitor, HER2 inhibitor, MEK inhibitor, Multi-kinase inhibitor, PI3K inhibitor, RAF inhibitor, HDAC inhibitor, topoisomerase I inhibitor, topoisomerase II inhibitor, alkylating agent, anthracycline antibiotics, alkaloid, anti-metabolite, anti-microtubule agent, platinum-containing drug, and proteasome inhibitor.

### Item 39

The method for treating a tumor according to any one of items 33 to 38, wherein the molecular targeted drug is selected from the group consisting of cetuximab, MK-2206, alectinib, crizotinib, venetoclax, imatinib, dasatinib, ponatinib, dabrafenib, vemurafenib, sorafenib, palbociclib, abemaciclib, osimertinib, gefitinib, erlotinib, afatinib, brigatinib, ulixertinib, (2R)-2-(6-{5-chloro-2-[(oxan-4-yl)amino]pyrimidin-4-yl}-1-oxo-2,3-dihydro-1H-isoindol-2-yl)-N-[(1S)-1-(3-fluoro-5-methoxyphenyl)-2-hydroxyethyl]propanamide or a salt thereof, erdafitinib, giltertinib, lapatinib, neratinib, trametinib, cobimetinib, binimetinib, regorafenib, sunitinib, nintedanib, anlotinib, vandetanib, lenvatinib, alpelisib and idelalisib/CAL-101, vorinostat (SAHA), irinotecan (SN-38), etoposide, cyclophosphamide, doxorubicin, gemcitabine, pemetrexed, 5-FU, FdUrd, FTD, paclitaxel, cisplatin, oxaliplatin, bortezomib, afuresertib, trans-3-amino-1-methyl-3-(4-(3-phenyl-5H-imidazo[1,2-c]pyrido[3,4-e][1,3]oxazin-2-yl)phenyl)cyclobutanol, capivasertib, ipatasertib, triciribine, miransertib, lorlatinib, ceritinib, repotrectinib, ensartinib, alkotinib, WX-0593, SAF-189s, CT-707, TQ-B3101, sabutoclax, apogossypol, obatoclax, navitoclax, APG-2575, APG-1252, asciminib, olverembatinib, encorafenib, lifirafenib, LXH-254, ribociclib, lerociclib, trilaciclib, alvocidib, GLR-2007, SHR-6390, XZP-3287, BPI-1178, PF-06873600, NUV-422, FCN-437, seliciclib, mevociclib, milciclib, fadraciclib, zotiraciclib, dinaciclib, samuraciclib, voruciclib, FIT-039, PF-07104091, BEY-1107, panitumumab, sutetinib, allitinib, epitinib, xiliertinib, rociletinib, dacomitinib, simotinib, olmutinib, yinlitinib, mefatinib, alflutinib, almonertinib, icotinib, naquotinib, poziotinib, epertinib, sapitinib, cipatinib, tarloxotinib, pyrotinib, pirotinib, lazertinib, varlitinib, tesevatinib, canertinib, mobocertinib, duligotuzumab, olafertinib, zorifertinib, pelitinib, DZD-9008, ASK-120067, BPI-7711, QLNC-120, ametumumab, imgatuzumab, amivantamab, seribantumab, nimotuzumab, serclutamab, depatuxizumab, tomuzotuximab, SCT-200, ravoxertinib, LY3214996, MK-8353, LTT462, HH-2710, infigratinib, pemigatinib, orantinib, derazantinib, roblitinib, rogaratinib, zoligratinib, E-7090, AZD-4547, ODM-203, ICP-192, HMPL-453, bemarituzumab, quizartinib, crenolanib, flysyn, mivavotinib, PHI-101, MEN-1703, FF-10101, HM-43239, E-6201, ENMD-2076, larotinib, tucatinib (irbinitinib), BDTX-189, trastuzumab, pertuzumab, zanidatamab, zenocutuzumab, margetuximab, KN-026, BAT-8001, TAA-013, KL-A166, selumetinib, refametinib, mirdametinib, pimasertib, HL-085, NFX-179, nilotinib, dovitinib, axitinib, vatalinib, pazopanib, avapritinib, famitinib, catequentinib, necuparanib, surufatinib, lucitanib, midostaurin, vorolanib, bevasiranib, bevacizumab, ranibizumab, vanucizumab, navicixizumab, ramucirumab, BAT-5906, VGX-100, CSL-346, duvelisib, copanlisib, buparlisib, paxalisib, voxtalisib, zandelisib, dezapelisib, linperlisib, inavolisib, parsaclisib, eganelisib, nemiralisib, seletalisib, gedatolisib, leniolisib, tenalisib, pictilisib, bimiralisib, BBP-681, BGB-10188, MEN-1611, ASN-003, ACP-319, panobinostat, resminostat, abexinostat, romidepsin, belinostat, entinostat, quisinostat, pracinostat, tefinostat, mocetinostat, givinostat, dacinostat, ivaltinostat, domatinostat, fimepinostat, tinostamustine, Remetinostat, tucidinostat, ricolinostat, CXD-101, REC-2282, veltuzumab, rituximab, ublituximab, nogitecan, simmitecan, gimatecan, topotecan, cositecan, belotecan, govitecan, deruxtecan, AR-67, camsirubicin, Aldoxorubicin, vosaroxin, mitoxantrone, evofosfamide, amrubicin, sobuzoxane, epirubicin, F-14512, dacarbazine, temozolomide, nimustine, busulfan, procarbazine, melphalan, mitomycin C, daunorubicin, vincristine, vinblastine, vinorelbine, eribulin, trifluridine, docetaxel, cabazitaxel, avanbulin, fluorapacin, mertansine, carboplatin, nedaplatin, ixazomib, marizomib, carfilzomib and LXE-408.

### Item 40

The method for treating a tumor according to any one of items 33 to 39, wherein the malignant tumor is selected from the group consisting of epithelial cancer (respiratory cancer, gastrointestinal cancer, genital cancer, cancer of the secretory system, breast cancer, etc.), mesothelioma, sarcoma, hematopoietic tumor, tumors of the central nervous system, retinoblastoma, and tumors of the peripheral nervous system.

### Item 41

A kit for malignant tumor treatment comprising a compound represented by formula (I)

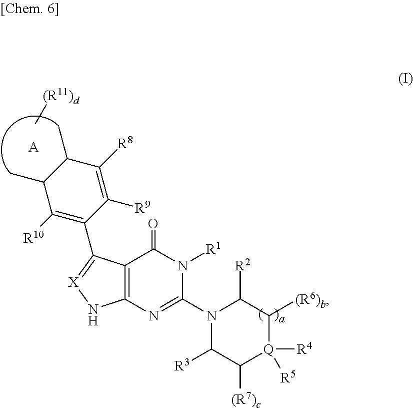

or a tautomer thereof, a solvate thereof, or a pharmaceutically acceptable salt thereof, wherein:

### X is CH or N;

R¹ is —CH₃;
R² and R³ are independently selected from the group consisting of hydrogen and C₁₋₄alkyl;

### Q is C or N;

wherein when Q is C, then either:
   (i) R⁴ is amino, aminoC₁₋₄alkyl, or monoC₁₋₄alkylamino; R⁵ is hydrogen, C₁₋₄alkyl, halogen, hydroxyC₁₋₄alkyl, C₁₋₄alkoxy, haloC₁₋₄alkyl, or C₁₋₄alkoxyC₁₋₄alkyl;
   or
   (ii) R⁴ and R⁵ together with Q form a four- to six-membered ring that can optionally contain 1 to 3 heteroatoms or groups independently selected from the group consisting of N, O, S, NH, C(O), and S(O)ₘ, and said ring formed by R⁴ and R³ can be unsubstituted or substituted with 1 to 4 groups independently selected from the group consisting of amino, halogen, haloC₁₋₄alkyl, hydroxyl, methoxy, methylamino, and C₁₋₄alkyl, and m is selected from the group consisting of 1 and 2; and
wherein when Q is N, then:
   R⁴ is absent; and
   R⁵ is hydrogen;
      R⁶ and R⁷ are independently selected from the group consisting of halogen, C₁₋₄alkyl, hydroxyC₁₋₄alkyl, and hydroxyl provided that when Q is N, then R⁶ or R⁷ is not halogen or hydroxyl;
      or any two groups selected from the group consisting of R², R³, R⁶, and R⁷ together form a one- to three-membered bridge group selected from the group consisting of C₁₋₃alkylene, C₂₋₃alkenylene, methylene-NR^{q}-methylene, and methylene-O-methylene, wherein the bridge group is optionally substituted with a group selected from the group consisting of C₁₋₄alkyl, hydroxyl, and halogen, and R^{q} is selected from the group consisting of hydrogen and C₁₋₄alkyl;
      or R⁴ and R⁷ form a four- to six-membered ring containing a N atom;
      or R³ and R⁷ form a three- to six-membered ring;
      or R⁶ and R⁷ form a direct bond;
      a is selected from the group consisting of 0, 1, and 2;
      b is selected from the group consisting of 0, 1, and 2;
      c is selected from the group consisting of 0, 1, and 2;
      or Q is C, c is 2, R⁴ is hydrogen, and the two R⁷ join to form a 4 to 6-membered nitrogen containing ring;
      Ring A is either:
(i) a five-membered nitrogen-containing heterocyclic ring wherein the heterocyclic ring optionally contains one or two additional heteroatoms selected from the group consisting of N, O and S, or
(ii) a six-membered aromatic nitrogen-containing heterocyclic ring wherein the heterocyclic ring optionally contains one or two additional heteroatoms selected from the group consisting of N, O, and S; or
(iii) a six-membered non-aromatic nitrogen-containing heterocyclic ring wherein the heterocyclic ring optionally contains one or two additional heteroatoms selected from the group consisting of N and S;
   R⁸ is selected from the group consisting of hydrogen, C₁₋₄alkyl, haloC₁₋₄alkyl, and halogen;
   R⁹ is selected from the group consisting of hydrogen and halogen;
   R¹⁰ is selected from the group consisting of haloC₁₋₄alkyl, C₁₋₄alkyl, halogen, hydrogen, and C₁₋₄alkoxy;
   R¹¹ are independently selected from the group consisting of halogen, cyano, cyanoC₁₋₄alkyl, hydroxyl, oxo (═O), C₁₋₄alkyl optionally substituted with a five- or six-membered heterocyclic group containing 1 or 2 heteroatoms selected from the group consisting of O, N, and S, haloC₁₋₄alkyl, C₁₋₄alkoxy, hydroxylC₁₋₄alkyl, C₁₋₄alkoxyC₁₋₄alkyl, C₁₋₄alkylsulfone, amino, monoC₁₋₄alkylamino, diC₁₋₄alkylamino, aminoC₁₋₄alkyl, —C₁₋₄alkylene-C(═O)NH_{(2-q)}(C₁₋₆alkyl)_{q}), —C₁₋₄alkylene-NHC(═O)C₁₋₆alkyl, sulfonamide, sulfonamideC₁₋₄alkyl, 3 to 6-membered cycloalkyl, C₁₋₄alkyl substituted with 3 to 6-membered cycloalkyl, a five- or six-membered unsaturated heterocyclic group containing 1, 2, 3 or 4 heteroatoms selected from the group consisting of O, N, and S, and optionally substituted four- to six-membered saturated heterocyclic group containing 1 or 2 heteroatoms selected from the group consisting of O, N, and S where the optional substituent is selected from C₁₋₄alkyl;
   q is selected from the group consisting of 0, 1 and 2; and
   d is selected from the group consisting of 0, 1 and 2,
   and at least one additional compound having an antitumor effect, at least one tautomer thereof, at least one solvate thereof, or at least one pharmaceutically acceptable salt thereof selected from the group consisting of molecular targeted drugs and cytotoxic drugs.

### Item 42

The kit for malignant tumor treatment according to item 41, wherein the compound represented by formula (I), the tautomer thereof, the solvate thereof, or the pharmaceutically acceptable salt thereof is administered before, simultaneously with, or after administration of the at least one additional compound having an antitumor effect, the at least one tautomer thereof, the at least one solvate thereof, or the at least one pharmaceutically acceptable salt thereof.

### Item 43

The kit for malignant tumor treatment according to item 41 or 42, wherein the compound represented by formula (I) is selected from the group consisting of 2-((1R,2R,4S)-2-amino-7-azabicyclo[2.2.1]heptan-7-yl)-5-(3,4-dichloro-2-methyl-2H-indazol-5-yl)-3-methyl-3,7-dihydro-4H-pyrrolo[2,3-d]pyrimidin-4-one, 6-((1R,4R,7R)-7-amino-2-azabicyclo[2.2.1]heptan-2-yl)-3-(3,4-dichloro-2-methyl-2H-indazol-5-yl)-5-methyl-1,5-dihydro-4H-pyrazolo[3,4-d]pyrimidin-4-one, 2-((1R,4R,7R)-7-amino-2-azabicyclo[2.2.1]heptan-2-yl)-5-(4-chloro-2-ethyl-2H-indazol-5-yl)-3-methyl-3,7-dihydro-4H-pyrrolo[2,3-d]pyrimidin-4-one, 2-((1R,4R,7R)-7-amino-2-azabicyclo[2.2.1]heptan-2-yl)-5-(4-chloro-2-methyl-2H-indazol-5-yl)-3-methyl-3,7-dihydro-4H-pyrrolo[2,3-d]pyrimidin-4-one, 2-((1R,4R,7R)-7-amino-2-azabicyclo[2.2.1]heptan-2-yl)-5-(3,4-dichloro-2-methyl-2H-indazol-5-yl)-3-methyl-3,7-dihydro-4H-pyrrolo[2,3-d]pyrimidin-4-one, 2-((1R,2R,4S)-2-amino-7-azabicyclo[2.2.1]heptan-7-yl)-5-(3-chloro-4-fluoro-2-methyl-2H-indazol-5-yl)-3-methyl-3,7-dihydro-4H-pyrrolo[2,3-d]pyrimidin-4-one, and 2-((1R,2R,4S)-2-amino-7-azabicyclo[2.2.1]heptan-7-yl)-5-(4-chloro-2-ethyl-2H-indazol-5-yl)-3-methyl-3,7-dihydro-4H-pyrrolo[2,3-d]pyrimidin-4-one.

### Item 44

The kit for malignant tumor treatment according to any one of items 41 to 43, wherein the compound represented by formula (I) is 2-((1R,2R,4S)-2-amino-7-azabicyclo[2.2.1]heptan-7-yl)-5-(3,4-dichloro-2-methyl-2H-indazol-5-yl)-3-methyl-3,7-dihydro-4H-pyrrolo[2,3-d]pyrimidin-4-one.

### Item 45

The kit for malignant tumor treatment according to any one of items 41 to 44, wherein the at least one additional compound having an antitumor effect, the at least one tautomer thereof, the at least one solvate thereof, or the at least one pharmaceutically acceptable salt thereof is selected from the group consisting of a tyrosine kinase inhibitor, a RAS-MAPK pathway inhibitor, a PI3K pathway inhibitor, a BCL2 inhibitor, a CDK4/6 inhibitor, an HDAC inhibitor, a topoisomerase inhibitor (a topoisomerase I inhibitor, and topoisomerase II inhibitor), an alkylating agent, an anthracycline antibiotic, an alkaloid, an anti-metabolite, an anti-microtubule agent, a platinum-containing drug, a proteasome inhibitor, and a thalidomide analog drug.

### Item 46

The kit for malignant tumor treatment according to any one of items 41 to 45, wherein the molecular targeted drug is selected from the group consisting of an AKT inhibitor, ALK inhibitor, Bcl2 inhibitor, BCR-ABL inhibitor, BRAF inhibitor, CDK4/6 inhibitor, CDK inhibitor, EGFR inhibitor, Erk1/2 inhibitor, FGFR inhibitor, FLT3 inhibitor, HER2 inhibitor, MEK inhibitor, Multi-kinase inhibitor, PI3K inhibitor, RAF inhibitor, HDAC inhibitor, topoisomerase I inhibitor, topoisomerase II inhibitor, alkylating agent, anthracycline antibiotics, alkaloid, anti-metabolite, anti-microtubule agent, platinum-containing drug, and proteasome inhibitor.

### Item 47

The kit for malignant tumor treatment according to any one of items 41 to 46, wherein the molecular targeted drug is selected from the group consisting of cetuximab, MK-2206, alectinib, crizotinib, venetoclax, imatinib, dasatinib, ponatinib, dabrafenib, vemurafenib, sorafenib, palbociclib, abemaciclib, osimertinib, gefitinib, erlotinib, afatinib, brigatinib, ulixertinib, (2R)-2-(6-{5-chloro-2-[(oxan-4-yl)amino]pyrimidin-4-yl}-1-oxo-2,3-dihydro-1H-isoindol-2-yl)-N-[(1S)-1-(3-fluoro-5-methoxyphenyl)-2-hydroxyethyl]propanamide or a salt thereof, erdafitinib, giltertinib, lapatinib, neratinib, trametinib, cobimetinib, binimetinib, regorafenib, sunitinib, nintedanib, anlotinib, vandetanib, lenvatinib, alpelisib and idelalisib/CAL-101, vorinostat (SAHA), irinotecan (SN-38), etoposide, cyclophosphamide, doxorubicin, gemcitabine, pemetrexed, 5-FU, FdUrd, FTD, paclitaxel, cisplatin, oxaliplatin, bortezomib, afuresertib, trans-3-amino-1-methyl-3-(4-(3-phenyl-5H-imidazo[1,2-c]pyrido[3,4-e][1,3]oxazin-2-yl)phenyl)cyclobutanol, capivasertib, ipatasertib, triciribine, miransertib, lorlatinib, ceritinib, repotrectinib, ensartinib, alkotinib, WX-0593, SAF-189s, CT-707, TQ-B3101, sabutoclax, apogossypol, obatoclax, navitoclax, APG-2575, APG-1252, asciminib, olverembatinib, encorafenib, lifirafenib, LXH-254, ribociclib, lerociclib, trilaciclib, alvocidib, GLR-2007, SHR-6390, XZP-3287, BPI-1178, PF-06873600, NUV-422, FCN-437, seliciclib, mevociclib, milciclib, fadraciclib, zotiraciclib, dinaciclib, samuraciclib, voruciclib, FIT-039, PF-07104091, BEY-1107, panitumumab, sutetinib, allitinib, epitinib, xiliertinib, rociletinib, dacomitinib, simotinib, olmutinib, yinlitinib, mefatinib, alflutinib, almonertinib, icotinib, naquotinib, poziotinib, epertinib, sapitinib, cipatinib, tarloxotinib, pyrotinib, pirotinib, lazertinib, varlitinib, tesevatinib, canertinib, mobocertinib, duligotuzumab, olafertinib, zorifertinib, pelitinib, DZD-9008, ASK-120067, BPI-7711, QLNC-120, ametumumab, imgatuzumab, amivantamab, seribantumab, nimotuzumab, serclutamab, depatuxizumab, tomuzotuximab, SCT-200, ravoxertinib, LY3214996, MK-8353, LTT462, HH-2710, infigratinib, pemigatinib, orantinib, derazantinib, roblitinib, rogaratinib, zoligratinib, E-7090, AZD-4547, ODM-203, ICP-192, HMPL-453, bemarituzumab, quizartinib, crenolanib, flysyn, mivavotinib, PHI-101, MEN-1703, FF-10101, HM-43239, E-6201, ENMD-2076, larotinib, tucatinib (irbinitinib), BDTX-189, trastuzumab, pertuzumab, zanidatamab, zenocutuzumab, margetuximab, KN-026, BAT-8001, TAA-013, KL-A166, selumetinib, refametinib, mirdametinib, pimasertib, HL-085, NFX-179, nilotinib, dovitinib, axitinib, vatalinib, pazopanib, avapritinib, famitinib, catequentinib, necuparanib, surufatinib, lucitanib, midostaurin, vorolanib, bevasiranib, bevacizumab, ranibizumab, vanucizumab, navicixizumab, ramucirumab, BAT-5906, VGX-100, CSL-346, duvelisib, copanlisib, buparlisib, paxalisib, voxtalisib, zandelisib, dezapelisib, linperlisib, inavolisib, parsaclisib, eganelisib, nemiralisib, seletalisib, gedatolisib, leniolisib, tenalisib, pictilisib, bimiralisib, BBP-681, BGB-10188, MEN-1611, ASN-003, ACP-319, panobinostat, resminostat, abexinostat, romidepsin, belinostat, entinostat, quisinostat, pracinostat, tefinostat, mocetinostat, givinostat, dacinostat, ivaltinostat, domatinostat, fimepinostat, tinostamustine, Remetinostat, tucidinostat, ricolinostat, CXD-101, REC-2282, veltuzumab, rituximab, ublituximab, nogitecan, simmitecan, gimatecan, topotecan, cositecan, belotecan, govitecan, deruxtecan, AR-67, camsirubicin, Aldoxorubicin, vosaroxin, mitoxantrone, evofosfamide, amrubicin, sobuzoxane, epirubicin, F-14512, dacarbazine, temozolomide, nimustine, busulfan, procarbazine, melphalan, mitomycin C, daunorubicin, vincristine, vinblastine, vinorelbine, eribulin, trifluridine, docetaxel, cabazitaxel, avanbulin, fluorapacin, mertansine, carboplatin, nedaplatin, ixazomib, marizomib, carfilzomib and LXE-408.

### Item 48

The kit for malignant tumor treatment according to any one of items 41 to 47, wherein the malignant tumor is selected from the group consisting of epithelial cancer (respiratory cancer, gastrointestinal cancer, genital cancer, cancer of the secretory system, breast cancer, etc.), mesothelioma, sarcoma, hematopoietic tumor, tumors of the central nervous system, retinoblastoma, and tumors of the peripheral nervous system.

### Advantageous Effects of Invention

According to the present invention, cancer treatment that exerts high antitumor effects (particularly, a cytoreductive effect and a tumor growth-delaying effect (life-prolonging effect)), while suppressing the occurrence of side effects of an antitumor agent, can be performed. Therefore, the long-term survival of cancer patients can be brought about.

### BRIEF DESCRIPTION OF DRAWINGS

FIG. 1A

Anti-tumor effects of Compound 1 and cetuximab used alone or concomitantly.

FIG. 1B

Anti-tumor effects of Compound 1 and cetuximab used alone or concomitantly.

FIG. 2

Anti-tumor effects of Compound 1 and Compound 9 in vivo used alone or concomitantly.

FIG. 3

Anti-tumor effects of Compound 1 and Compound 9 in vivo used alone or concomitantly.

### DESCRIPTION OF EMBODIMENTS

According to the first embodiment, the present invention provides a combination drug for the treatment of a malignant tumor comprising a compound represented by formula (I), or a tautomer thereof, a solvate thereof, or a pharmaceutically acceptable salt thereof, and at least one additional compound having an antitumor effect, at least one tautomer thereof, at least one solvate thereof, or at least one pharmaceutically acceptable salt thereof. The compound represented by formula (I) or a pharmaceutically acceptable salt thereof of the present invention is a novel pyrrolopyrimidone or pyrazolopyrimidone compound comprising

(i) a monocyclic, bicyclic, bridged cyclic or spirocyclic nitrogen-containing saturated five- to seven-membered heterocyclic group and

(ii) an aromatic or non-aromatic fused ring containing a benzo-ring, and a five- or six-membered nitrogen containing heterocyclic ring.

In the present specification, “*” represents a bonding position, unless otherwise specified.

In the present specification, examples of the “halogen” include fluorine, chlorine, bromine, iodine, and the like, with fluorine, chlorine, bromine, or iodine being preferable, and fluorine or chlorine being more preferable.

In the present specification, the “alkyl” may be straight or branched. Examples of C₁₋₆alkyl include methyl, ethyl, n-propyl, isopropyl, n-butyl, isobutyl, sec-butyl, tert-butyl, n-pentyl, isopentyl, tert-pentyl, and n-hexyl. Examples of C₁₋₄alkyl include methyl, ethyl, n-propyl, isopropyl, n-butyl, isobutyl, sec-butyl, tert-butyl.

In the present specification, “alkylene” is a divalent group where one hydrogen is removed from above-listed alkyl groups. Examples of C₁₋₄alkylene include straight C₁₋₄alkylene such as methylene, ethylene, propylene, butylene, and branched C₁₋₄alkylene such as

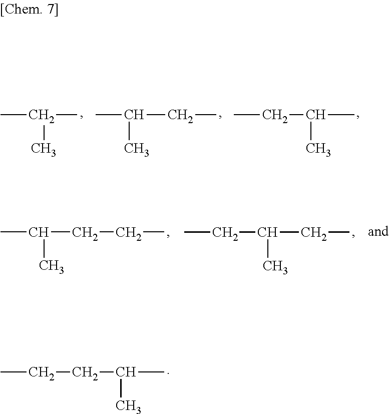

In the present specification, “heterocyclic ring” includes any monocyclic or polycyclic, saturated or unsaturated ring system comprising carbon atoms and at least one hetero atom. “heterocyclic ring” covers aromatic and non-aromatic groups.

In the present specification, examples of “C₂₋₃alkenylene” include vinylene and allylene.

In the present specification, the “3 to 6-membered cycloalkyl” includes cyclopropyl, cyclobutyl, cyclopentyl and cyclohexyl.

In the present specification, “aminoC₁₋₄alkyl” is the above-listed straight or branched C₁₋₄ alkyl having one amino group and refers to a group represented by —C₁₋₄alkylene-NH₂. Examples include -methylene-amino, -ethylene-amino, -propylene-amino, -butylene-amino, and the like.

Examples of “monoC₁₋₄alkylamino” include amino monosubstituted with straight or branched C₁₋₄alkyl, such as methylamino, ethylamino, n-propylamino, isopropylamino, n-butylamino, isobutylamino, tert-butylamino, and the like.

Examples of “diC₁₋₄alkylamino” include amino disubstituted with the same or different straight or branched C₁₋₄alkyl groups, such as dimethylamino, diethylamino, di(n-propyl)amino, diisopropylamino, di(n-butyl)amino, diisobutylamino, di(tert-butyl)amino, and the like.

In the present specification, examples of the “hydroxyC₁₋₄alkyl” include the above-listed straight or branched alkyl groups that have at least one hydroxy group (e.g., one or two hydroxy groups). Specific examples include hydroxymethyl, 2-hydroxyethyl, 1-hydroxyethyl, 3-hydroxypropyl, 2-hydroxypropyl, 1-methyl-2-hydroxyethyl, 4-hydroxybutyl, 2,2-dimethyl-2-hydroxyethyl, and the like, with hydroxyalkyl having one hydroxy group being preferable.

In the present specification, the “C₁₋₄alkoxy” refers to oxy(—O—) to which the above-listed straight or branched C₁₋₄alkyl is bonded. Examples include methoxy, ethoxy, n-propoxy, isopropoxy, n-butoxy, isobutoxy, tert-butoxy etc.

In the present specification, examples of the “cyanoC₁₋₄alkyl” include the above-listed straight or branched C₁₋₄alkyl groups that have at least one cyano group (e.g., one or two cyano groups). Specific examples include cyanomethyl, 2-cyanoethyl, 1-cyanoethyl, 3-cyanopropyl, 2-cyanopropyl, 1-methyl-2-cyanoethyl, 4-cyanobutyl, 2,2-dimethyl-2-cyanoethyl, and the like, with cyanoalkyl having one cyano group being preferable.

In the present specification, the “haloC₁₋₄alkyl” is the above-listed straight or branched C₁₋₄ alkyl having 1 to 7 halogen atoms (halogeno C₁₋₄alkyl). Examples include fluoromethyl, difluoromethyl, trifluoromethyl, trichloromethyl, fluoroethyl, 1,1,1-trifluoroethyl, monofluoro-n-propyl, perfluoro-n-propyl, and perfluoroisopropyl.

In the present specification, “C₁₋₄alkoxyC₁₋₄alkyl” is the above-listed straight or branched C₁₋₄ having one of the above listed C₁₋₄alkoxy and refers to a group represented by —C₁₋₄alkylene-C₁₋₄alkoxy (—C₁₋₄alkylene-O—C₁₋₄alkyl). Examples of C1-4alkylene, C₁₋₄alkoxy and C₁₋₄alkyl are above listed.

In the present specification, “C₁₋₄alkylsulfone” refers to a group represented by —SO₂—C₁₋₄alkyl. Examples include methylsulfone, ethylsulfone, propylsulfone, butylsulfone, and the like.

In the present specification, examples of “—C₁₋₄alkylene-C(═O)NH_{(2-q)}(C₁₋₆alkyl)_{q})” wherein q is an integer of 0, 1 or 2, include —C₁₋₄alkylene-C(═O)NH₂, —C₁₋₄alkylene-C(═O)NH(C₁₋₆alkyl), and —C₁₋₄alkylene-C(═O)N(C₁₋₆alkyl) 2. Examples of C₁₋₄alkylene and C₁₋₆alkyl are above listed.

In the present specification, “—C₁₋₄alkylene-NHC(═O)C₁₋₆alkyl,” refers to a group where the above-mentioned C₁₋₄alkylene and C₁₋₆alkyl, are joined by an amide bond (—NHC(═O)—). Examples of C₁₋₄alkylene and C₁₋₆alkyl are above listed.

In the present specification, “sulfonamideC₁₋₄alkyl” refers to a group represented by —C₁₋₄alkylene-SO₂—NH₂. Examples include —SO₂—NH₂, -methylene-SO₂—NH₂, -ethylene-SO₂—NH₂, -propylene-SO₂—NH₂, -butylene-SO₂—NH₂, and the like.

In the compound represented by formula (I) of the present invention, X represents CH or N. When X represents CH, the compound represented by formula (I) is a pyrrolopyrimidone compound, and when X represents N, the compound represented by formula (I) is a pyrazolopyrimidone compound.

In the compound represented by formula (I) of the present invention, R¹ represents methyl (—CH₃).

In the compound represented by formula (I) of the present invention, the following portion (hereafter referred to as portion Z):

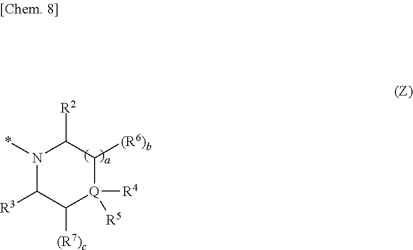

wherein Q, R², R³, R⁴, R⁵, R⁶ and R⁷, a, b and c are as defined above;
is a monocyclic, bicyclic, bridged cyclic or spirocyclic nitrogen-containing saturated heterocyclic group.

In the compound represented by formula (I) of the present invention, R² and R³ independently represent any one selected from the group consisting of hydrogen and C₁₋₄alkyl.

In the compound represented by formula (I) of the present invention, R⁶ and R⁷ independently represent any one selected from the group consisting of halogen, C₁₋₄alkyl, hydroxyC₁₋₄alkyl, and hydroxyl. When Q is N, then R⁶ or R⁷ do not represent halogen or hydroxyl, thus represents C₁₋₄alkyl.

In the compound represented by formula (I) of the present invention, Q represents C or N.

In one embodiment when Q represents C, R⁴ is amino, aminoC₁₋₄alkyl or monoC₁₋₄alkylamino;

R⁵ is hydrogen, C₁₋₄alkyl, halogen, hydroxyC₁₋₄alkyl, C₁₋₄alkoxy, haloC₁₋₄alkyl or C₁₋₄alkoxyC₁₋₄alkyl. In one embodiment when R⁴ is amino then R³ is selected from the group consisting of hydrogen, C₁₋₄alkyl, hydroxyC₁₋₄alkyl, C₁₋₄alkoxy, haloC₁₋₄alkyl and C₁₋₄alkoxyC₁₋₄alkyl.

In such embodiment, the portion Z is a monocyclic nitrogen-containing saturated five to seven-membered heterocyclic group containing one nitrogen, represented by the formula below:

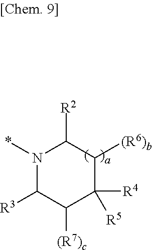

wherein R², R³, R⁴, R⁵, R⁶ and R⁷, a, b and c are as defined above;

In another embodiment when Q represents C, R⁴ and R³ together with Q form a four- to six-membered ring that can optionally contain 1 to 3 heteroatoms or groups independently selected from the group consisting of N, O, S, NH, C(O) and S(O)ₘ, and said ring formed by R⁴ and R³ can be unsubstituted or substituted with 1 to 4 groups independently selected from the group consisting of amino, halogen, haloC₁₋₄alkyl, hydroxyl, methoxy, methylamino, and C₁₋₄alkyl, and m is selected from 1 or 2.

In such embodiment, the portion Z is a spirocyclic nitrogen-containing saturated heterocyclic group containing eight to twelve members including Q, one to four among the members being nitrogen, and one to four among the members optionally being identical or different heteroatoms selected from the group consisting of oxygen, and sulfur. In such embodiment, the portion Z is represented be represented by the formula below:

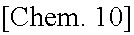

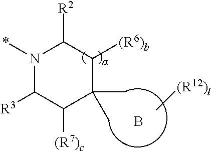

wherein R², R³, a, b and c are as defined above;
wherein Ring B is a saturated four- to six-membered ring that can optionally contain 1 to 3 heteroatoms or groups independently selected from the group consisting of N, O, S, NH, C(O) and S(O)m,
R¹² is independently selected from the group consisting of amino, halogen, haloC₁₋₄alkyl, hydroxyl, methoxy, methylamino, and C₁₋₄alkyl,
l is a integer selected from the group consisting of 0, 1, 2, 3 and 4,
m is a integer selected from the group consisting of 1 and 2.

Examples of the four- to six-membered ring that can optionally contain 1 to 3 heteroatoms or groups independently selected from the group consisting of N, O, C(O) and S(O)ₘ include:

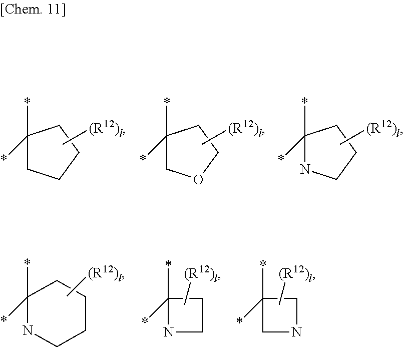

wherein R¹² is as defined above.

In one embodiment when Q represents N, then R⁴ is absent and R³ is hydrogen.

In such embodiment, the portion Z may be represented by the formula below:

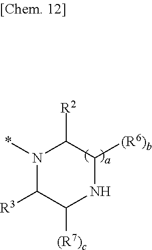

wherein R², R³, a, b and c are as defined above; R⁶ and R⁷ independently selected from the group consisting of hydroxyC₁₋₄alkyl and C₁₋₄alkyl, provided a is not zero;
and is a monocyclic nitrogen-containing saturated five to seven-membered heterocyclic group containing two nitrogen.

In the compound represented by formula (I) of the present invention, R², R³, R⁶ and R⁷ may alternatively have the following structure wherein any two groups selected from the group consisting of R², R³, R⁶ and R⁷ together form a one- to three-membered bridge group selected from the group consisting of C₁₋₃alkylene, C₂₋₃alkenylene, methylene-NRq-methylene and methylene-O-methylene, wherein the bridge group is optionally substituted with a group selected from the group consisting of C₁₋₄alkyl, hydroxyl and halogen and Rq is selected from the group consisting of hydrogen, and C₁₋₄alkyl.

Examples of such embodiment includes the portion Z being represented by any one of the formulas below:

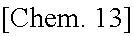

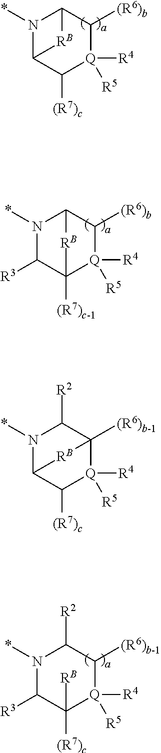

wherein Q, R², R³, R⁴, R⁵, R⁶ and R⁷, a, b and c are as defined above,
R^{B} represents a one- to three-membered bridge group selected from the group consisting of straight C₁₋₃alkylene, C₂₋₃alkenylene, methylene-NR^{q}-methylene and methylene-O-methylene, wherein the bridge group is optionally substituted with a group selected from the group consisting of C₁₋₄alkyl, hydroxyl and halogen and R^{q} is selected from the group consisting of hydrogen and C₁₋₄alkyl.

In the compound represented by formula (I) of the present invention, Q is C, c is 2, R⁴ is hydrogen and the two R⁷ join to form a 4 to 6-membered nitrogen containing ring. Examples of such embodiment includes the portion Z being represented by any one of the formulas below:

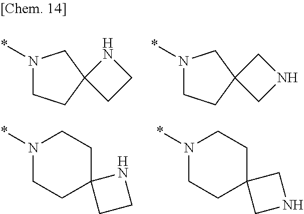

In the compound represented by formula (I) of the present invention, R⁴ and R⁷ may alternatively form a four- to six-membered ring containing one N atom. Examples of such embodiment where includes the portion Z being represented by any one of the formulas below:

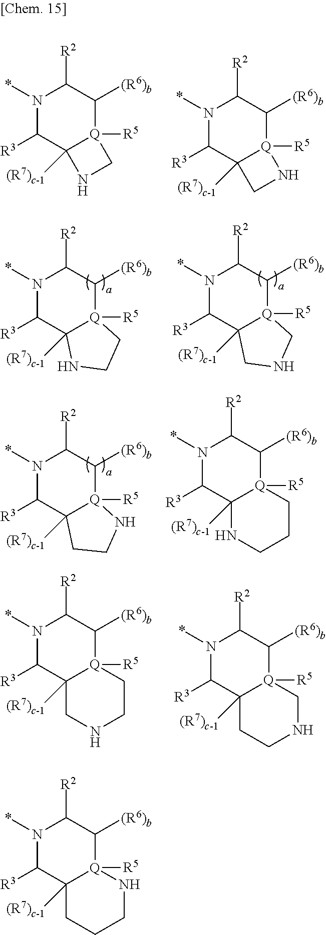

wherein Q, R², R³, R⁴, R⁵, R⁶ and R⁷, a, b and c are as defined above.

In the compound represented by formula (I) of the present invention, R³ and R⁷ may alternatively form a three- to six-membered ring. Examples of such embodiment includes the portion Z being represented by any one of the formulas below:

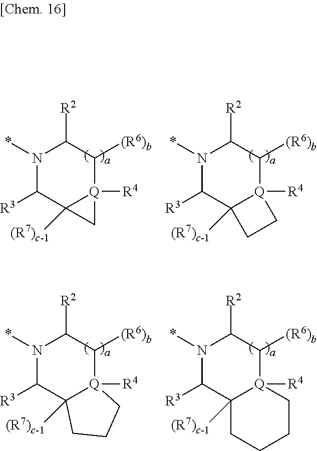

wherein R², R³, R⁴, R⁵, R⁶ and R⁷, a, b and c are as defined above;

In one alternative embodiment, R⁶ and R⁷ alternatively form a direct bond. Examples of such embodiment includes the portion Z being represented by formula below:

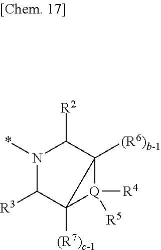

wherein R², R³, R⁴, R⁵, R⁶, R⁷, b and c are as defined above.

In the compound represented by formula (I), a is an integer selected from the group consisting of 0, 1 and 2.

In the compound represented by formula (I), b is an integer selected from the group consisting of 0, 1 and 2.

In the compound represented by formula (I), c is an integer selected from the group consisting of 0, 1 and 2;

Preferable embodiments include the portion Z being represented by any one of the formulas below:

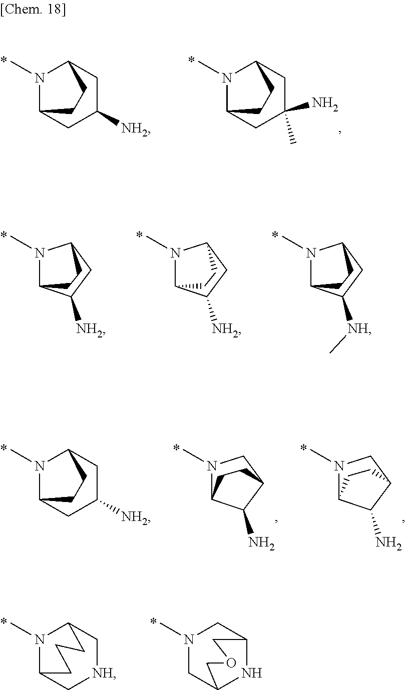

More preferable embodiments include the portion Z being represented by any one of the formulas below:

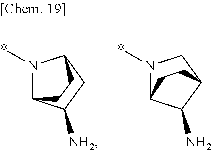 [SMILES: *N1[C@H]2CC[C@@H]1[C@H](N)C2 ; *N1C[C@H]2CC[C@@H]1[C@@H]2N]

In the compound represented by formula (I) of the present invention, the following portion (hereafter referred to as portion Y):

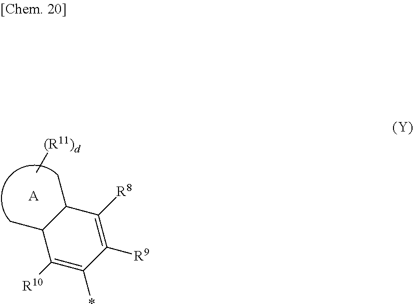

wherein Ring A, R⁸ R⁹, R¹⁰, R¹¹ and d are as defined above; is a an aromatic or non-aromatic fused ring containing a benzo-ring, and a five or six-membered nitrogen containing heterocyclic ring.

In the compound represented by formula (I), Ring A represented below:

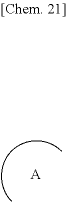

forms, together with the benzo-ring to which this group is bonded, a five or six-membered nitrogen containing heterocyclic ring.

Specifically, Ring A is either:

(i) a five-membered nitrogen-containing heterocyclic ring wherein the heterocyclic ring optionally contains one or two additional heteroatoms selected from the group consisting of N, O and S, or
(ii) a six-membered aromatic nitrogen-containing heterocyclic ring, wherein the heterocyclic ring optionally contains one or two additional heteroatoms selected from the group consisting of N, O and S; or
(iii) a six-membered non-aromatic nitrogen-containing heterocyclic ring, wherein the heterocyclic ring optionally contains one or two additional heteroatoms selected from the group consisting of N and S.

The five-membered nitrogen-containing heterocyclic ring wherein the heterocyclic ring optionally contains one or two additional heteroatoms selected from the group consisting of N, O and S may be an five-membered aromatic nitrogen-containing heterocyclic ring or a five-membered non-aromatic nitrogen-containing heterocyclic ring. Such heterocyclic ring contains two to four carbon atoms including the two carbon atoms that is shared with the benzo-ring to which this group is bonded, one to three nitrogen atoms, and the carbon atoms that is not shared with the benzo ring (one or two carbon atoms) replaced with an oxygen atom or a sulfur atom.

Examples of five-membered aromatic nitrogen-containing heterocyclic rings include pyrrole, imidazole, pyrazole, oxazole, isoxazole, thiazole, isothiazole, and the like.

Examples of five-membered non-aromatic nitrogen-containing heterocyclic rings include pyrrolidine, pyrazolidine, triazolidine, oxazolidine, isoxazolidine, thiazolidine, isothiazolidine, and the like.

The six-membered aromatic nitrogen-containing heterocyclic ring, wherein the heterocyclic ring optionally contains one or two additional heteroatoms selected from the group consisting of N, O and S. Such heterocyclic ring contains two to five carbon atoms including the two carbon atoms that is shared with the benzo-ring to which this group is bonded, one to three nitrogen atoms, and the carbon atoms that is not shared with the benzo-ring (one, two or three carbon atoms) replaced with an oxygen atom or a sulfur atom.

Examples of six-membered aromatic nitrogen-containing heterocyclic ring include pyridine, pyrazine, pyrimidine, pyridazine, triazine, oxazine, thiazine, and the like.

The six-membered non-aromatic nitrogen-containing heterocyclic ring, wherein the heterocyclic ring optionally contains one or two additional heteroatoms selected from the group consisting of N and S. Such heterocyclic ring contains two to five carbon atoms including the two carbon atoms that is shared with the benzo-ring to which this group is bonded, one to three nitrogen atoms, and the carbon atoms that is not shared with the benzo-ring (one, two or three carbon atoms) replaced with a sulfur atom.

Examples of six-membered non-aromatic nitrogen-containing heterocyclic ring include piperidine, piperazine, morpholine, and the like.

In the compound represented by formula (I), R⁸ represents one selected from the group consisting of hydrogen, C₁₋₄alkyl, haloC₁₋₄alkyl and halogen.

In the compound represented by formula (I), R⁹ represents one selected from the group consisting of hydrogen and halogen.

In the compound represented by formula (I), R¹⁰ represents one selected from the group consisting of haloC₁₋₄alkyl, C₁₋₄alkyl, halogen, hydrogen and C₁₋₄alkoxy.

In the compound represented by formula (I), each R¹¹ independently represents one selected from the group consisting of halogen, cyano, cyanoC₁₋₄alkyl, hydroxyl, oxo (═O), C₁₋₄alkyl optionally substituted with five- or six-membered heterocyclic group containing 1 or 2 heteroatoms selected from O, N, or S, haloC₁₋₄alkyl, C₁₋₄alkoxy, hydroxylC₁₋₄alkyl, C₁₋₄alkoxyC₁₋₄alkyl, C₁₋₄alkylsulfone, amino, monoC₁₋₄alkylamino, diC₁₋₄alkylamino, aminoC₁₋₄alkyl, —C₁₋₄alkylene-C(═O)NH_{(2-q)}(C₁₋₆alkyl)_{q}), —C₁₋₄alkylene-NHC(═O)C₁₋₆alkyl, sulfonamide, sulfonamideC₁₋₄alkyl, 3 to 6-membered cycloalkyl, C₁₋₄alkyl substituted with 3 to 6-membered cycloalkyl, five- or six-membered unsaturated heterocyclic group containing 1, 2, 3 or 4 heteroatoms selected from O, N, or S, and optionally substituted four- to six-membered saturated heterocyclic group containing 1 or 2 heteroatoms selected from O, N, or S where the optional substituent is selected from C₁₋₄alkyl.

When R¹¹ is oxo (═O), the atomic bonding between R¹¹ and Ring A is a double bond. In other cases where R¹¹ is a monovalent group, the atomic bonding between R¹¹ and Ring A is a single bond.

In the compound represented by formula (I), q is an integer selected from the group consisting of 0, 1 and 2.

In the compound represented by formula (I), d is an integer selected from the group consisting of 0, 1 and 2.

Preferable embodiments include the portion Y being represented by any one of the formulas below:

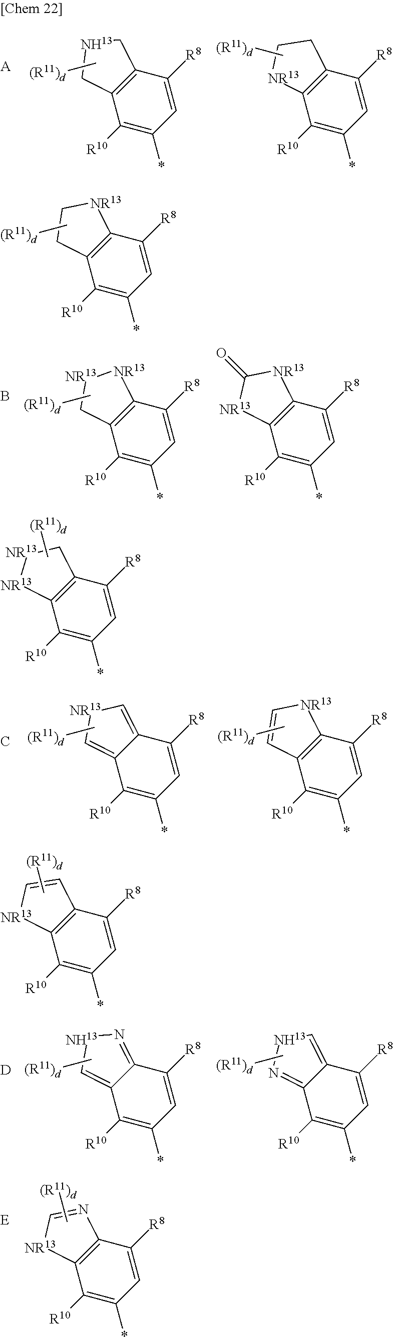

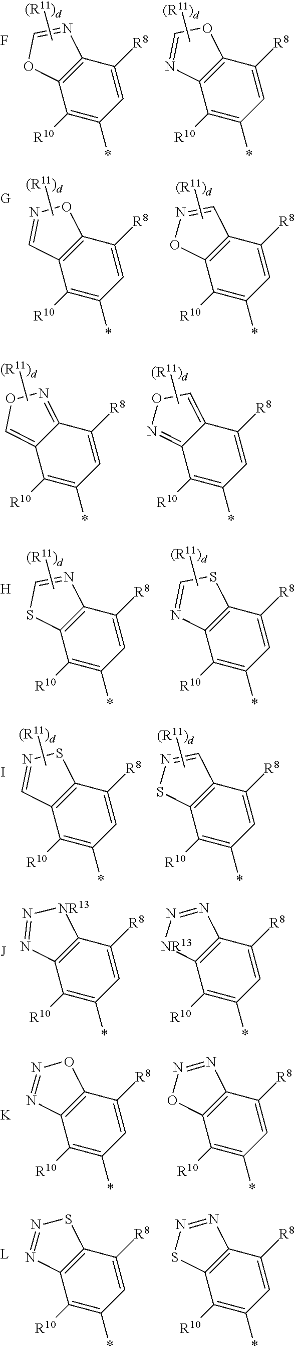

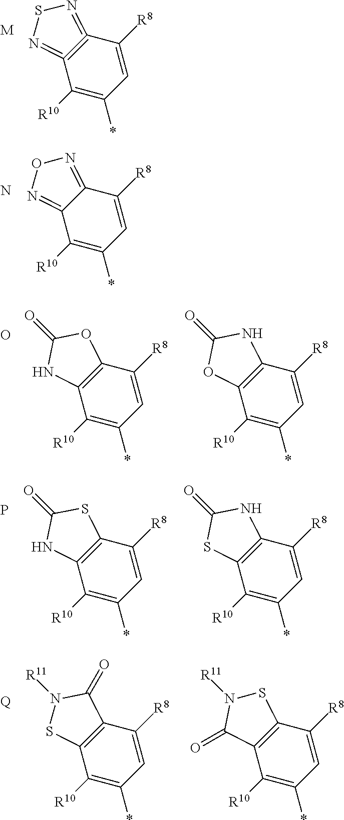

wherein R, R¹⁰, R¹¹ and d are as defined above;
R¹³s are independently selected from the group consisting of hydrogen, cyano, cyanoC₁₋₄alkyl, C₁₋₄alkyl optionally substituted with five- or six-membered heterocyclic group containing 1 or 2 heteroatoms selected from O, N, or S, haloC₁₋₄alkyl, C₁₋₄alkoxy, hydroxylC₁₋₄alkyl, C₁₋₄alkoxyC₁₋₄alkyl, C₁₋₄alkylsulfone, aminoC₂₋₄alkyl, —C₁₋₄alkylene-C(═O)NH_{(2-q)} (C₁₋₆alkyl)_{q}), —C₁₋₄alkylene-NHC(═O)C₁₋₆alkyl, sulfoneamideC₁₋₄alkyl, 3 to 6-membered cycloalkyl, C₁₋₄alkyl substituted with 3 to 6-membered cycloalkyl, five- or six-membered unsaturated heterocyclic group containing 1, 2, 3 or 4 heteroatoms selected from O, N, or S, and optionally substituted four- to six-membered saturated heterocyclic group containing 1 or 2 heteroatoms selected from O, N, or S where the optional substituent is selected from C₁₋₄alky.

Other preferable embodiments include the portion Y being represented by any one of the formulas below:

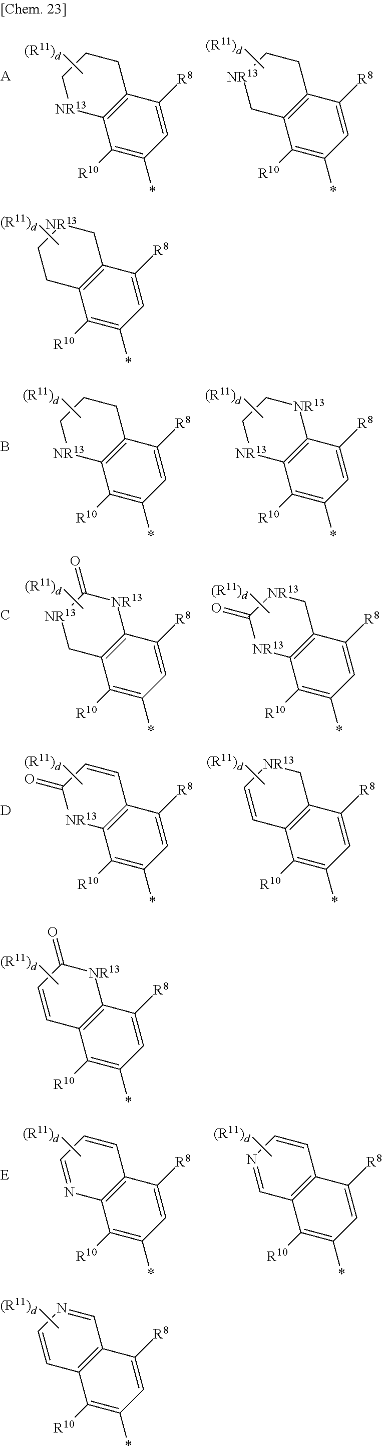

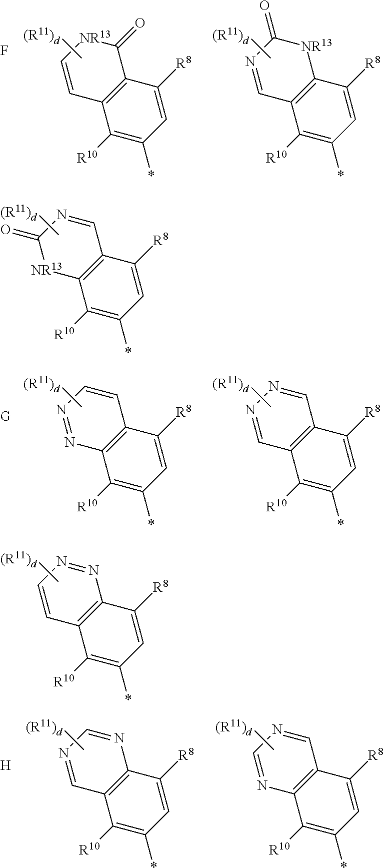

wherein R¹, R¹⁰, R¹¹ and d are as defined above;
R¹³s are independently selected from the group consisting of hydrogen, cyano, cyanoC₁₋₄alkyl, C₁₋₄alkyl optionally substituted with five- or six-membered heterocyclic group containing 1 or 2 heteroatoms selected from O, N, or S, haloC₁₋₄alkyl, C₁₋₄alkoxy, hydroxylC₁₋₄alkyl, C₁₋₄alkoxyC₁₋₄alkyl, C₁₋₄alkylsulfone, aminoC₂₋₄alkyl, —C₁₋₄alkylene-C(═O)NH_{(2-q)} (C₁₋₆alkyl)_{q}), —C₁₋₄alkylene-NHC(═O)C₁₋₆alkyl, sulfoneamideC₁₋₄alkyl, 3 to 6-membered cycloalkyl, C₁₋₄alkyl substituted with 3 to 6-membered cycloalkyl, five- or six-membered unsaturated heterocyclic group containing 1, 2, 3 or 4 heteroatoms selected from O, N, or S, and optionally substituted four- to six-membered saturated heterocyclic group containing 1 or 2 heteroatoms selected from O, N, or S where the optional substituent is selected from C₁₋₄alky.

More preferable embodiments include the portion Y being represented by any one of the formulas below:

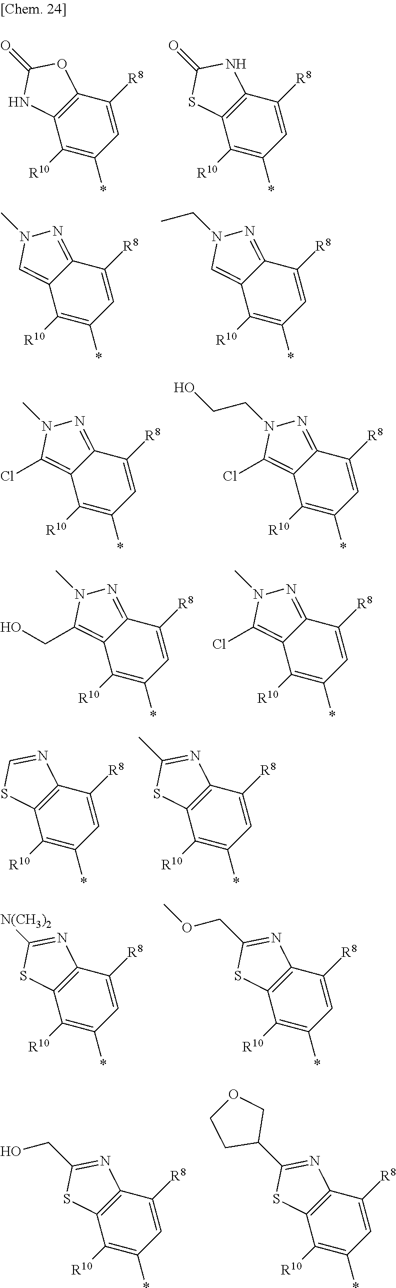

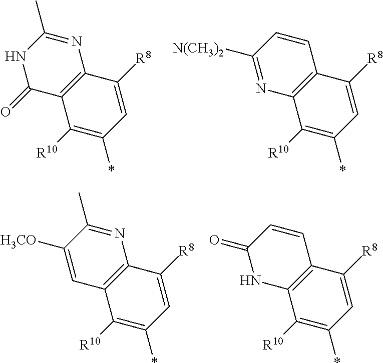

wherein R⁸ and R¹⁰ are as defined above.

More preferable embodiments include the portion Y being represented by any one of the formulas below:

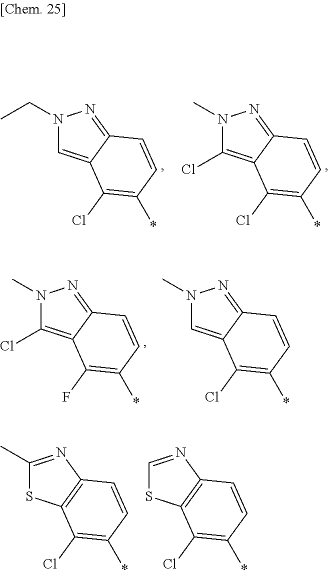

Particularly preferable embodiments include the portion Y being represented by any one of the formulas below:

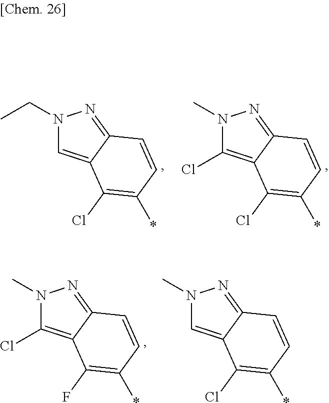

Formula (I) is preferably a compound by Formula (II)

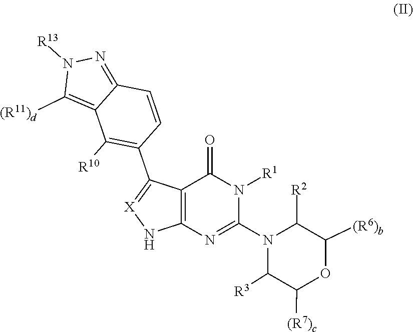

or a tautomer or a solvate or a pharmaceutically acceptable salt thereof, wherein:
the following portion:

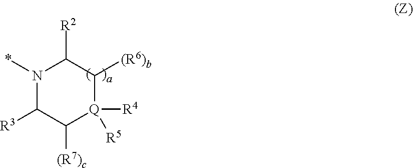

is selected the group consisting of

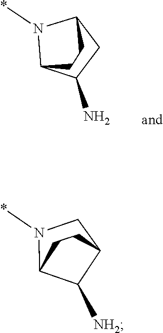 [SMILES: *N1[C@H]2CC[C@@H]1[C@H](N)C2 ; "and" ; *N1C[C@H]2CC[C@@H]1[C@@H]2N]

### X is CH or N;

R¹ is —CH₃;
R¹⁰ is selected from fluorine, chlorine or hydrogen;
R¹¹ is selected from fluorine or chlorine;
R¹³ is C₁₋₄alkyl;
d is selected from the group consisting of 0 and 1.

The following are examples of preferable compounds of the present invention:
2-(3,8-diazabicyclo[3.2.1]octan-8-yl)-5-(4-chloro-2-methyl-2H-indazol-5-yl)-3-methyl-3,7-dihydro-4H-pyrrolo[2,3-d]pyrimidin-4-one,
2-(4-(aminomethyl)-4-methylpiperidin-1-yl)-5-(4-chloro-2-ethyl-2H-indazol-5-yl)-3-methyl-3,7-dihydro-4H-pyrrolo[2,3-d]pyrimidin-4-one,
2-(4-amino-4-methylpiperidin-1-yl)-5-(4-chloro-2-ethyl-2H-indazol-5-yl)-3-methyl-3,7-dihydro-4H-pyrrolo[2,3-d]pyrimidin-4-one,
2-(exo-3-amino-8-azabicyclo[3.2.1]octan-8-yl)-5-(4-chloro-2-ethyl-2H-indazol-5-yl)-3-methyl-3,7-dihydro-4H-pyrrolo[2,3-d]pyrimidin-4-one,
2-(endo-3-amino-8-azabicyclo[3.2.1]octan-8-yl)-5-(4-chloro-2-ethyl-2H-indazol-5-yl)-3-methyl-3,7-dihydro-4H-pyrrolo[2,3-d]pyrimidin-4-one,
2-(endo-3-amino-8-azabicyclo[3.2.1]octan-8-yl)-5-(7-chloro-2-ethylbenzo[d]thiazol-6-yl)-3-methyl-3,7-dihydro-4H-pyrrolo[2,3-d]pyrimidin-4-one,
2-(endo-3-amino-8-azabicyclo[3.2.1]octan-8-yl)-5-(7-chloro-2-methylbenzo[d]thiazol-6-yl)-3-methyl-3,7-dihydro-4H-pyrrolo[2,3-d]pyrimidin-4-one,
2-(endo-3-amino-8-azabicyclo[3.2.1]octan-8-yl)-5-(4-chloro-2-methyl-2H-indazol-5-yl)-3-methyl-3,7-dihydro-4H-pyrrolo[2,3-d]pyrimidin-4-one,
2-(endo-3-Amino-8-azabicyclo[3.2.1]octan-8-yl)-5-(2-(tert-butyl)-4-chloro-2H-indazol-5-yl)-3-methyl-3,7-dihydro-4H-pyrrolo[2,3-d]pyrimidin-4-one,
2-(exo-3-amino-8-azabicyclo[3.2.1]octan-8-yl)-5-(4-chloro-2-methyl-2H-indazol-5-yl)-3-methyl-3,7-dihydro-4H-pyrrolo[2,3-d]pyrimidin-4-one,
(R)-2-(1-amino-8-azaspiro[4.5]decan-8-yl)-5-(4-chloro-2-ethyl-2H-indazol-5-yl)-3-methyl-3,7-dihydro-4H-pyrrolo[2,3-d]pyrimidin-4-one,
(S)-2-(4-amino-2-oxa-8-azaspiro[4.5]decan-8-yl)-5-(4-chloro-2-ethyl-2H-indazol-5-yl)-3-methyl-3,7-dihydro-4H-pyrrolo[2,3-d]pyrimidin-4-one,
5-(4-chloro-2-ethyl-2H-indazol-5-yl)-3-methyl-2-(endo-3-(methylamino)-8-azabicyclo[3.2.1]octan-8-yl)-3,7-dihydro-4H-pyrrolo[2,3-d]pyrimidin-4-one,
5-(4-chloro-2-methyl-2H-indazol-5-yl)-3-methyl-2-(endo-3-(methylamino)-8-azabicyclo[3.2.1]octan-8-yl)-3,7-dihydro-4H-pyrrolo[2,3-d]pyrimidin-4-one,
2-(endo-3-amino-3-methyl-8-azabicyclo[3.2.1]octan-8-yl)-5-(4-chloro-2-ethyl-2H-indazol-5-yl)-3-methyl-3,7-dihydro-4H-pyrrolo[2,3-d]pyrimidin-4-one,
2-(endo-3-amino-3-methyl-8-azabicyclo[3.2.1]octan-8-yl)-5-(4-chloro-2-methyl-2H-indazol-5-yl)-3-methyl-3,7-dihydro-4H-pyrrolo[2,3-d]pyrimidin-4-one,
2-(3-oxa-7,9-diazabicyclo[3.3.1]nonan-7-yl)-5-(4-chloro-2-methyl-2H-indazol-5-yl)-3-methyl-3,7-dihydro-4H-pyrrolo[2,3-d]pyrimidin-4-one,
(R)-5-(4-chloro-2-methyl-2H-indazol-5-yl)-3-methyl-2-(3-methylpiperazin-1-yl)-3,7-dihydro-4H-pyrrolo[2,3-d]pyrimidin-4-one,
(S)-5-(4-chloro-2-methyl-2H-indazol-5-yl)-2-(3-(hydroxymethyl)piperazin-1-yl)-3-methyl-3,7-dihydro-4H-pyrrolo[2,3-d]pyrimidin-4-one,
(R)-5-(4-chloro-2-methyl-2H-indazol-5-yl)-2-(3-(2-hydroxyethyl)piperazin-1-yl)-3-methyl-3,7-dihydro-4H-pyrrolo[2,3-d]pyrimidin-4-one,
2-((1S,4S)-2,5-diazabicyclo[2.2.1]heptan-2-yl)-5-(4-chloro-2-methyl-2H-indazol-5-yl)-3-methyl-3,7-dihydro-4H-pyrrolo[2,3-d]pyrimidin-4-one,
2-(7-amino-3-oxa-9-azabicyclo[3.3.1]nonan-9-yl)-5-(4-chloro-2-methyl-2H-indazol-5-yl)-3-methyl-3,7-dihydro-4H-pyrrolo[2,3-d]pyrimidin-4-one,
2-((1R,4R)-2,5-diazabicyclo[2.2.1]heptan-2-yl)-5-(4-chloro-2-methyl-2H-indazol-5-yl)-3-methyl-3,7-dihydro-4H-pyrrolo[2,3-d]pyrimidin-4-one,
2-(exo-8-amino-3-azabicyclo[3.2.1]octan-3-yl)-5-(4-chloro-2-methyl-2H-indazol-5-yl)-3-methyl-3,7-dihydro-4H-pyrrolo[2,3-d]pyrimidin-4-one,
2-(endo-8-amino-3-azabicyclo[3.2.1]octan-3-yl)-5-(4-chloro-2-methyl-2H-indazol-5-yl)-3-methyl-3,7-dihydro-4H-pyrrolo[2,3-d]pyrimidin-4-one,
rac-2-((1S,2R,3R,5R)-3-amino-2-fluoro-8-azabicyclo[3.2.1]octan-8-yl)-5-(4-chloro-2-methyl-2H-indazol-5-yl)-3-methyl-3,7-dihydro-4H-pyrrolo[2,3-d]pyrimidin-4-one,
2-(3,9-diazabicyclo[3.3.1]nonan-9-yl)-5-(4-chloro-2-methyl-2H-indazol-5-yl)-3-methyl-3,7-dihydro-4H-pyrrolo[2,3-d]pyrimidin-4-one,
2-(3,8-diazabicyclo[3.2.1]octan-3-yl)-5-(4-chloro-2-methyl-2H-indazol-5-yl)-3-methyl-3,7-dihydro-4H-pyrrolo[2,3-d]pyrimidin-4-one,
2-(3,6-diazabicyclo[3.1.1]heptan-6-yl)-5-(4-chloro-2-methyl-2H-indazol-5-yl)-3-methyl-3,7-dihydro-4H-pyrrolo[2,3-d]pyrimidin-4-one,
2-(3,9-diazabicyclo[3.3.1]nonan-3-yl)-5-(4-chloro-2-methyl-2H-indazol-5-yl)-3-methyl-3,7-dihydro-4H-pyrrolo[2,3-d]pyrimidin-4-one,
2-(3,6-diazabicyclo[3.1.1]heptan-3-yl)-5-(4-chloro-2-methyl-2H-indazol-5-yl)-3-methyl-3,7-dihydro-4H-pyrrolo[2,3-d]pyrimidin-4-one,
2-(2,5-diazabicyclo[2.2.2]octan-2-yl)-5-(4-chloro-2-methyl-2H-indazol-5-yl)-3-methyl-3,7-dihydro-4H-pyrrolo[2,3-d]pyrimidin-4-one,
2-(exo-6-amino-3-azabicyclo[3.1.1]heptan-3-yl)-5-(4-chloro-2-methyl-2H-indazol-5-yl)-3-methyl-3,7-dihydro-4H-pyrrolo[2,3-d]pyrimidin-4-one,
2-(endo-6-amino-3-azabicyclo[3.1.1]heptan-3-yl)-5-(4-chloro-2-methyl-2H-indazol-5-yl)-3-methyl-3,7-dihydro-4H-pyrrolo[2,3-d]pyrimidin-4-one,
2-(5-amino-2-azabicyclo[2.2.1]heptan-2-yl)-5-(4-chloro-2-methyl-2H-indazol-5-yl)-3-methyl-3,7-dihydro-4H-pyrrolo[2,3-d]pyrimidin-4-one,
2-(exo-3-amino-9-azabicyclo[3.3.1]nonan-9-yl)-5-(4-chloro-2-methyl-2H-indazol-5-yl)-3-methyl-3,7-dihydro-4H-pyrrolo[2,3-d]pyrimidin-4-one,
2-(endo-3-amino-9-azabicyclo[3.3.1]nonan-9-yl)-5-(4-chloro-2-methyl-2H-indazol-5-yl)-3-methyl-3,7-dihydro-4H-pyrrolo[2,3-d]pyrimidin-4-one,
5-(4-chloro-2-methyl-2H-indazol-5-yl)-3-methyl-2-(1,8-diazaspiro[4.5]decan-8-yl)-3,7-dihydro-4H-pyrrolo[2,3-d]pyrimidin-4-one,
2-(3,8-diazabicyclo[3.2.1]octan-8-yl)-5-(4-chloro-2-methyl-2H-indazol-5-yl)-3-methyl-3,7-dihydro-4H-pyrrolo[2,3-d]pyrimidin-4-one,
2-(3,7-diazabicyclo[4.2.0]octan-3-yl)-5-(4-chloro-2-methyl-2H-indazol-5-yl)-3-methyl-3,7-dihydro-4H-pyrrolo[2,3-d]pyrimidin-4-one,
5-(4-chloro-2-methyl-2H-indazol-5-yl)-3-methyl-2-(1,9-diazaspiro[5.5]undecan-9-yl)-3,7-dihydro-4H-pyrrolo[2,3-d]pyrimidin-4-one,
5-(4-chloro-2-methyl-2H-indazol-5-yl)-3-methyl-2-(1,7-diazaspiro[3.5]nonan-7-yl)-3,7-dihydro-4H-pyrrolo[2,3-d]pyrimidin-4-one,
(S)-2-(3-aminopyrrolidin-1-yl)-5-(4-chloro-2-methyl-2H-indazol-5-yl)-3-methyl-3,7-dihydro-4H-pyrrolo[2,3-d]pyrimidin-4-one,
(R)-2-(3-aminopyrrolidin-1-yl)-5-(4-chloro-2-methyl-2H-indazol-5-yl)-3-methyl-3,7-dihydro-4H-pyrrolo[2,3-d]pyrimidin-4-one,
(S)-5-(4-chloro-2-methyl-2H-indazol-5-yl)-3-methyl-2-(3-methylpiperazin-1-yl)-3,7-dihydro-4H-pyrrolo[2,3-d]pyrimidin-4-one,
2-((1S,2S,4R)-2-amino-7-azabicyclo[2.2.1]heptan-7-yl)-5-(4-chloro-2-methyl-2H-indazol-5-yl)-3-methyl-3,7-dihydro-4H-pyrrolo[2,3-d]pyrimidin-4-one,
(R)-5-(4-chloro-2-methyl-2H-indazol-5-yl)-3-methyl-2-(2-methylpiperazin-1-yl)-3,7-dihydro-4H-pyrrolo[2,3-d]pyrimidin-4-one,
(S)-5-(4-chloro-2-methyl-2H-indazol-5-yl)-3-methyl-2-(2-methylpiperazin-1-yl)-3,7-dihydro-4H-pyrrolo[2,3-d]pyrimidin-4-one,
2-((1R,2R,4S)-2-amino-7-azabicyclo[2.2.1]heptan-7-yl)-5-(4-chloro-2-methyl-2H-indazol-5-yl)-3-methyl-3,7-dihydro-4H-pyrrolo[2,3-d]pyrimidin-4-one,
2-((3R,4S)-3-amino-4-fluoropyrrolidin-1-yl)-5-(4-chloro-2-methyl-2H-indazol-5-yl)-3-methyl-3,7-dihydro-4H-pyrrolo[2,3-d]pyrimidin-4-one,
rac-2-((1S,4S,7S)-7-amino-2-azabicyclo[2.2.1]heptan-2-yl)-5-(4-chloro-2-methyl-2H-indazol-5-yl)-3-methyl-3,7-dihydro-4H-pyrrolo[2,3-d]pyrimidin-4-one,
2-((3S,4S)-3-amino-4-fluoropyrrolidin-1-yl)-5-(4-chloro-2-methyl-2H-indazol-5-yl)-3-methyl-3,7-dihydro-4H-pyrrolo[2,3-d]pyrimidin-4-one,
2-(4-amino-3,3-difluoropyrrolidin-1-yl)-5-(4-chloro-2-methyl-2H-indazol-5-yl)-3-methyl-3,7-dihydro-4H-pyrrolo[2,3-d]pyrimidin-4-one,
(S)-2-(3-amino-3-methylpyrrolidin-1-yl)-5-(4-chloro-2-methyl-2H-indazol-5-yl)-3-methyl-3,7-dihydro-4H-pyrrolo[2,3-d]pyrimidin-4-one,
(R)-2-(3-amino-3-methylpyrrolidin-1-yl)-5-(4-chloro-2-methyl-2H-indazol-5-yl)-3-methyl-3,7-dihydro-4H-pyrrolo[2,3-d]pyrimidin-4-one,
2-((3R,4R)-3-amino-4-methylpyrrolidin-1-yl)-5-(4-chloro-2-methyl-2H-indazol-5-yl)-3-methyl-3,7-dihydro-4H-pyrrolo[2,3-d]pyrimidin-4-one,
2-((3R,4S)-3-amino-4-methylpyrrolidin-1-yl)-5-(4-chloro-2-methyl-2H-indazol-5-yl)-3-methyl-3,7-dihydro-4H-pyrrolo[2,3-d]pyrimidin-4-one,
2-(3-amino-3-(hydroxymethyl)pyrrolidin-1-yl)-5-(4-chloro-2-methyl-2H-indazol-5-yl)-3-methyl-3,7-dihydro-4H-pyrrolo[2,3-d]pyrimidin-4-one,
2-((3S,4S)-4-amino-3-methyl-2-oxa-8-azaspiro[4.5]decan-8-yl)-5-(4-chloro-2-methyl-2H-indazol-5-yl)-3-methyl-3,7-dihydro-4H-pyrrolo[2,3-d]pyrimidin-4-one,
(R)-2-(3-(aminomethyl)pyrrolidin-1-yl)-5-(4-chloro-2-methyl-2H-indazol-5-yl)-3-methyl-3,7-dihydro-4H-pyrrolo[2,3-d]pyrimidin-4-one,
2-(1-amino-3-azabicyclo[3.1.0]hexan-3-yl)-5-(4-chloro-2-methyl-2H-indazol-5-yl)-3-methyl-3,7-dihydro-4H-pyrrolo[2,3-d]pyrimidin-4-one,
2-(6-amino-3-azabicyclo[3.1.0]hexan-3-yl)-5-(4-chloro-2-methyl-2H-indazol-5-yl)-3-methyl-3,7-dihydro-4H-pyrrolo[2,3-d]pyrimidin-4-one,
(S)-2-(3-(aminomethyl)pyrrolidin-1-yl)-5-(4-chloro-2-methyl-2H-indazol-5-yl)-3-methyl-3,7-dihydro-4H-pyrrolo[2,3-d]pyrimidin-4-one,
2-(4-(aminomethyl)-4-methoxypiperidin-1-yl)-5-(4-chloro-2-methyl-2H-indazol-5-yl)-3-methyl-3,7-dihydro-4H-pyrrolo[2,3-d]pyrimidin-4-one,
2-(4-(aminomethyl)-4-fluoropiperidin-1-yl)-5-(4-chloro-2-methyl-2H-indazol-5-yl)-3-methyl-3,7-dihydro-4H-pyrrolo[2,3-d]pyrimidin-4-one,
2-(3,9-diazabicyclo[3.3.1]nonan-9-yl)-5-(4-chloro-2-ethyl-2H-indazol-5-yl)-3-methyl-3,7-dihydro-4H-pyrrolo[2,3-d]pyrimidin-4-one,
2-(endo-3-amino-8-azabicyclo[3.2.1]octan-8-yl)-3-methyl-5-(2-methyl-2H-indazol-5-yl)-3,7-dihydro-4H-pyrrolo[2,3-d]pyrimidin-4-one,
2-(endo-3-amino-8-azabicyclo[3.2.1]octan-8-yl)-5-(7-chloro-2-methylbenzo[d]oxazol-6-yl)-3-methyl-3,7-dihydro-4H-pyrrolo[2,3-d]pyrimidin-4-one,
2-(endo-3-amino-8-azabicyclo[3.2.1]octan-8-yl)-5-(7-chloro-2-ethylbenzo[d]oxazol-6-yl)-3-methyl-3,7-dihydro-4H-pyrrolo[2,3-d]pyrimidin-4-one,
2-(endo-3-amino-8-azabicyclo[3.2.1]octan-8-yl)-5-(4-fluoro-2-methyl-2H-indazol-5-yl)-3-methyl-3,7-dihydro-4H-pyrrolo[2,3-d]pyrimidin-4-one,
2-(endo-3-amino-8-azabicyclo[3.2.1]octan-8-yl)-5-(6,7-difluoro-1-methyl-1H-benzo[d][1,2,3]triazol-5-yl)-3-methyl-3,7-dihydro-4H-pyrrolo[2,3-d]pyrimidin-4-one,
2-(endo-3-amino-8-azabicyclo[3.2.1]octan-8-yl)-5-(6-fluoro-2-methyl-2H-indazol-5-yl)-3-methyl-3,7-dihydro-4H-pyrrolo[2,3-d]pyrimidin-4-one,
2-(endo-3-amino-8-azabicyclo[3.2.1]octan-8-yl)-5-(4-methoxy-2-methyl-2H-indazol-5-yl)-3-methyl-3,7-dihydro-4H-pyrrolo[2,3-d]pyrimidin-4-one,
2-(endo-3-amino-8-azabicyclo[3.2.1]octan-8-yl)-5-(7-chloro-1-methyl-1H-benzo[d][1,2,3]triazol-6-yl)-3-methyl-3,7-dihydro-4H-pyrrolo[2,3-d]pyrimidin-4-one,
2-(endo-3-amino-8-azabicyclo[3.2.1]octan-8-yl)-5-(4-chloro-2-(2-hydroxy-2-methylpropyl)-2H-indazol-5-yl)-3-methyl-3,7-dihydro-4H-pyrrolo[2,3-d]pyrimidin-4-one,
2-(endo-3-amino-8-azabicyclo[3.2.1]octan-8-yl)-5-(4-chloro-2,7-dimethyl-2H-indazol-5-yl)-3-methyl-3,7-dihydro-4H-pyrrolo[2,3-d]pyrimidin-4-one,
2-(endo-3-amino-8-azabicyclo[3.2.1]octan-8-yl)-5-(1H-indazol-5-yl)-3-methyl-3,7-dihydro-4H-pyrrolo[2,3-d]pyrimidin-4-one,
2-(endo-3-amino-8-azabicyclo[3.2.1]octan-8-yl)-5-(3,4-dichloro-2-methyl-2H-indazol-5-yl)-3-methyl-3,7-dihydro-4H-pyrrolo[2,3-d]pyrimidin-4-one,
2-(endo-3-amino-3-methyl-8-azabicyclo[3.2.1]octan-8-yl)-5-(3,4-dichloro-2-methyl-2H-indazol-5-yl)-3-methyl-3,7-dihydro-4H-pyrrolo[2,3-d]pyrimidin-4-one,
2-(3-oxa-7,9-diazabicyclo[3.3.1]nonan-7-yl)-5-(3,4-dichloro-2-methyl-2H-indazol-5-yl)-3-methyl-3,7-dihydro-4H-pyrrolo[2,3-d]pyrimidin-4-one,
2-(3-oxa-7,9-diazabicyclo[3.3.1]nonan-7-yl)-5-(7-chloro-2-methylbenzo[d]thiazol-6-yl)-3-methyl-3,7-dihydro-4H-pyrrolo[2,3-d]pyrimidin-4-one,
2-(5-(2-(3,9-diazabicyclo[3.3.1]nonan-9-yl)-3-methyl-4-oxo-4,7-dihydro-3H-pyrrolo[2,3-d]pyrimidin-5-yl)-3,4-dichloro-2H-indazol-2-yl)-N,N-dimethylacetamide,
3-(5-(2-(3,9-diazabicyclo[3.3.1]nonan-9-yl)-3-methyl-4-oxo-4,7-dihydro-3H-pyrrolo[2,3-d]pyrimidin-5-yl)-3,4-dichloro-2H-indazol-2-yl)-N,N-dimethylpropanamide,
2-(6-(2-(3,9-diazabicyclo[3.3.1]nonan-9-yl)-3-methyl-4-oxo-4,7-dihydro-3H-pyrrolo[2,3-d]pyrimidin-5-yl)-7-chlorobenzo[d]thiazol-2-yl)-N,N-dimethylacetamide,
2-(3-oxa-7,9-diazabicyclo[3.3.1]nonan-9-yl)-5-(4-chloro-2-methyl-2H-indazol-5-yl)-3-methyl-3,7-dihydro-4H-pyrrolo[2,3-d]pyrimidin-4-one,
2-(5-(2-(3,8-diazabicyclo[3.2.1]octan-8-yl)-3-methyl-4-oxo-4,7-dihydro-3H-pyrrolo[2,3-d]pyrimidin-5-yl)-4-chloro-2H-indazol-2-yl)-N,N-dimethylacetamide,
2-(3,8-diazabicyclo[3.2.1]octan-8-yl)-5-(4-chloro-2-methyl-2H-benzo[d][1,2,3]triazol-5-yl)-3-methyl-3,7-dihydro-4H-pyrrolo[2,3-d]pyrimidin-4-one,
3-(5-(2-(3,8-diazabicyclo[3.2.1]octan-8-yl)-3-methyl-4-oxo-4,7-dihydro-3H-pyrrolo[2,3-d]pyrimidin-5-yl)-4-chloro-2H-indazol-2-yl)-N,N-dimethylpropanamide,
3-(5-(2-(3,8-diazabicyclo[3.2.1]octan-8-yl)-3-methyl-4-oxo-4,7-dihydro-3H-pyrrolo[2,3-d]pyrimidin-5-yl)-3,4-dichloro-2H-indazol-2-yl)-N,N-dimethylpropanamide,
2-(3,8-diazabicyclo[3.2.1]octan-8-yl)-5-(7-chloro-2-methylbenzo[d]thiazol-6-yl)-3-methyl-3,7-dihydro-4H-pyrrolo[2,3-d]pyrimidin-4-one,
2-(3,8-diazabicyclo[3.2.1]octan-8-yl)-5-(4-chloro-2-ethyl-2H-indazol-5-yl)-3-methyl-3,7-dihydro-4H-pyrrolo[2,3-d]pyrimidin-4-one,
2-((1R,2R,4S)-2-amino-7-azabicyclo[2.2.1]heptan-7-yl)-5-(4-chloro-2-ethyl-2H-indazol-5-yl)-3-methyl-3,7-dihydro-4H-pyrrolo[2,3-d]pyrimidin-4-one,
2-((1R,2R,4S)-2-amino-7-azabicyclo[2.2.1]heptan-7-yl)-5-(3,4-dichloro-2-methyl-2H-indazol-5-yl)-3-methyl-3,7-dihydro-4H-pyrrolo[2,3-d]pyrimidin-4-one,
2-((1R,2R,4S)-2-amino-7-azabicyclo[2.2.1]heptan-7-yl)-5-(4-chloro-2,3-dimethyl-2H-indazol-5-yl)-3-methyl-3,7-dihydro-4H-pyrrolo[2,3-d]pyrimidin-4-one,
2-((1R,2R,4S)-2-amino-7-azabicyclo[2.2.1]heptan-7-yl)-5-(7-chloro-2-methylbenzo[d]thiazol-6-yl)-3-methyl-3,7-dihydro-4H-pyrrolo[2,3-d]pyrimidin-4-one,
2-((1R,2R,4S)-2-amino-7-azabicyclo[2.2.1]heptan-7-yl)-5-(3-chloro-4-fluoro-2-methyl-2H-indazol-5-yl)-3-methyl-3,7-dihydro-4H-pyrrolo[2,3-d]pyrimidin-4-one,
5-(4-chloro-2-methyl-2H-indazol-5-yl)-3-methyl-2-((1R,2R,4S)-2-(methylamino)-7-azabicyclo[2.2.1]heptan-7-yl)-3,7-dihydro-4H-pyrrolo[2,3-d]pyrimidin-4-one,
5-(4-chloro-2-ethyl-2H-indazol-5-yl)-3-methyl-2-((1R,2R,4S)-2-(methylamino)-7-azabicyclo[2.2.1]heptan-7-yl)-3,7-dihydro-4H-pyrrolo[2,3-d]pyrimidin-4-one,
2-(3,9-diazabicyclo[3.3.1]nonan-9-yl)-5-(3,4-dichloro-2-methyl-2H-indazol-5-yl)-3-methyl-3,7-dihydro-4H-pyrrolo[2,3-d]pyrimidin-4-one,
2-(3,9-diazabicyclo[3.3.1]nonan-9-yl)-5-(3-chloro-4-fluoro-2-methyl-2H-indazol-5-yl)-3-methyl-3,7-dihydro-4H-pyrrolo[2,3-d]pyrimidin-4-one,
2-((1R,2R,4S)-2-amino-7-azabicyclo[2.2.1]heptan-7-yl)-5-(7-chlorobenzo[d]thiazol-6-yl)-3-methyl-3,7-dihydro-4H-pyrrolo[2,3-d]pyrimidin-4-one,
2-((1R,2R,4S)-2-amino-7-azabicyclo[2.2.1]heptan-7-yl)-5-(4-chloro-2-ethyl-3-methoxy-2H-indazol-5-yl)-3-methyl-3,7-dihydro-4H-pyrrolo[2,3-d]pyrimidin-4-one,
2-((1R,2R,4S)-2-amino-7-azabicyclo[2.2.1]heptan-7-yl)-5-(3,4-dichloro-2-(fluoromethyl)-2H-indazol-5-yl)-3-methyl-3,7-dihydro-4H-pyrrolo[2,3-d]pyrimidin-4-one,
5-(3,4-dichloro-2-methyl-2H-indazol-5-yl)-3-methyl-2-((1R,2R,4S)-2-(methylamino)-7-azabicyclo[2.2.1]heptan-7-yl)-3,7-dihydro-4H-pyrrolo[2,3-d]pyrimidin-4-one,
5-(7-chlorobenzo[d]thiazol-6-yl)-3-methyl-2-((1R,2R,4S)-2-(methylamino)-7-azabicyclo[2.2.1]heptan-7-yl)-3,7-dihydro-4H-pyrrolo[2,3-d]pyrimidin-4-one,
5-(3-chloro-4-fluoro-2-methyl-2H-indazol-5-yl)-3-methyl-2-((1R,2R,4S)-2-(methylamino)-7-azabicyclo[2.2.1]heptan-7-yl)-3,7-dihydro-4H-pyrrolo[2,3-d]pyrimidin-4-one,
5-(7-chloro-2-methylbenzo[d]thiazol-6-yl)-3-methyl-2-((1R,2R,4S)-2-(methylamino)-7-azabicyclo[2.2.1]heptan-7-yl)-3,7-dihydro-4H-pyrrolo[2,3-d]pyrimidin-4-one,
2-(3,9-diazabicyclo[3.3.1]nonan-9-yl)-5-(7-chlorobenzo[d]thiazol-6-yl)-3-methyl-3,7-dihydro-4H-pyrrolo[2,3-d]pyrimidin-4-one,
2-((1R,2R,4S)-2-amino-7-azabicyclo[2.2.1]heptan-7-yl)-5-(4-chloro-3-(difluoromethyl)-2-ethyl-2H-indazol-5-yl)-3-methyl-3,7-dihydro-4H-pyrrolo[2,3-d]pyrimidin-4-one,
2-((1R,2R,4S)-2-amino-7-azabicyclo[2.2.1]heptan-7-yl)-5-(4-chloro-2-ethyl-3-(hydroxymethyl)-2H-indazol-5-yl)-3-methyl-3,7-dihydro-4H-pyrrolo[2,3-d]pyrimidin-4-one,
6-(3,9-diazabicyclo[3.3.1]nonan-9-yl)-3-(4-chloro-2-ethyl-2H-indazol-5-yl)-5-methyl-1,5-dihydro-4H-pyrazolo[3,4-d]pyrimidin-4-one,
6-((1R,2R,4S)-2-amino-7-azabicyclo[2.2.1]heptan-7-yl)-3-(4-chloro-2-ethyl-2H-indazol-5-yl)-5-methyl-1,5-dihydro-4H-pyrazolo[3,4-d]pyrimidin-4-one,
6-((1R,2R,4S)-2-amino-7-azabicyclo[2.2.1]heptan-7-yl)-3-(7-chloro-2-methylbenzo[d]thiazol-6-yl)-5-methyl-1,5-dihydro-4H-pyrazolo[3,4-d]pyrimidin-4-one,
6-((1R,2R,4S)-2-amino-7-azabicyclo[2.2.1]heptan-7-yl)-3-(3,4-dichloro-2-methyl-2H-indazol-5-yl)-5-methyl-1,5-dihydro-4H-pyrazolo[3,4-d]pyrimidin-4-one,
6-(endo-3-amino-3-methyl-8-azabicyclo[3.2.1]octan-8-yl)-3-(4-chloro-2-ethyl-2H-indazol-5-yl)-5-methyl-1,5-dihydro-4H-pyrazolo[3,4-d]pyrimidin-4-one,
6-(3,9-diazabicyclo[3.3.1]nonan-9-yl)-3-(3,4-dichloro-2-methyl-2H-indazol-5-yl)-5-methyl-1,5-dihydro-4H-pyrazolo[3,4-d]pyrimidin-4-one,
6-(3,9-diazabicyclo[3.3.1]nonan-9-yl)-3-(4-chloro-2-methyl-2H-indazol-5-yl)-5-methyl-1,5-dihydro-4H-pyrazolo[3,4-d]pyrimidin-4-one,
3-(4-chloro-2-ethyl-2H-indazol-5-yl)-5-methyl-6-((1R,2R,4S)-2-(methylamino)-7-azabicyclo[2.2.1]heptan-7-yl)-1,5-dihydro-4H-pyrazolo[3,4-d]pyrimidin-4-one,
3-(3,4-dichloro-2-methyl-2H-indazol-5-yl)-5-methyl-6-((1R,2R,4S)-2-(methylamino)-7-azabicyclo[2.2.1]heptan-7-yl)-1,5-dihydro-4H-pyrazolo[3,4-d]pyrimidin-4-one,
3-(4-chloro-2-methyl-2H-indazol-5-yl)-5-methyl-6-((1R,2R,4S)-2-(methylamino)-7-azabicyclo[2.2.1]heptan-7-yl)-1,5-dihydro-4H-pyrazolo[3,4-d]pyrimidin-4-one,
3-(3-chloro-4-fluoro-2-methyl-2H-indazol-5-yl)-5-methyl-6-((1R,2R,4S)-2-(methylamino)-7-azabicyclo[2.2.1]heptan-7-yl)-1,5-dihydro-4H-pyrazolo[3,4-d]pyrimidin-4-one,
6-(3,9-diazabicyclo[3.3.1]nonan-9-yl)-3-(7-chlorobenzo[d]thiazol-6-yl)-5-methyl-1,5-dihydro-4H-pyrazolo[3,4-d]pyrimidin-4-one,
3-(7-chlorobenzo[d]thiazol-6-yl)-5-methyl-6-((1R,2R,4S)-2-(methylamino)-7-azabicyclo[2.2.1]heptan-7-yl)-1,5-dihydro-4H-pyrazolo[3,4-d]pyrimidin-4-one,
3-(7-chloro-2-methylbenzo[d]thiazol-6-yl)-5-methyl-6-((1R,2R,4S)-2-(methylamino)-7-azabicyclo[2.2.1]heptan-7-yl)-1,5-dihydro-4H-pyrazolo[3,4-d]pyrimidin-4-one,
rac-6-((1S,4S,7S)-7-amino-2-azabicyclo[2.2.1]heptan-2-yl)-3-(4-chloro-2-ethyl-2H-indazol-5-yl)-5-methyl-1,5-dihydro-4H-pyrazolo[3,4-d]pyrimidin-4-one,
rac-6-((1S,4S,7S)-7-amino-2-azabicyclo[2.2.1]heptan-2-yl)-3-(3,4-dichloro-2-methyl-2H-indazol-5-yl)-5-methyl-1,5-dihydro-4H-pyrazolo[3,4-d]pyrimidin-4-one,
6-((1R,2R,4S)-2-amino-7-azabicyclo[2.2.1]heptan-7-yl)-3-(3,4-dichloro-2-ethyl-2H-indazol-5-yl)-5-methyl-1,5-dihydro-4H-pyrazolo[3,4-d]pyrimidin-4-one,
3-(3,4-dichloro-2-ethyl-2H-indazol-5-yl)-5-methyl-6-((1R,2R,4S)-2-(methylamino)-7-azabicyclo[2.2.1]heptan-7-yl)-1,5-dihydro-4H-pyrazolo[3,4-d]pyrimidin-4-one,
2-((1R,2S,3R,5S)-3-amino-2-fluoro-8-azabicyclo[3.2.1]octan-8-yl)-5-(4-chloro-2-methyl-2H-indazol-5-yl)-3-methyl-3,7-dihydro-4H-pyrrolo[2,3-d]pyrimidin-4-one,
2-((1S,2R,3S,5R)-3-amino-2-fluoro-8-azabicyclo[3.2.1]octan-8-yl)-5-(4-chloro-2-methyl-2H-indazol-5-yl)-3-methyl-3,7-dihydro-4H-pyrrolo[2,3-d]pyrimidin-4-one,
2-((1R,2R,3R,5S)-3-amino-2-fluoro-8-azabicyclo[3.2.1]octan-8-yl)-5-(4-chloro-2-methyl-2H-indazol-5-yl)-3-methyl-3,7-dihydro-4H-pyrrolo[2,3-d]pyrimidin-4-one,
2-((1S,2S,3S,5R)-3-amino-2-fluoro-8-azabicyclo[3.2.1]octan-8-yl)-5-(4-chloro-2-methyl-2H-indazol-5-yl)-3-methyl-3,7-dihydro-4H-pyrrolo[2,3-d]pyrimidin-4-one,
rac-2-((1S,4S,7S)-7-amino-2-azabicyclo[2.2.1]heptan-2-yl)-5-(4-chloro-2-ethyl-2H-indazol-5-yl)-3-methyl-3,7-dihydro-4H-pyrrolo[2,3-d]pyrimidin-4-one,
rac-((1S,4S,7S)-7-amino-2-azabicyclo[2.2.1]heptan-2-yl)-3-(7-chlorobenzo[d]thiazol-6-yl)-5-methyl-1,5-dihydro-4H-pyrazolo[3,4-d]pyrimidin-4-one,
rac-5-(4-chloro-2-methyl-2H-indazol-5-yl)-3-methyl-2-((1S,4S,7S)-7-(methylamino)-2-azabicyclo[2.2.1]heptan-2-yl)-3,7-dihydro-4H-pyrrolo[2,3-d]pyrimidin-4-one,
endo-6-[3-amino-8-azabicyclo[3.2.1]octan-8-yl]-3-(4-chloro-2-methyl-2H-indazol-5-yl)-5-methyl-1H,4H,5H-pyrazolo[3,4-d]pyrimidin-4-one,
3-(4-chloro-2-methyl-2H-indazol-5-yl)-6-{3,8-diazabicyclo[3.2.1]octan-8-yl}-5-methyl-1H,4H,5H-pyrazolo[3,4-d]pyrimidin-4-one,
6-[(1R,3S)-1-amino-3-hydroxy-8-azaspiro[4.5]decan-8-yl]-3-(4-chloro-2-methyl-2H-indazol-5-yl)-5-methyl-1H,4H,5H-pyrazolo[3,4-d]pyrimidin-4-one,
6-[(4S)-4-amino-2-oxa-8-azaspiro[4.5]decan-8-yl]-3-(4-chloro-2-methyl-2H-indazol-5-yl)-5-methyl-1H,4H,5H-pyrazolo[3,4-d]pyrimidin-4-one,
exo-6-[3-amino-8-azabicyclo[3.2.1]octan-8-yl]-3-(4-chloro-2-methyl-2H-indazol-5-yl)-5-methyl-1H,4H,5H-pyrazolo[3,4-d]pyrimidin-4-one,
3-(4-chloro-2-methyl-2H-indazol-5-yl)-6-{2,7-diazaspiro[3.5]nonan-7-yl}-5-methyl-1H,4H,5H-pyrazolo[3,4-d]pyrimidin-4-one,
6-[(1R)-1-amino-8-azaspiro[4.5]decan-8-yl]-3-(4-chloro-2-methyl-2H-indazol-5-yl)-5-methyl-1H,4H,5H-pyrazolo[3,4-d]pyrimidin-4-one,
6-[(3S,4S)-4-amino-3-methyl-2-oxa-8-azaspiro[4.5]decan-8-yl]-3-(4-chloro-2-methyl-2H-indazol-5-yl)-5-methyl-1H,4H,5H-pyrazolo[3,4-d]pyrimidin-4-one,
6-[(1R,3R)-1-amino-3-fluoro-8-azaspiro[4.5]decan-8-yl]-3-(4-chloro-2-methyl-2H-indazol-5-yl)-5-methyl-1H,4H,5H-pyrazolo[3,4-d]pyrimidin-4-one,
3-(4-chloro-2-methyl-2H-indazol-5-yl)-5-methyl-6-{3-oxa-7,9-diazabicyclo[3.3.1]nonan-7-yl}-1H,4H,5H-pyrazolo[3,4-d]pyrimidin-4-one,
3-(3,4-dichloro-2-methyl-2H-indazol-5-yl)-5-methyl-6-{3-oxa-7,9-diazabicyclo[3.3.1]nonan-7-yl}-1H,4H,5H-pyrazolo[3,4-d]pyrimidin-4-one,
6-[(3S,4S)-4-amino-3-methyl-2-oxa-8-azaspiro[4.5]decan-8-yl]-3-(3,4-dichloro-2-methyl-2H-indazol-5-yl)-5-methyl-1H,4H,5H-pyrazolo[3,4-d]pyrimidin-4-one,
6-[(3S,4S)-4-amino-3-methyl-2-oxa-8-azaspiro[4.5]decan-8-yl]-3-(4-chloro-2-ethyl-2H-indazol-5-yl)-5-methyl-1H,4H,5H-pyrazolo[3,4-d]pyrimidin-4-one,
6-[(1S,2R,3S,5R)-3-amino-2-fluoro-8-azabicyclo[3.2.1]octan-8-yl]-3-(4-chloro-2-methyl-2H-indazol-5-yl)-5-methyl-1H,4H,5H-pyrazolo[3,4-d]pyrimidin-4-one,
2-[(3S,4S)-4-amino-3-methyl-2-oxa-8-azaspiro[4.5]decan-8-yl]-5-(4-chloro-2-ethyl-2H-indazol-5-yl)-3-methyl-3H,4H,7H-pyrrolo[2,3-d]pyrimidin-4-one,
5-(4-chloro-2-ethyl-2H-indazol-5-yl)-2-(1,4-diazepan-1-yl)-3-methyl-3H,4H,7H-pyrrolo[2,3-d]pyrimidin-4-one,
rac-2-[(1R,2R,5R)-2-amino-8-azabicyclo[3.2.1]octan-8-yl]-5-(4-chloro-2-methyl-2H-indazol-5-yl)-3-methyl-3H,4H,7H-pyrrolo[2,3-d]pyrimidin-4-one,
rac-5-(4-chloro-2-methyl-2H-indazol-5-yl)-2-[(1R,6S)-3,9-diazabicyclo[4.2.1]nonan-9-yl]-3-methyl-3H,4H,7H-pyrrolo[2,3-d]pyrimidin-4-one,
rac-2-(4-aminoazepan-1-yl)-5-(4-chloro-2-ethyl-2H-indazol-5-yl)-3-methyl-3H,4H,7H-pyrrolo[2,3-d]pyrimidin-4-one,
rel-2-((1R,4R,7R)-7-amino-2-azabicyclo[2.2.1]heptan-2-yl)-5-(4-chloro-2-methyl-2H-indazol-5-yl)-3-methyl-3,7-dihydro-4H-pyrrolo[2,3-d]pyrimidin-4-one,
rel-2-((1S,4S,7S)-7-amino-2-azabicyclo[2.2.1]heptan-2-yl)-5-(4-chloro-2-methyl-2H-indazol-5-yl)-3-methyl-3,7-dihydro-4H-pyrrolo[2,3-d]pyrimidin-4-one,
rel-2-((1R,4R,7R)-7-amino-2-azabicyclo[2.2.1]heptan-2-yl)-5-(4-chloro-2-methyl-2H-indazol-5-yl)-3-methyl-3,7-dihydro-4H-pyrrolo[2,3-d]pyrimidin-4-one,
rel-2-((1S,4S,7S)-7-Amino-2-azabicyclo[2.2.1]heptan-2-yl)-5-(4-chloro-2-methyl-2H-indazol-5-yl)-3-methyl-3,7-dihydro-4H-pyrrolo[2,3-d]pyrimidin-4-one, and
5-(2-((1R,2R,4S)-2-amino-7-azabicyclo[2.2.1]heptan-7-yl)-3-methyl-4-oxo-4,7-dihydro-3H-pyrrolo[2,3-d]pyrimidin-5-yl)-4-chloro-2-ethyl-2H-indazole-3-carbonitrile.

The following are examples of more preferable compounds of the present invention:
2-((1R,2R,4S)-2-amino-7-azabicyclo[2.2.1]heptan-7-yl)-5-(3,4-dichloro-2-methyl-2H-indazol-5-yl)-3-methyl-3,7-dihydro-4H-pyrrolo[2,3-d]pyrimidin-4-one,6-((1R,4R,7R)-7-amino-2-azabicyclo[2.2.1]heptan-2-yl)-3-(3,4-dichloro-2-methyl-2H-indazol-5-yl)-5-methyl-1,5-dihydro-4H-pyrazolo[3,4-d]pyrimidin-4-one,
2-((1R,4R,7R)-7-amino-2-azabicyclo[2.2.1]heptan-2-yl)-5-(4-chloro-2-ethyl-2H-indazol-5-yl)-3-methyl-3,7-dihydro-4H-pyrrolo[2,3-d]pyrimidin-4-one,
2-((1R,4R,7R)-7-amino-2-azabicyclo[2.2.1]heptan-2-yl)-5-(4-chloro-2-methyl-2H-indazol-5-yl)-3-methyl-3,7-dihydro-4H-pyrrolo[2,3-d]pyrimidin-4-one,
2-((1R,4R,7R)-7-amino-2-azabicyclo[2.2.1]heptan-2-yl)-5-(3,4-dichloro-2-methyl-2H-indazol-5-yl)-3-methyl-3,7-dihydro-4H-pyrrolo[2,3-d]pyrimidin-4-one,
2-((1R,2R,4S)-2-amino-7-azabicyclo[2.2.1]heptan-7-yl)-5-(3-chloro-4-fluoro-2-methyl-2H-indazol-5-yl)-3-methyl-3,7-dihydro-4H-pyrrolo[2,3-d]pyrimidin-4-one, and
2-((1R,2R,4S)-2-amino-7-azabicyclo[2.2.1]heptan-7-yl)-5-(4-chloro-2-ethyl-2H-indazol-5-yl)-3-methyl-3,7-dihydro-4H-pyrrolo[2,3-d]pyrimidin-4-one.

The following are examples of even more preferable compounds of the present invention:

### 2-((1R,2R,4S)-2-amino-7-azabicyclo[2.2.1]heptan-7-yl)-5-(3,4-dichloro-2-methyl-2H-indazol-5-yl)-3-methyl-3,7-dihydro-4H-pyrrolo[2,3-d]pyrimidin-4-one

In the present specification, 2-((1R,2R,4S)-2-amino-7-azabicyclo[2.2.1]heptan-7-yl)-5-(3,4-dichloro-2-methyl-2H-indazol-5-yl)-3-methyl-3,7-dihydro-4H-pyrrolo[2,3-d]pyrimidin-4-one is referred to as “Compound 1” for the sake of convenience. Compound 1 can be prepared as shown in Preparation Example 1.

In the present specification, 6-((1R,4R,7R)-7-amino-2-azabicyclo[2.2.1]heptan-2-yl)-3-(3,4-dichloro-2-methyl-2H-indazol-5-yl)-5-methyl-1,5-dihydro-4H-pyrazolo[3,4-d]pyrimidin-4-one is referred to as “Compound 2” for the sake of convenience. Compound 2 can be prepared as shown in Synthetic Example shown as below.

In the present specification, 2-((1R,4R,7R)-7-amino-2-azabicyclo[2.2.1]heptan-2-yl)-5-(4-chloro-2-ethyl-2H-indazol-5-yl)-3-methyl-3,7-dihydro-4H-pyrrolo[2,3-d]pyrimidin-4-one is referred to as “Compound 3” for the sake of convenience. Compound 3 can be prepared as shown in Synthetic Example shown as below.

In the present specification, 2-((1R,4R,7R)-7-amino-2-azabicyclo[2.2.1]heptan-2-yl)-5-(4-chloro-2-methyl-2H-indazol-5-yl)-3-methyl-3,7-dihydro-4H-pyrrolo[2,3-d]pyrimidin-4-one is referred to as “Compound 4” for the sake of convenience. Compound 4 can be prepared as shown in Synthetic Example shown as below.

In the present specification, 2-((1R,4R,7R)-7-amino-2-azabicyclo[2.2.1]heptan-2-yl)-5-(3,4-dichloro-2-methyl-2H-indazol-5-yl)-3-methyl-3,7-dihydro-4H-pyrrolo[2,3-d]pyrimidin-4-one is referred to as “Compound 5” for the sake of convenience. Compound 5 can be prepared as shown in Synthetic Example shown as below.

In the present specification, 2-((1R,2R,4S)-2-amino-7-azabicyclo[2.2.1]heptan-7-yl)-5-(3-chloro-4-fluoro-2-methyl-2H-indazol-5-yl)-3-methyl-3,7-dihydro-4H-pyrrolo[2,3-d]pyrimidin-4-one is referred to as “Compound 6” for the sake of convenience. Compound 6 can be prepared as shown in Synthetic Example shown as below.

In the present specification, 2-((1R,2R,4S)-2-amino-7-azabicyclo[2.2.1]heptan-7-yl)-5-(4-chloro-2-ethyl-2H-indazol-5-yl)-3-methyl-3,7-dihydro-4H-pyrrolo[2,3-d]pyrimidin-4-one is referred to as “Compound 7” for the sake of convenience. Compound 7 can be prepared as shown in Synthetic Example shown as below.

In the present invention, the compound represented by formula (I) can be used directly or in the form of a pharmaceutically acceptable salt. The pharmaceutically acceptable salt of the compound represented by formula (I) is not particularly limited, and examples thereof include addition salts with inorganic acids such as hydrochloric acid, hydrobromic acid, perchloric acid, nitric acid, phosphoric acid and sulfuric acid, organic acids such as acetic acid, lactic acid, citric acid, oxalic acid, succinic acid, malic acid, tartaric acid, fumaric acid, maleic acid, methanesulfonic acid, benzenesulfonic acid, p-toluenesulfonic acid. In this context, the “additional compound having an antitumor effect or pharmaceutically acceptable salt thereof” is intended to exclude the compound represented by formula (I), because the compound represented by formula (I) is an antitumor agent based on an SHP2 inhibitory effect.

The compound represented by formula (I) or a pharmaceutically acceptable salt thereof is an antitumor agent that has an excellent SHP2 inhibitory effect and has reduced side effects. When concomitantly used with various additional compounds having an antitumor effect, the compound represented by formula (I) has an effect of enhancing the antitumor effects of the additional compounds having an antitumor effect without remarkably exacerbating toxicity.

Accordingly, the combination drug of the present invention comprises at least one additional compound having an antitumor effect, at least one tautomer thereof, at least one solvate thereof, or at least one pharmaceutically acceptable salt thereof, which is different from the compound represented by formula (I) or a pharmaceutically acceptable salt thereof. In the present specification, “the additional compound having an antitumor effect or at least one pharmaceutically acceptable salt thereof, selected from the group consisting of molecular targeted drugs and cytotoxic drugs” is referred to as “AC” for the sake of convenience. The combination drug preferably has one AC.

The molecular targeted drug includes a tyrosine kinase inhibitor, a RAS-MAPK pathway inhibitor, a PI3K pathway inhibitor, a BCL2 inhibitor, a CDK4/6 inhibitor, and an HDAC inhibitor.

Examples of tyrosine kinase inhibitor are ALK inhibitor, Her family inhibitors (EGFR and HER2 inhibitors), BCR-ABL inhibitor, FLT3 inhibitor, multi-kinase inhibitor (PDGFR and VEGFR inhibitor), c-kit inhibitor, FGFR inhibitor.

Examples of RAS-MAPK pathway inhibitor are BRAF inhibitor, RAF inhibitor, MEK inhibitor and ERK inhibitor.

Examples of PI3K pathway inhibitor are PI3K inhibitor or AKT inhibitor.

Examples of the molecular targeted drug are ALK inhibitor, Her family inhibitors (EGFR and HER2 inhibitors), BCR-ABL inhibitor, FLT3 inhibitor, multi-kinase inhibitor (PDGFR and VEGFR inhibitor), c-kit inhibitor, FGFR inhibitor, BRAF inhibitor, RAF inhibitor, MEK inhibitor, ERK inhibitor, PI3K inhibitor or AKT inhibitor, a BCL2 inhibitor, a CDK4/6 inhibitor, and an HDAC inhibitor and at least one pharmaceutically acceptable salts thereof.

Examples of the molecular targeted drug are

(i) AKT inhibitor: afuresertib, 8-[4-(1-aminocyclobutyl)phenyl]-9-phenyl-1,2,4-triazolo[3,4-f][1,6]naphthyridin-3(2H)-one (MK2206), trans-3-amino-1-methyl-3-(4-(3-phenyl-5H-imidazo[1,2-c]pyrido[3,4-e][1,3]oxazin-2-yl)phenyl)cyclobutanol (hereinafter referred to as “Compound 8”), capivasertib, ipatasertib, triciribine, miransertib,
(ii) ALK inhibitor: alectinib, crizotinib, lorlatinib, ceritinib, brigatinib, repotrectinib, ensartinib, alkotinib, repotrectinib, WX-0593, SAF-189s, CT-707, TQ-B3101;
(iii) Bcl2 inhibitor: venetoclax, sabutoclax, apogossypol, obatoclax, navitoclax, APG-2575, APG-1252;
(iv) BCR-ABL inhibitor: imatinib, dasatinib, ponatinib, asciminib, olverembatinib;
(v) BRAF inhibitor: encorafenib, dabrafenib, vemurafenib, sorafenib, lifirafenib, LXH-254;
(vi) CDK4/6 inhibitor: abemaciclib, ribociclib, palbociclib, lerociclib, trilaciclib, alvocidib, GLR-2007, SHR-6390, XZP-3287, BPI-1178, PF-06873600, NUV-422, FCN-437;
(vii) CDK inhibitor: seliciclib, abemaciclib, ribociclib, palbociclib, lerociclib, trilaciclib, alvocidib, mevociclib, milciclib, fadraciclib, zotiraciclib, dinaciclib, samuraciclib, dinaciclib, voruciclib, fadraciclib, FIT-039, PF-07104091, BEY-1107;
(viii) EGFR inhibitor: cetuximab, panitumumab, osimertinib, gefitinib, erlotinib, afatinib, brigatinib, sutetinib, allitinib, epitinib, xiliertinib, rociletinib, dacomitinib, simotinib, olmutinib, yinlitinib, mefatinib, alflutinib, almonertinib, icotinib, naquotinib, poziotinib, epertinib, sapitinib, cipatinib, tarloxotinib, dacomitinib, pyrotinib, pirotinib, lazertinib, varlitinib, tesevatinib, canertinib, mobocertinib, lapatinib, duligotuzumab, mobocertinib, olafertinib, zorifertinib, lifirafenib, pelitinib, DZD-9008, ASK-120067, BPI-7711, QLNC-120, ametumumab, imgatuzumab, amivantamab, seribantumab, nimotuzumab, serclutamab, depatuxizumab, tomuzotuximab, SCT-200;
(ix) Erk1/2 inhibitor: ravoxertinib, LY3214996, MK-8353, LTT462, ulixertinib, (2R)-2-(6-{5-chloro-2-[(oxan-4-yl)amino]pyrimidin-4-yl}-1-oxo-2,3-dihydro-1H-isoindol-2-yl)-N-[(1S)-1-(3-fluoro-5-methoxyphenyl)-2-hydroxyethyl]propanamide (hereinafter referred to as “Compound 9”), HH-2710;
(x) FGFR inhibitor: infigratinib, pemigatinib, erdafitinib, orantinib, derazantinib, roblitinib, rogaratinib, zoligratinib, E-7090, AZD-4547, ODM-203, ICP-192, HMPL-453, bemarituzumab;
(xi) FLT3 inhibitor: quizartinib, crenolanib, giltertinib, flysyn, mivavotinib, PHI-101, MEN-1703, FF-10101, HM-43239, E-6201, ENMD-2076;
(xii) HER2 inhibitor: lapatinib, neratinib, larotinib, sapitinib, epertinib, tucatinib (irbinitinib), BDTX-189, trastuzumab, pertuzumab, zanidatamab, zenocutuzumab, margetuximab, KN-026, BAT-8001, TAA-013, KL-A166;
(xiii) MEK inhibitor: selumetinib, trametinib, cobimetinib, binimetinib, refametinib, mirdametinib, pimasertib, HL-085, NFX-179;
(xiv) Multi-kinase inhibitor: sunitinib, nilotinib, dovitinib, axitinib, vatalinib, pazopanib, avapritinib, regorafenib, nintedanib, anlotinib, vandetanib, ponatinib, lenvatinib, famitinib, catequentinib, necuparanib, dovitinib, surufatinib, lucitanib, midostaurin, vorolanib, pirotinib;
(xv) VEGFR inhibitor: lenvatinib, bevasiranib, bevacizumab, ranibizumab, vanucizumab, navicixizumab, ramucirumab, vorolanib, BAT-5906, VGX-100, CSL-346;
(xvi) PI3K inhibitor: duvelisib, copanlisib, buparlisib, paxalisib, alpelisib, idelalisib/CAL-101, voxtalisib, zandelisib, dezapelisib, linperlisib, inavolisib, parsaclisib, eganelisib, nemiralisib, seletalisib, gedatolisib, leniolisib, tenalisib, paxalisib, pictilisib, bimiralisib, BBP-681, BGB-10188, MEN-1611, ASN-003, ACP-319;
(xvii) HDAC inhibitor: panobinostat, resminostat, abexinostat, romidepsin, belinostat, entinostat, quisinostat, pracinostat, tefinostat, mocetinostat, givinostat, dacinostat, vorinostat (SAHA), ivaltinostat, pracinostat, domatinostat, fimepinostat, tinostamustine, panobinostat, tefinostat, Remetinostat, tucidinostat, ricolinostat, resminostat, CXD-101, REC-2282;
(xviii) CD20 inhibitor: veltuzumab, rituximab, ublituximab.

Molecular targeting drugs include low-molecular-weight molecular targeting drugs, antibody molecular targeting drugs, and immune checkpoint inhibitors.

Examples of low-molecular-weight molecular targeting drugs are afuresertib, 8-[4-(1-aminocyclobutyl)phenyl]-9-phenyl-1,2,4-triazolo[3,4-f][1,6]naphthyridin-3(2H)-one (MK2206), trans-3-amino-1-methyl-3-(4-(3-phenyl-5H-imidazo[1,2-c]pyrido[3,4-e][1,3]oxazin-2-yl)phenyl)cyclobutanol, capivasertib, ipatasertib, triciribine, miransertib, lorlatinib, ceritinib, brigatinib, repotrectinib, ensartinib, alkotinib, repotrectinib, alectinib, crizotinib, lorlatinib, ceritinib, repotrectinib, ensartinib, alkotinib, repotrectinib, WX-0593, SAF-189s, CT-707, TQ-B3101, venetoclax, sabutoclax, apogossypol, obatoclax, navitoclax, APG-2575, APG-1252, imatinib, dasatinib, ponatinib, asciminib, olverembatinib, encorafenib, dabrafenib, vemurafenib, sorafenib, lifirafenib, LXH-254, abemaciclib, ribociclib, palbociclib, lerociclib, trilaciclib, alvocidib, GLR-2007, SHR-6390, XZP-3287, BPI-1178, PF-06873600, NUV-422, FCN-437, seliciclib, ribociclib, lerociclib, trilaciclib, alvocidib, mevociclib, milciclib, fadraciclib, zotiraciclib, dinaciclib, samuraciclib, dinaciclib, voruciclib, fadraciclib, FIT-039, PF-07104091, BEY-1107, osimertinib, gefitinib, erlotinib, afatinib, sutetinib, allitinib, epitinib, xiliertinib, rociletinib, dacomitinib, simotinib, olmutinib, yinlitinib, mefatinib, alflutinib, almonertinib, icotinib, naquotinib, poziotinib, epertinib, sapitinib, cipatinib, tarloxotinib, dacomitinib, pyrotinib, pirotinib, lazertinib, varlitinib, tesevatinib, canertinib, mobocertinib, lapatinib, duligotuzumab, mobocertinib, olafertinib, zorifertinib, lifirafenib, pelitinib, DZD-9008, ASK-120067, BPI-7711, QLNC-120, ravoxertinib, LY3214996, MK-8353, LTT462, ulixertinib, (2R)-2-(6-{5-chloro-2-[(oxan-4-yl)amino]pyrimidin-4-yl}-1-oxo-2,3-dihydro-1H-isoindol-2-yl)-N-[(1S)-1-(3-fluoro-5-methoxyphenyl)-2-hydroxyethyl]propanamide, HH-2710, infigratinib, pemigatinib, erdafitinib, orantinib, derazantinib, roblitinib, rogaratinib, zoligratinib, E-7090, AZD-4547, ODM-203, ICP-192, HMPL-453, quizartinib, crenolanib, giltertinib, flysyn, mivavotinib, PHI-101, MEN-1703, FF-10101, HM-43239, E-6201, ENMD-2076, lapatinib, neratinib, larotinib, sapitinib, epertinib, tucatinib (irbinitinib), BDTX-189, selumetinib, trametinib, cobimetinib, binimetinib, refametinib, mirdametinib, pimasertib, HL-085, NFX-179, sunitinib, nilotinib, dovitinib, axitinib, vatalinib, pazopanib, avapritinib, regorafenib, nintedanib, anlotinib, vandetanib, ponatinib, lenvatinib, famitinib, catequentinib, necuparanib, dovitinib, surufatinib, lucitanib, midostaurin, vorolanib, pirotinib, bevasiranib, duvelisib, copanlisib, buparlisib, paxalisib, alpelisib, idelalisib/CAL-101, voxtalisib, zandelisib, dezapelisib, linperlisib, inavolisib, parsaclisib, eganelisib, nemiralisib, seletalisib, gedatolisib, leniolisib, tenalisib, paxalisib, pictilisib, bimiralisib, BBP-681, BGB-10188, MEN-1611, ASN-003, ACP-319, panobinostat, resminostat, abexinostat, romidepsin, belinostat, entinostat, quisinostat, pracinostat, tefinostat, mocetinostat, givinostat, dacinostat, and vorinostat (SAHA) ivaltinostat, pracinostat, domatinostat, fimepinostat, tinostamustine, panobinostat, tefinostat, Remetinostat, tucidinostat, ricolinostat, resminostat, CXD-101, REC-2282, and pharmaceutically acceptable salts thereof. Preferably, low-molecular-weight molecular targeting drugs are erlotinib, osimertinib, gefitinib, anlotinib, lapatinib, neratinib, afatinib, sunitinib, ponatinib, nintedanib, vandetanib, brigatinib, erdafitinib, dasatinib, gilteritinib, alectinib, crizotinib, egoragfenib, sorafenib, trametinib, cobimetinib, binimetinib, ulixertinib, MK-2206, ideralisib, alpelisib, venetoclax, palbociclib, abemaciclib, and vorinostat.

Examples of antibody molecular targeting drugs are trastuzumab, cetuximab, bevacizumab, panitumumab, ametumumab, imgatuzumab, amivantamab, seribantumab, nimotuzumab, serclutamab, depatuxizumab, tomuzotuximab, SCT-200, pertuzumab, zanidatamab, zenocutuzumab, margetuximab, KN-026, BAT-8001, TAA-013, KL-A166, ranibizumab, vanucizumab, navicixizumab, veltuzumab, rituximab, ublituximab, and ramucirumab, preferably cetuximab.

Examples of immune checkpoint inhibitors are nivolumab, pembrolizumab, atezolizumab, durvalumab, avelumab, ipilimumab, tremelimumab, camrelizumab, sugemalimab, toripalimab, cemiplimab, genolimzumab, spartalizumab, sintilimab, tislelizumab, zimberelimab, cetrelimab, sasanlimab, dostarlimab, retifanlimab, toripalimab, balstilimab, envafolimab, TQB-2450, HLX-10, SCT-I10A, ZKAB-001, AK-105, HX-008, KN-046, MGD-013, SHR-1316, CS-1003, RRx-001 and abatacept.

The above-described Compound 8 is a compound described in Example 32 of WO 2012/137870 and can be synthesized on the basis of a production method described therein.

The above-described Compound 9 is a compound described in WO 2018/193410 and WO 2017/068412, and can be synthesized on the basis of a production method described therein.

The molecular targeting drug in the present invention is even more preferably cetuximab, MK-2206, alectinib, crizotinib, venetoclax, imatinib, dasatinib, ponatinib, dabrafenib, vemurafenib, sorafenib, palbociclib, abemaciclib, osimertinib, gefitinib, erlotinib, afatinib, brigatinib, ulixertinib, (2R)-2-(6-{5-chloro-2-[(oxan-4-yl)amino]pyrimidin-4-yl}-1-oxo-2,3-dihydro-1H-isoindol-2-yl)-N-[(1S)-1-(3-fluoro-5-methoxyphenyl)-2-hydroxyethyl]propanamide or a salt thereof, erdafitinib, giltertinib, lapatinib, neratinib, trametinib, cobimetinib, binimetinib, regorafenib, sunitinib, nintedanib, anlotinib, vandetanib, lenvatinib, alpelisib and idelalisib/CAL-101, and vorinostat (SAHA).

Cytotoxic drugs include a topoisomerase inhibitor (a topoisomerase I inhibitor, and topoisomerase II inhibitor), alkylating agent, anthracycline antibiotics, alkaloid, anti-metabolite, anti-microtubule agent, platinum-containing drug, proteasome inhibitor and thalidomide analog drug.

Examples of cytotoxic drugs are

(i) topoisomerase I inhibitor: irinotecan (SN-38), nogitecan, simmitecan, gimatecan, topotecan, cositecan, belotecan, govitecan, deruxtecan, AR-67;

(ii) topoisomerase II inhibitor: etoposide, camsirubicin, Aldoxorubicin, vosaroxin, mitoxantrone, evofosfamide, amrubicin, sobuzoxane, epirubicin, F-14512;

(iii) alkylating agent: cyclophosphamide, dacarbazine, temozolomide, nimustine, busulfan, procarbazine, melphalan, mitomycin C;

(iv) anthracycline antibiotics: doxorubicin, daunorubicin, epirubicin;

(v) alkaloid: vincristine, vinblastine, vinorelbine, eribulin;

(vi) anti-metabolite: gemcitabine, pemetrexed, 5-FU, FdUrd, trifluridine;

(vii) anti-microtubule agent: paclitaxel (paclitaxel includes derivatives such as albumin-bound paclitaxel (e.g., ABI-007) and PEG-bound paclitaxel), docetaxel, cabazitaxel, avanbulin, fluorapacin, mertansine;

(viii) platinum-containing drug: cisplatin, carboplatin, oxaliplatin, nedaplatin;

(ix) proteasome inhibitor: bortezomib, ixazomib, marizomib, carfilzomib, LXE-408.

Anti-metabolites include a purine anti-metabolite, a pyrimidine anti-metabolite, and an antifolate.

Examples of purine antimetabolites are fludarabine, cladribine, and nelarabine.

Examples of pyrimidine anti-metabolites are 5-fluorouracil (5-FU), tegafur/gimeracil/oteracil potassium (TS-1 or S-1, trade name: “TS-1”), tegafur/uracil (UFT, trade name: “UFT”), trifluridine/tipiracil hydrochloride (TAS-102, trade name: “LONSURF”), capecitabine, doxifluridine, 5-fluoro-2′-deoxyuridine (FdUrd), gemcitabine, and cytarabine.

Examples of antifolates are pemetrexed and methotrexate.

The cytotoxic drug in the present invention is even more preferably irinotecan (SN-38), etoposide, cyclophosphamide, doxorubicin, gemcitabine, pemetrexed, 5-FU, FdUrd, FTD, paclitaxel, cisplatin, oxaliplatin, and bortezomib.

The AC in the present invention is even more preferably cetuximab, MK-2206, alectinib, crizotinib, venetoclax, imatinib, dasatinib, ponatinib, dabrafenib, vemurafenib, sorafenib, palbociclib, abemaciclib, osimertinib, gefitinib, erlotinib, afatinib, brigatinib, ulixertinib, (2R)-2-(6-{5-chloro-2-[(oxan-4-yl)amino]pyrimidin-4-yl}-1-oxo-2,3-dihydro-1H-isoindol-2-yl)-N-[(1S)-1-(3-fluoro-5-methoxyphenyl)-2-hydroxyethyl]propanamide or a salt thereof, erdafitinib, giltertinib, lapatinib, neratinib, trametinib, cobimetinib, binimetinib, regorafenib, sunitinib, nintedanib, anlotinib, vandetanib, lenvatinib, alpelisib and idelalisib/CAL-101, vorinostat (SAHA), irinotecan (SN-38), etoposide, cyclophosphamide, doxorubicin, gemcitabine, pemetrexed, 5-FU, FdUrd, FTD, paclitaxel, cisplatin, oxaliplatin, and bortezomib.

As known to persons skilled in the art, even medicines excellent in antitumor effect may inflict additional suffering to patients due to their side effects. The combination drug of the present invention can reduce the dose and dosing frequency of a medicine by the enhancement of the antitumor effect and can consequently be effective for the suppression of side effects.

The malignant tumor that can be treated with the combination drug of the present invention is not particularly limited, and examples thereof include epithelial cancer (respiratory cancer, gastrointestinal cancer, genital cancer, cancer of the secretory system, breast cancer, etc.), mesothelioma, sarcoma, hematopoietic tumor, tumors of the central nervous system, retinoblastoma, and tumors of the peripheral nervous system.

Specific examples of the respiratory cancer include lung cancer (non-small cell lung cancer, small-cell lung cancer, bronchogenic cancer, etc.). Specific examples of the gastrointestinal cancer include esophagus cancer, gastric cancer, gastrointestinal stromal tumors, duodenum cancer, liver cancer, hepatocellular cancer, biliary tract cancer (gallbladder cancer, cholangiocarcinoma, intrahepatic cholangiocarcinoma, extrahepatic cholangiocarcinoma, etc.), pancreatic cancer, and colorectal cancer (colon cancer, rectum cancer, etc.). Specific examples of the genital cancer include ovarian cancer, uterine cancer (cervical cancer, endometrial cancer, etc.), renal cancer (Wilms' tumor, etc.), bladder cancer, prostate cancer (urothelial carcinoma, testicular cancer etc.), and testicular tumor. Specific examples of the cancer of the secretory system include thyroid cancer. Specific examples of the sarcoma include bone or soft tissue tumor, and angiosarcoma. Specific examples of the hematopoietic tumor include leukemia (chronic myeloid leukemia, acute myeloid leukemia, chronic lymphocytic leukemia, acute lymphoblastic leukemia, etc.), malignant lymphoma (Hodgkin's lymphoma, small lymphocytic lymphoma, follicular lymphoma, cutaneous T-cell lymphoma, mixed-cell type lymphoma, histiocytic lymphoma, Burkitt's lymphoma, mycosis fungoides, mantle cell lymphoma etc.), and multiple myeloma. Specific examples of the tumors of the central nervous system include head and neck cancer, brain tumor and neuroblastoma. Specific examples of the tumors of the peripheral nervous system include skin cancer (melanoma, etc.).

The malignant tumor to be treated in the present invention is even more preferably respiratory cancer such as lung cancer, esophagus cancer, gastric cancer, biliary tract cancer (gallbladder cancer, cholangiocarcinoma, intrahepatic cholangiocarcinoma, or extrahepatic cholangiocarcinoma), endometrial cancer, bladder cancer, breast cancer, osteosarcoma, soft tissue sarcoma, multiple myeloma, or brain tumor, and particularly preferably gastric cancer, biliary tract cancer (gallbladder cancer, cholangiocarcinoma, intrahepatic cholangiocarcinoma, or extrahepatic cholangiocarcinoma), endometrial cancer, bladder cancer, or brain tumor.

When the AC is cetuximab, the malignant tumor to be treated in the present invention is more preferably the tumors of the central nervous system and colorectal cancer, and even more preferably head and neck cancer and colorectal cancer.

When the AC is alectinib, the malignant tumor to be treated in the present invention is more preferably lung cancer, and even more preferably non-small cell lung cancer.

When the AC is crizotinib, the malignant tumor to be treated in the present invention is more preferably lung cancer, and even more preferably non-small cell lung cancer.

When the AC is venetoclax, the malignant tumor to be treated in the present invention is more preferably malignant lymphoma, and even more preferably chronic lymphocytic leukemia.

When the AC is imatinib, the malignant tumor to be treated in the present invention is more preferably malignant lymphoma and gastric, and even more preferably chronic lymphocytic leukemia, acute lymphoblastic leukemia, gastrointestinal stromal tumors.

When the AC is dasatinib, the malignant tumor to be treated in the present invention is more preferably leukemia, and even more preferably chronic myeloid leukemia and acute lymphoblastic leukemia.

When the AC is dabrafenib, the malignant tumor to be treated in the present invention is more preferably skin cancer, and even more preferably melanoma.

When the AC is vemurafenib, the malignant tumor to be treated in the present invention is more preferably skin cancer, and even more preferably melanoma.

When the AC is sorafenib, the malignant tumor to be treated in the present invention is more preferably liver cancer, hepatocellular cancer renal cancer, skin cancer, and even more preferably hepatocellular cancer, renal cancer and thyroid cancer.

When the AC is palbociclib, the malignant tumor to be treated in the present invention is more preferably breast cancer.

When the AC is abemaciclib, the malignant tumor to be treated in the present invention is more preferably breast cancer.

When the AC is osimertinib, the malignant tumor to be treated in the present invention is more preferably lung cancer, and even more preferably non-small cell lung cancer.

When the AC is gefitinib, the malignant tumor to be treated in the present invention is more preferably lung cancer, and even more preferably non-small cell lung cancer.

When the AC is erlotinib, the malignant tumor to be treated in the present invention is more preferably lung cancer and pancreatic cancer, and even more preferably non-small cell lung cancer and pancreatic cancer.

When the AC is afatinib, the malignant tumor to be treated in the present invention is more preferably lung cancer, and even more preferably non-small cell lung cancer.

When the AC is brigatinib, the malignant tumor to be treated in the present invention is more preferably lung cancer, and even more preferably non-small cell lung cancer.

When the AC is erdafitinib, the malignant tumor to be treated in the present invention is more preferably prostate cancer, and even more preferably urothelial carcinoma.

When the AC is lapatinib, the malignant tumor to be treated in the present invention is more preferably breast cancer.

When the AC is neratinib, the malignant tumor to be treated in the present invention is more preferably breast cancer.

When the AC is trametinib, the malignant tumor to be treated in the present invention is more preferably skin cancer and lung cancer, and even more preferably melanoma and non-small cell lung cancer.

When the AC is cobimetinib, the malignant tumor to be treated in the present invention is more preferably skin cancer, and even more preferably melanoma.

When the AC is binimetinib, the malignant tumor to be treated in the present invention is more preferably skin cancer, and even more preferably melanoma.

When the AC is sunitinib, the malignant tumor to be treated in the present invention is more preferably gastric cancer, renal cancer and pancreatic cancer, and even more preferably gastrointestinal stromal tumors, renal cancer and pancreatic cancer.

When the AC is nintedanib, the malignant tumor to be treated in the present invention is more preferably lung cancer, and even more preferably non-small cell lung cancer.

When the AC is vandetanib, the malignant tumor to be treated in the present invention is more preferably thyroid cancer.

When the AC is ponatinib, the malignant tumor to be treated in the present invention is more preferably leukemia, and even more preferably chronic myeloid leukemia and acute lymphoblastic leukemia.

When the AC is lenvatinib, the malignant tumor to be treated in the present invention is more preferably skin cancer, renal cancer, liver cancer and hepatocellular cancer, and even more preferably melanoma, renal cancer and acute hepatocellular cancer.

When the AC is alpelisib, the malignant tumor to be treated in the present invention is more preferably breast cancer.

When the AC is idelalisib, the malignant tumor to be treated in the present invention is more preferably leukemia and malignant lymphoma, and even more preferably chronic lymphocytic leukemia, follicular lymphoma and small lymphocytic lymphoma.

When the AC is sorafenib, the malignant tumor to be treated in the present invention is more preferably hepatocellular cancer, renal cancer and thyroid cancer.

When the AC is vorinostat, the malignant tumor to be treated in the present invention is more preferably malignant lymphoma, and even more preferably cutaneous T-cell lymphoma.

When the AC is etoposide, the malignant tumor to be treated in the present invention is more preferably prostate cancer and lung cancer, and even more preferably testicular cancer and small cell lung cancer.

When the AC is cyclophosphamide, the malignant tumor to be treated in the present invention is more preferably malignant lymphoma, multiple myeloma, leukemia, the tumors of the central nervous system, ovarian cancer, retinoblastoma and breast cancer, and even more preferably Hodgkin's disease, lymphocytic lymphoma, mixed-cell type lymphoma, histiocytic lymphoma, Burkitt's lymphoma, mycosis fungoides, multiple myeloma, leukemia, neuroblastoma, ovarian cancer, retinoblastoma and breast cancer.

When the AC is doxorubicin, the malignant tumor to be treated in the present invention is more preferably breast cancer, leukemia, lymphoma, renal cancer, the tumors of the central nervous system, sarcoma, ovarian cancer, bladder cancer, skin cancer, gastric cancer and lung cancer, and even more preferably acute lymphoblastic leukemia, acute myeloblastic leukemia, Hodgkin lymphoma, Non-Hodgkin lymphoma, breast cancer, Wilms' tumor, neuroblastoma, bone tumor, soft tissue tumor, ovarian cancer, bladder cancer, thyroid cancer, gastric cancer, bronchogenic cancer.

When the AC is gemcitabine, the malignant tumor to be treated in the present invention is more preferably ovarian cancer, breast cancer, lung cancer, pancreatic cancer, and even more preferably ovarian cancer, breast cancer, non-small cell lung cancer, pancreatic cancer.

When the AC is pemetrexed, the malignant tumor to be treated in the present invention is more preferably lung cancer and mesothelioma, and even more preferably non-small cell lung cancer and mesothelioma.

When the AC is 5-FU, the malignant tumor to be treated in the present invention is more preferably colorectal cancer, breast cancer, gastric cancer and pancreatic cancer, and even more preferably colon cancer, rectum cancer, breast cancer, gastric cancer and pancreatic cancer.

When the AC is trifluridine, the malignant tumor to be treated in the present invention is more preferably colorectal cancer and gastric cancer.

When the AC is paclitaxel, the malignant tumor to be treated in the present invention is more preferably ovarian cancer, breast cancer, lung cancer, sarcoma, the tumors of the central nervous system and gastrointestinal cancer, and even more preferably ovarian cancer, breast cancer, non-small cell lung cancer, angiosarcoma, head and neck cancer and esophagus cancer.

When the AC is oxaliplatin, the malignant tumor to be treated in the present invention is more preferably colorectal cancer, pancreatic cancer and gastric cancer.

When the AC is cisplatin, the malignant tumor to be treated in the present invention is more preferably prostate cancer, ovarian cancer, bladder cancer, the tumors of the central nervous system, lung cancer, gastrointestinal cancer, mesothelioma, sarcoma, biliary tract cancer and lymphoma, and even more preferably testicular tumor, prostate cancer, ovarian cancer, bladder cancer, head and neck cancer, non-small cell lung cancer, small cell lung cancer, gastric cancer, esophagus cancer, mesothelioma, bone tumor, biliary tract cancer and lymphoma.

When the AC is bortezomib, the malignant tumor to be treated in the present invention is more preferably multiple myeloma and lymphoma, and even more preferably multiple myeloma and mantle cell lymphoma.

When the AC is compound 9, the malignant tumor to be treated in the present invention is more preferably lung cancer, ovarian cancer, cervical cancer, uterine/endometrial cancer, lymphoma, leukemia, myeloma, breast cancer, skin cancer, gastric cancer, esophageal cancer, colon cancer, colorectal cancer, kidney cancer, liver cancer, bile duct cancer, urinary bladder cancer, soft-tissue (bone cancer and other sarcoma) cancer, head and neck cancer, prostate cancer, thyroid cancer and pancreatic cancer, which were found to be sensitive to the combination.

Examples of ACs that can be preferably used in the combination drug of the present invention for gastrointestinal cancer to be treated include an AKT inhibitor, ALK inhibitor, CDK inhibitor, ERK1/2 inhibitor, HER2 inhibitor, MEK inhibitor, multi-kinase inhibitor, PI3K inhibitor, topoisomerase I inhibitor, anthracycline antibiotics, anti-metabolite, antimicrotubule agent, platinum-containing drug. Such an AC is preferably selected from the group consisting of cetuximab, MK2206, alectinib, abemaciclib, ulixertinib, (2R)-2-(6-{5-chloro-2-[(oxan-4-yl)amino]pyrimidin-4-yl}-1-oxo-2,3-dihydro-1H-isoindol-2-yl)-N-[(1S)-1-(3-fluoro-5-methoxyphenyl)-2-hydroxyethyl]propanamide, lapatinib, neratinib, trametinib, cobimetinib, binimetinib, nintedanib, anlotinib, sunitinib, alpelisib, SN38, doxorubicin, gemcitabine, 5-FU, FTD, paclitaxel, oxaliplatin or cisplatin.

Examples of ACs that can be preferably used in the combination drug of the present invention for lung cancer to be treated include an AKT inhibitor, ALK inhibitor, BCR-ABL inhibitor, CDK4/6 inhibitor, CDK inhibitor, EGFR inhibitor, ERK1/2 inhibitor, FGFR inhibitor, FLT3 inhibitor, HER2 inhibitor, MEK inhibitor, multi-kinase inhibitor, PI3K inhibitor, HDAC inhibitor, anthracycline antibiotics, anti-metabolite, antimicrotubule agent, platinum-containing drug. Such an AC is preferably selected from the group consisting of MK2206, crizotinib, dasatinib, palbociclib, abemaciclib, osimertinib, gefitinib, erlotinib, afatinib, brigatinib, ulixertinib, (2R)-2-(6-{5-chloro-2-[(oxan-4-yl)amino]pyrimidin-4-yl}-1-oxo-2,3-dihydro-1H-isoindol-2-yl)-N-[(1S)-1-(3-fluoro-5-methoxyphenyl)-2-hydroxyethyl]propenamide, erdafitinib, gilteritinib, lapatinib, trametinib, cobimetinib, binimetinib, nintedanib, anlotinib, vandetanib, sunitinib, ponatinib, alpelisib, vorinostat (SAHA), doxorubicin, gemcitabine, paclitaxel, or oxaliplatin.

Examples of ACs that can be preferably used in the combination drug of the present invention for urinary cancer such as bladder cancer and prostate cancer to be treated include a Bcl2 inhibitor, BCR-ABL inhibitor, EGFR inhibitor, ERK1/2 inhibitor, FGFR inhibitor, MEK inhibitor, Multi-kinase inhibitor, PI3K inhibitor, HDAC inhibitor, topoisomerase I inhibitor, topoisomerase II inhibitor, anthracycline antibiotics, anti-metabolite, antimicrotubule agent and platinum-containing drug. Such an AC is preferably selected from the group consisting of venetoclax, dasatinib, osimertinib, gefitinib, erlotinib, afatinib, brigatinib, ulixertinib, (2R)-2-(6-{5-chloro-2-[(oxan-4-yl)amino]pyrimidin-4-yl}-1-oxo-2,3-dihydro-1H-isoindol-2-yl)-N-[(1S)-1-(3-fluoro-5-methoxyphenyl)-2-hydroxyethyl]propenamide, erdafitinib, trametinib, regorafenib, anlotinib, vandetanib, sunitinib, ponatinib, alpelisib, vorinostat (SAHA), SN-38, etoposide, doxorubicin, FTD, paclitaxel, oxaliplatin and cisplatin.

Examples of ACs that can be preferably used in the combination drug of the present invention for cancer that grows with sex hormones, such as ovarian cancer and breast cancer to be treated include an AKT inhibitor, Bcl2 inhibitor, CDK4/6 inhibitor, CDK inhibitor, EGFR inhibitor, multi-kinase inhibitor and PI3K inhibitor. Such an AC is preferably selected from the group consisting of MK2206, venetoclax, palbociclib, abemaciclib, afatinib, brigatinib, nintedanib, (2R)-2-(6-{5-chloro-2-[(oxan-4-yl)amino]pyrimidin-4-yl}-1-oxo-2,3-dihydro-1H-isoindol-2-yl)-N-[(1S)-1-(3-fluoro-5-methoxyphenyl)-2-hydroxyethyl]propenamide, vandetanib and idelalisib/CAL101.

Examples of ACs that can be preferably used in the combination drug of the present invention for Leukemia to be treated include an AKT inhibitor, ALK inhibitor, Bcl2 inhibitor, CDK4/6 inhibitor, CDK inhibitor, EGFR inhibitor, FLT3 inhibitor, multi-kinase inhibitor, PI3K inhibitor and RAF inhibitor. Such an AC is preferably selected from the group consisting of MK2206, alectinib, crizotinib, venetoclax, palbociclib, abemaciclib, erlotinib, afatinib, brigatinib, gilteritinib, sunitinib, nintedanib, vandetanib, (2R)-2-(6-{5-chloro-2-[(oxan-4-yl)amino]pyrimidin-4-yl}-1-oxo-2,3-dihydro-1H-isoindol-2-yl)-N-[(1S)-1-(3-fluoro-5-methoxyphenyl)-2-hydroxyethyl]propenamide, idelalisib/CAL-101 and sorafenib.

Examples of ACs that can be preferably used in the combination drug of the present invention for colorectal cancer to be treated include EGFR inhibitor. Such an AC is preferably cetuximab or (2R)-2-(6-{5-chloro-2-[(oxan-4-yl)amino]pyrimidin-4-yl}-1-oxo-2,3-dihydro-1H-isoindol-2-yl)-N-[(1S)-1-(3-fluoro-5-methoxyphenyl)-2-hydroxyethyl]propanamide.

In an embodiment, the combination drug according to the present invention has an effect on cancer that harbors the KRAS mutation. For example, the combination drug has a higher effect on cancers with a high frequency of the KRAS mutation, such as pancreatic cancer, colorectal cancer, and lung cancer. The combination drug according to the present invention has a higher effect in vivo on cancers that carry the KRAS mutation than each drug alone. Thus, cancers that carry the KRAS mutation, on which neither molecular targeted drugs nor cytotoxic drugs are effective, are expected to be sensitive to the combination drug according to the present invention. Additionally, the compound represented by formula (I) can enhance the efficacy of the molecular targeted drugs and the efficacy of cytotoxic drugs on both cancers that carry the KRAS mutation and cancers free of the KRAS mutation. The driver mutation of KRAS gene is, for example, but is not limited to, at least one member selected from the group consisting of G12A, G12C, G12F, G12R, G12S, G12V, G13C, G13D, A59G, Q61H, Q61K, Q61L, and A146T.

In the combination drug of the present invention, a compound represented by formula (I) or the pharmaceutically acceptable salt thereof, and at least one AC may be separately formulated in a plurality of preparations or may be collectively formulated in a single preparation. Also, the combination drug of the present invention may further contain an active ingredient other than the compound represented by formula (I) or the pharmaceutically acceptable salt thereof and at least one AC, and is preferably a combination drug containing only the compound represented by formula (I) or the pharmaceutically acceptable salt thereof, and at least one AC as active ingredients.

When the compound represented by formula (I) or the pharmaceutically acceptable salt thereof and at least one AC are contained as active ingredients in preparations, a pharmaceutical carrier can be added to each active ingredient, if required, thereby forming various suitable dosage forms according to prevention and treatment purposes. Examples of the dosage form include oral preparations, injections, suppositories, ointments, and patches. Oral preparations are preferable. The oral preparations can be forms such as tablets, capsules, granules, powders, and syrups and are not particularly limited. Such dosage forms can be manufactured by methods conventionally known to persons skilled in the art. Preparations or pharmaceutical compositions can be supplemented with a suitable carrier such as an excipient, diluent, bulking agent, or disintegrant according to dosage forms.

The daily dose of the combination drug may vary depending on the condition, body weight, age, and sex of a patient, etc., and cannot be generalized. Usually, the daily dose of the compound represented by formula (I) or the pharmaceutically acceptable salt thereof is approximately 0.001 to 5000 mg, preferably approximately 0.01 to 2500 mg, and more preferably approximately 0.1 to 1000 mg, and at least one AC, is approximately 0.001 to 5000 mg, preferably approximately 0.01 to 3000 mg, and more preferably approximately 0.1 to 2500 mg, per adult (body weight: 60 kg).

In the case of administering each day the daily dose of the compound represented by formula (I) or the pharmaceutically acceptable salt thereof, the compound represented by formula (I) or the pharmaceutically acceptable salt thereof is administered at approximately 0.001 to 5000 mg per day, preferably 0.01 to 2500 mg per day, more preferably 0.1 to 2000 mg per day, and even more preferably 1 to 1000 mg per day.

In the case of administering every other day the daily dose of the compound represented by formula (I) or the pharmaceutically acceptable salt thereof, is administered at approximately 0.01 to 5000 mg per day, preferably 0.1 to 2500 mg per day, more preferably 1 to 1500 mg per day, and even more preferably 2 to 1000 mg per day.

In the combination drug of the present invention, the daily dose of the AC may vary depending on the condition, body weight, age, and sex of a patient, etc., and cannot be generalized.

When the AC is cetuximab, AC is administered at approximately 10 to 500 mg/m² per dose, preferably 50 to 400 mg/m² per dose, and more preferably 250 to 400 mg/m² per dose. In another aspect, cetuximab is administered at approximately 10 to 400 mg/m² per dose, preferably 50 to 250 mg/m² per dose, and more preferably 100 to 250 mg/m² per dose.

When the AC is MK-2206, MK-2206 is administered at approximately 10 to 500 mg per day, preferably 50 to 300 mg per day, more preferably 80 to 210 mg per day, and even more preferably 90 to 200 mg per day.

When the AC is alectinib, AC is administered at approximately 100 to 1500 mg per day, preferably 150 to 1200 mg per day, more preferably 600 to 1200 mg per day, and even more preferably 600, 750, 900, 1050, and 1200 mg per day.

When the AC is alectinib, AC is administered at approximately 100 to 1500 mg per day, preferably 150 to 1200 mg per day, more preferably 600 to 1200 mg per day, and even more preferably 600, 750, 900, 1050, and 1200 mg per day.

When the AC is crizotinib, AC is administered at approximately 100 to 1500 mg per day, preferably 100 to 1000 mg per day, more preferably 200 to 500 mg per day, and even more preferably 200, 250, 400, 450, and 500 mg per day.

When the AC is venetoclax, AC is administered at approximately 10 to 500 mg per day, preferably 10 to 100 mg per day, more preferably 10 to 50 mg per day, and even more preferably 10 and 20 mg per day. In another aspect, venetoclax is administered at approximately 10 to 500 mg per day, preferably 100 to 500 mg per day, more preferably 200 to 400 mg per day, and even more preferably 200, 210, 220, 230, 240, 250, 260, 270, 280, 290, 300, 310, 320, 330, 340, 350, 360, 370, 380, 390 and 400 mg per day.

When the AC is imatinib, AC is administered at approximately 10 to 1000 mg per day, preferably 100 to 800 mg per day, more preferably 400 to 800 mg per day, and even more preferably 400, 600, and 800 mg per day. In another aspect, imatinib is administered at approximately 10 to 1000 mg per day, preferably 200 to 600 mg per day, more preferably 200 to 400 mg per day, and even more preferably 200 and 400 mg per day. In another aspect, imatinib is administered at approximately 10 to 1000 mg per day, preferably 100 to 200 mg per day, and even more preferably 100 and 200 mg per day.

When the AC is dasatinib, AC is administered at approximately 10 to 200 mg per day, preferably 10 to 150 mg per day, more preferably 50 to 140 mg per day, and even more preferably 50, 60, 70, 80, 90, 100, 110, 120, 130 and 140 mg per day. In another aspect, venetoclax is administered at approximately 10 to 200 mg per day, preferably 10 to 100 mg per day, more preferably 50 to 100 mg per day, and even more preferably 50, 60, 70, 80, 90 and 100 mg per day.

When the AC is dabrafenib, AC is administered at approximately 10 to 200 mg per day, preferably 50 to 200 mg per day, more preferably 50 to 150 mg per day, and even more preferably 50, 75, 100, 125 and 150 mg per day.

When the AC is vemurafenib, AC is administered at approximately 100 to 3000 mg per day, preferably 400 to 2000 mg per day, more preferably 480 to 1920 mg per day, and even more preferably, 480, 720, 960, 1200, 1440, 1680 and 1920 mg per day.

When the AC is sorafenib, AC is administered at approximately 100 to 1500 mg per day, preferably 200 to 1000 mg per day, more preferably 200 to 800 mg per day, and even more preferably, 200, 400, 600, and 800 mg per day.

When the AC is palbociclib, AC is administered at approximately 10 to 200 mg per day, preferably 50 to 150 mg per day, more preferably 75 to 125 mg per day, and even more preferably, 75, 100, and 125 mg per day.

When the AC is abemaciclib, AC is administered at approximately 10 to 300 mg per day, preferably 20 to 250 mg per day, more preferably 50 to 200 mg per day, and even more preferably 50, 100, 150 and 200 mg per day.

When the AC is osimertinib, AC is administered at approximately 10 to 200 mg per day, preferably 20 to 100 mg per day, more preferably 40 to 80 mg per day, and even more preferably 40 and 80 mg per day.

When the AC is gefitinib, AC is administered at approximately 10 to 500 mg per day, preferably 20 to 300 mg per day, more preferably 100 to 250 mg per day, and even more preferably 250 mg per day.

When the AC is erlotinib, AC is administered at approximately 10 to 300 mg per day, preferably 20 to 200 mg per day, more preferably 25 to 150 mg per day, and even more preferably 25, 50. 75, 100, 125 and 150 mg per day. In another aspect, erlotinib is administered at approximately 10 to 200 mg per day, preferably 25 to 100 mg per day, and even more preferably 25, 50, 75 and 100 mg per day.

When the AC is afatinib, AC is administered at approximately 1 to 100 mg per day, preferably 5 to 50 mg per day, more preferably 20 to 40 mg per day, and even more preferably 20, 30 and 40 mg per day. In another aspect, afatinib is administered at approximately 1 to 50 mg per day, preferably 20 to 30 mg per day, and even more preferably 20 and 30 mg per day.

When the AC is brigatinib, AC is administered at approximately 10 to 300 mg per day, preferably 20 to 200 mg per day, more preferably 30 to 180 mg per day, and even more preferably 30, 60, 90, 120, 150 and 180 mg per day. In another aspect, brigatinib is administered at approximately 10 to 100 mg per day, preferably 30 to 90 mg per day, and even more preferably 30, 60 and 90 mg per day.

When the AC is ulixertinib, AC is administered at approximately 20 to 1000 mg per day, preferably 50 to 800 mg per day, more preferably 100 to 600 mg per day.

When the AC is erdafitinib, AC is administered at approximately 0.5 to 50 mg per day, preferably 1 to 20 mg per day, more preferably 3 to 9 mg per day, and even more preferably 3, 4, 5, 6, 7, 8 and 9 mg per day. In another aspect, erdafitinib is administered at approximately 0.1 to 40 mg per day, preferably 1 to 15 mg per day, more preferably 3 to 8 mg per day, and even more preferably 3, 4, 5, 6, 7 and 8 mg per day.

When the AC is gilteritinib, AC is administered at approximately 10 to 300 mg per day, preferably 20 to 200 mg per day, more preferably 40 to 120 mg per day, and even more preferably 40, 80 and 120 mg per day.

When the AC is lapatinib, AC is administered at approximately 100 to 3000 mg per day, preferably 200 to 2000 mg per day, more preferably 250 to 1500 mg per day, and even more preferably 250, 500, 750, 1000, 1250 and 1500 mg per day. In another aspect, lapatinib is administered at approximately 100 to 2500 mg per day, preferably 200 to 1500 mg per day, more preferably 250 to 1250 mg per day, and even more preferably 250, 500, 750, 1000 and 1250 mg per day.

When the AC is neratinib, AC is administered at approximately 10 to 500 mg per day, preferably 20 to 300 mg per day, more preferably 40 to 240 mg per day, and even more preferably 40, 80, 120, 160, 200 and 240 mg per day. In another aspect, neratinib is administered at approximately 1 to 150 mg per day, preferably 10 to 100 mg per day, more preferably 40 to 80 mg per day, and even more preferably 40 and 80 mg per day.

When the AC is trametinib, AC is administered at approximately 0.1 to 10 mg per day, preferably 0.2 to 5 mg per day, more preferably 0.5 to 2 mg per day, and even more preferably 0.5, 1, 1.5 and 2 mg per day.

When the AC is cobimetinib, AC is administered at approximately 1 to 200 mg per day, preferably 5 to 100 mg per day, more preferably 20 to 60 mg per day, and even more preferably 20, 40 and 60 mg per day.

When the AC is binimetinib, AC is administered at approximately 1 to 200 mg per day, preferably 10 to 100 mg per day, more preferably 15 to 90 mg per day, and even more preferably 15, 30, 45, 60, 75 and 90 mg per day. In another aspect, binimetinib is administered at approximately 1 to 100 mg per day, preferably 10 to 80 mg per day, more preferably 15 to 60 mg per day, and even more preferably 15, 30, 45 and 60 mg per day.

When the AC is binimetinib, AC is administered at approximately 10 to 500 mg per day, preferably 20 to 200 mg per day, more preferably 40 to 160 mg per day, and even more preferably 40, 80, 120 and 160 mg per day.

When the AC is sunitinib, AC is administered at approximately 1 to 200 mg per day, preferably 5 to 100 mg per day, more preferably 12.5 to 50 mg per day, and even more preferably 12.5, 25, 37.5 and 50 mg per day. In another aspect, sunitinib is administered at approximately 1 to 150 mg per day, preferably 5 to 40 mg per day, more preferably 12.5 to 37.5 mg per day, and even more preferably 12.5, 25 and 37.5 mg per day.

When the AC is nintedanib, AC is administered at approximately 10 to 1000 mg per day, preferably 20 to 500 mg per day, more preferably 100 to 400 mg per day, and even more preferably 100, 150, 200, 250, 300, 350 and 400 mg per day. In another aspect, nintedanib is administered at approximately 10 to 500 mg per day, preferably 50 to 400 mg per day, more preferably 100 to 300 mg per day, and even more preferably 100, 150, 200, 250 and 300 mg per day. In another aspect, nintedanib is administered at approximately 10 to 300 mg per day, preferably 50 to 250 mg per day, more preferably 100 to 200 mg per day, and even more preferably 100, 150 and 200 mg per day.

When the AC is anlotinib, AC is administered at approximately 0.1 to 30 mg per day, preferably 0.1 to 20 mg per day, more preferably 1 to 15 mg per day, and even more preferably 5 to 12 mg per day.

When the AC is vandetanib, AC is administered at approximately 10 to 500 mg per day, preferably 20 to 400 mg per day, more preferably 100 to 300 mg per day, and even more preferably 100, 200 and 300 mg per day. In another aspect, nintedanib is administered at approximately 10 to 400 mg per day, preferably 50 to 300 mg per day, more preferably 100 to 200 mg per day, and even more preferably 100 and 200 mg per day.

When the AC is ponatinib, AC is administered at approximately 1 to 100 mg per day, preferably 10 to 50 mg per day, more preferably 15 to 45 mg per day, and even more preferably 15, 30 and 45 mg per day. In another aspect, ponatinib is administered at approximately 1 to 50 mg per day, preferably 5 to 40 mg per day, more preferably 15 to 30 mg per day, and even more preferably 15 and 30 mg per day.

When the AC is lenvatinib, AC is administered at approximately 1 to 100 mg per day, preferably 2 to 50 mg per day, more preferably 4 to 24 mg per day, and even more preferably 4, 8, 10, 12, 14, 16, 18, 20, 22 and 24 mg per day. In another aspect, lenvatinib is administered at approximately 1 to 50 mg per day, preferably 2 to 25 mg per day, more preferably 4 to 18 mg per day, and even more preferably 4, 8, 10, 12, 14, 16 and 18 mg per day. In another aspect, lenvatinib is administered at approximately 1 to 40 mg per day, preferably 2 to 20 mg per day, more preferably 4 to 14 mg per day, and even more preferably 4, 8, 10, 12 and 14 mg per day. In another aspect, lenvatinib is administered at approximately 1 to 20 mg per day, preferably 2 to 15 mg per day, more preferably 4 to 10 mg per day, and even more preferably 4, 8 and 10 mg per day.

When the AC is alpelisib, AC is administered at approximately 10 to 1000 mg per day, preferably 50 to 500 mg per day, more preferably 100 to 300 mg per day, and even more preferably 100, 150, 200, 250 and 300 mg per day.

When the AC is idelalisib, AC is administered at approximately 1 to 1000 mg per day, preferably 10 to 500 mg per day, more preferably 50 to 300 mg per day, and even more preferably 50, 100, 150, 200, 250 and 300 mg per day.

When the AC is sorafenib, AC is administered at approximately 10 to 2000 mg per day, preferably 100 to 1000 mg per day, more preferably 200 to 800 mg per day, and even more preferably 200, 400, 600 and 800 mg per day.

When the AC is vorinostat, AC is administered at approximately 10 to 1000 mg per day, preferably 50 to 500 mg per day, more preferably 100 to 400 mg per day, and even more preferably 100, 200, 300 and 400 mg per day. In another aspect, vorinostat is administered at approximately 10 to 500 mg per day, preferably 50 to 400 mg per day, more preferably 100 to 300 mg per day, and even more preferably 100, 200 and 300 mg per day.

When the AC is SN-38, AC is administered at approximately 10 to 300 mg per day, preferably 50 to 200 mg per day, more preferably 100 to 150 mg per day. In another aspect, SN-38 is administered at approximately 30 to 500 mg per day, preferably 100 to 300 mg per day, more preferably 150 to 200 mg per day. In another aspect, SN-38 is administered at approximately 50 to 1000 mg per day, preferably 100 to 500 mg per day, more preferably 250 to 350 mg per day.

When the AC is etoposide, AC is administered at approximately 1 to 500 mg per day, preferably 10 to 300 mg per day, more preferably 50 to 100 mg per day.

When the AC is cyclophosphamide, AC is administered at approximately 1 to 200 mg/kg per dose, preferably 10 to 100 mg/m² per dose, more preferably 25 to 50 mg/kg per dose, and even more preferably 40 to 50 mg/kg per dose. In another aspect, cyclophosphamide is administered at approximately 1 to 50 mg/kg per dose, preferably 5 to 20 mg/kg per dose, more preferably 10 to 15 mg/kg per dose. In another aspect, cyclophosphamide is administered at approximately 0.1 to 15 mg/kg per dose, preferably 0.5 to 10 mg/kg per dose, more preferably 3 to 5 mg/kg per dose. In another aspect, cyclophosphamide is administered at approximately 0.01 to 15 mg/kg per dose, preferably 0.1 to 10 mg/kg per dose, more preferably 1 to 5 mg/kg per dose.

When the AC is doxorubicin, AC is administered at approximately 5 to 200 mg/m² per dose, preferably 20 to 100 mg/m² per dose, more preferably 50 to 80 mg/m² per dose, and even more preferably 60 to 75 mg/m² per dose. In another aspect, doxorubicin is administered at approximately 1 to 300 mg/m² per dose, preferably 5 to 100 mg/m² per dose, more preferably 40 to 75 mg/m² per dose.

When the AC is gemcitabine, AC is administered at approximately 100 to 3000 mg/m² per dose, preferably 250 to 2000 mg/m² per dose, more preferably 500 to 1500 mg/m² per dose, and even more preferably 1000 to 1250 mg/m² per dose. In another aspect, doxorubicin is administered at approximately 50 to 3000 mg/m² per dose, preferably 250 to 1500 mg/m² per dose, more preferably 500 to 1000 mg/m² per dose.

When the AC is pemetrexed, AC is administered at approximately 50 to 1000 mg/m² per dose, preferably 100 to 800 mg/m² per dose, more preferably 250 to 750 mg/m² per dose, and even more preferably 300 to 500 mg/m² per dose.

When the AC is 5-FU, AC is administered at approximately 10 to 1000 mg/m² per dose, preferably 50 to 800 mg/m² per dose, more preferably 100 to 600 mg/m² per dose, and even more preferably 250 to 400 mg/m² per dose. In another aspect, 5-FU is administered at approximately 1000 to 4000 mg/m² per dose, preferably 1500 to 3500 mg/m² per dose, more preferably 2400 to 3000 mg/m² per dose. In another aspect, 5-FU is administered at approximately 100 to 1000 mg/m² per dose, preferably 150 to 750 mg/m² per dose, more preferably 500 to 600 mg/m² per dose. In another aspect, 5-FU is administered at approximately 10 to 2000 mg/m² per dose, preferably 100 to 1500 mg/m² per dose, more preferably 200 to 1000 mg/m² per dose. In another aspect, 5-FU is administered at approximately 1000 to 3000 mg/m² per dose, preferably 1250 to 2500 mg/m² per dose, more preferably 1500 to 2400 mg/m² per dose.

When the AC is trifluridine, AC is administered at approximately 1 to 100 mg/m² per dose, preferably 10 to 50 mg/m² per dose, more preferably 20 to 40 mg/m² per dose, and even more preferably 25 to 35 mg/m² per dose.

When the AC is paclitaxel, AC is administered at approximately 10 to 500 mg/m² per dose, preferably 50 to 300 mg/m² per dose, more preferably 100 to 200 mg/m² per dose, and even more preferably 150 to 175 mg/m² per dose. In another aspect, paclitaxel is administered at approximately 10 to 500 mg/m² per dose, preferably 50 to 150 mg/m² per dose, more preferably 100 to 135 mg/m² per dose. In another aspect, paclitaxel is administered at approximately 10 to 300 mg/m² per dose, preferably 30 to 100 mg/m² per dose, more preferably 60 to 75 mg/m² per dose. In another aspect, paclitaxel is administered at approximately 50 to 500 mg/m² per dose, preferably 100 to 300 mg/m² per dose, more preferably 200 to 250 mg/m² per dose. In another aspect, paclitaxel is administered at approximately 10 to 500 mg/m² per dose, preferably 25 to 200 mg/m² per dose, more preferably 50 to 100 mg/m² per dose.

When the AC is oxaliplatin, AC is administered at approximately 1 to 200 mg/m² per dose, preferably 10 to 100 mg/m² per dose, more preferably 40 to 85 mg/m² per dose, and even more preferably 65 to 85 mg/m² per dose.

When the AC is cisplatin, AC is administered at approximately 1 to 100 mg/m² per dose, preferably 10 to 50 mg/m² per dose, more preferably 15 to 20 mg/m² per dose. In another aspect, cisplatin is administered at approximately 10 to 500 mg/m² per dose, preferably 50 to 300 mg/m² per dose, more preferably 75 to 100 mg/m² per dose. In another aspect, cisplatin is administered at approximately 10 to 300 mg/m² per dose, preferably 25 to 100 mg/m² per dose, more preferably 50 to 75 mg/m² per dose.

When the AC is bortezomib, AC is administered at approximately 0.1 to 10 mg/m² per dose, preferably 0.2 to 2 mg/m² per dose, more preferably 0.5 to 1.3 mg/m² per dose, and even more preferably 1.0 to 1.3 mg/m² per dose.

When the AC is compound 9, AC is administered orally in a range of doses, for example, 1 to 1500 mg per dose, for example, 2 to 800 mg per dose, for example, 5 to 500 mg per dose, for example, 2 to 200 mg per dose, or, for example, 10 to 100 mg.

When the compound represented by formula (I) or the pharmaceutically acceptable salt thereof, and at least one AC are separately formulated as two or more different preparations, the preparation containing the compound represented by formula (I) or the pharmaceutically acceptable salt thereof and at least one preparation containing an AC can be administered simultaneously, separately, or sequentially. The dosing interval for the separate administration is not particularly limited and can be selected so as to optimally exert the respective effects of the compound represented by formula (I) or the pharmaceutically acceptable salt thereof and at least one AC, and the effect of concomitant use. For the sequential administration, the preparation containing the compound represented by formula (I) or the pharmaceutically acceptable salt thereof and at least one preparation containing an AC can be administered in any order.

In the combination drug of the present invention, the preparation containing the compound represented by formula (I) or the pharmaceutically acceptable salt thereof and at least one preparation containing an AC may be administered through the same route or different routes. For example, both of the preparation containing the compound represented by formula (I) or the pharmaceutically acceptable salt thereof and at least one preparation containing an AC can be orally administered. Alternatively, for example, the preparation containing the compound represented by formula (I) or the pharmaceutically acceptable salt thereof can be orally administered while at least one preparation containing an AC can be administered by intravenous injection. The administration route can be appropriately determined according to the active ingredients to be administered and in consideration of the degree of progression of the malignant tumor in a patient, the general condition of the patient, etc.

The combination drug of the present invention can be administered to a patient before or after operation and can also be administered to an inoperable patient. The combination drug of the present invention can further contain a medicine for enhancing an antitumor effect and can also contain a medicine for reducing side effects.

In one aspect of the present invention, for the treatment of gastric cancer, for example, the compound represented by formula (I) or the pharmaceutically acceptable salt thereof and a pyrimidine antimetabolite tegafur/gimeracil/oteracil potassium can be concomitantly used, and both the medicines can be orally administered as active ingredients in the combination drug or as active ingredients in a pharmaceutical composition described below.

In another aspect of the present invention, for the treatment of gastric cancer, for example, the compound represented by formula (I) or the pharmaceutically acceptable salt thereof and an anti-microtubule agent paclitaxel can be concomitantly used, and the compound represented by formula (I) or the pharmaceutically acceptable salt thereof can be orally administered while paclitaxel can be intravenously administered.

In one aspect of the present invention, for the treatment of biliary tract cancer, for example, the compound represented by formula (I) or the pharmaceutically acceptable salt thereof and an anti-metabolite gemcitabine can be concomitantly used, and the compound represented by formula (I) or the pharmaceutically acceptable salt thereof can be orally administered while gemcitabine can be intravenously administered.

In another aspect of the present invention, for the treatment of biliary tract cancer, for example, the compound represented by formula (I) or the pharmaceutically acceptable salt thereof and a platinum-containing drug cisplatin can be concomitantly used, and the compound represented by formula (I) or the pharmaceutically acceptable salt thereof can be orally administered while cisplatin can be intravenously administered.

In one aspect of the present invention, for the treatment of bladder cancer, for example, the compound represented by formula (I) or the pharmaceutically acceptable salt thereof and a platinum-containing drug cisplatin can be concomitantly used, and the compound represented by formula (I) or the pharmaceutically acceptable salt thereof can be orally administered while cisplatin can be intravenously administered.

In one aspect of the present invention, for the treatment of brain tumor, for example, the compound represented by formula (I) or the pharmaceutically acceptable salt thereof and an alkylating agent temozolomide can be concomitantly used, and the compound represented by formula (I) or the pharmaceutically acceptable salt thereof can be orally administered while temozolomide can be intravenously or orally administered.

In one aspect of the present invention, for the treatment of endometrial cancer, for example, the compound represented by formula (I) or the pharmaceutically acceptable salt thereof and a platinum-containing drug cisplatin, an anti-metabolite gemcitabine, or a molecular targeting drug everolimus can be concomitantly used, and the administration route and dosing frequency of each medicine can be appropriately determined.

The administration or mixing ratios of Compound 1 or the pharmaceutically acceptable salt thereof and at least one AC are not particularly limited insofar as the ratios fall within a range that exerts an enhancing effect on an antitumor effect. Compound 1 or the pharmaceutically acceptable salt thereof can be used at approximately 0.1 to 7000 moles, preferably approximately 1 to 2000 moles, in terms of a free form per mole of at least one AC.

One of embodiments, the present invention also provides a pharmaceutical composition comprising the compound represented by formula (I) or a pharmaceutically acceptable salt thereof, and at least one AC.

The pharmaceutical composition of the present invention contains the compound represented by formula (I) or the pharmaceutically acceptable salt thereof, and at least one AC as active ingredients in the same composition, whereas the above-described combination drug comprises these active ingredients in separate preparations. The mixing ratios of the compound represented by formula (I) or the pharmaceutically acceptable salt thereof, and at least one AC in the composition may be within the range described above.

One of embodiments, the present invention also provides an antitumor effect enhancer for at least one AC, the antitumor effect enhancer comprising the compound represented by formula (I) or a pharmaceutically acceptable salt thereof as an active ingredient.

One of embodiments, the present invention also provides an antitumor agent comprising the compound represented by formula (I) or a pharmaceutically acceptable salt thereof, wherein the antitumor agent is concomitantly used with at least one AC.

One of embodiments, the present invention further provides a kit for malignant tumor treatment comprising the compound represented by formula (I) or a pharmaceutically acceptable salt thereof, and at least one AC.

One of embodiments, the present invention further provides an antitumor agent comprising the compound represented by formula (I) or a pharmaceutically acceptable salt thereof, wherein the antitumor agent is for the treatment of a cancer patient given at least one AC.

One of embodiments, the present invention further provides use of the compound represented by formula (I) or a pharmaceutically acceptable salt thereof in the manufacture of an antitumor effect enhancer for at least one AC.

One of embodiments, the present invention further provides use of the compound represented by formula (I) or a pharmaceutically acceptable salt thereof in the enhancement of the antitumor effect of at least one AC.

One of embodiments, the present invention further provides a method for treating a tumor comprising administering the compound represented by formula (I) or a pharmaceutically acceptable salt thereof, and at least one AC to a patient in need thereof.

One of embodiments, the present invention further provides a product containing as a first active ingredient a compound of formula (I), or a tautomer or a solvate or a pharmaceutically acceptable salt thereof, and as a further active ingredient at least one AC or a tautomer or a solvate or a pharmaceutically acceptable salt thereof, as a combined preparation for simultaneous, separate or sequential use in the treatment of patients suffering from cancer.

One of embodiments, the present invention further provides the use of a compound of formula (I), or a tautomer or a solvate or a pharmaceutically acceptable salt thereof, for treatment of a patient suffering from a cancer where the patient is being treated (or has been treated) with at least one AC, or a tautomer or a solvate or a pharmaceutically acceptable salt thereof.

One of embodiments, the present invention further provides a compound of formula (I), or a tautomer or a pharmaceutically acceptable salt or a solvate thereof, for use in combination therapy with at least one AC, to prevent, treat or manage cancer in a patient in need thereof.

One of embodiments, the present invention further provides a compound of formula (I), or a tautomer, or a pharmaceutically acceptable salt or a solvate thereof, for use in the prophylaxis or treatment of a disease state or condition as described herein, wherein the compound of formula (I) is used in combination with at least one AC, or a tautomer, or a pharmaceutically acceptable salt or a solvate thereof.

One of embodiments, the present invention further provides the use of the combination drug or a pharmaceutical composition comprising the combination drug for the manufacture of a medicament for use in the prophylaxis or treatment of a disease state or condition as described herein.

One of embodiments, the present invention further provides a combination drug wherein the compound of formula (I) and at least one AC are physically associated. In one embodiment the compound of formula (I) and at least one AC are: (a) in admixture; (b) chemically/physicochemically linked; (c) chemically/physicochemically co-packaged; or (d) unmixed but co-packaged or co-presented.

One of embodiments, the compound of formula (I) and at least one AC are non-physically associated. In another embodiment this optionally further includes (a) instructions for the extemporaneous association of the compound of formula (I) and at least one AC to form a physical association of the two or more compounds; or (b) instructions for combination therapy with the compound of formula (I) and at least one AC; or (c) instructions for administration to a patient population in which at least one AC have been (or are being) administered.

The embodiments of the present invention have been confirmed to be highly effective not only for a tumor having wild-type SHP2 but for a tumor having amplified or mutated SHP2. Accordingly, the target to be treated in the present invention also includes, but is not particularly limited to, tumors having wild-type SHP2, or amplified or mutated SHP2. Although the target to be treated in the present invention is not limited to a tumor having specific wild-type SHP2, it is preferably a tumor having wild-type SHP2. Furthermore, although the target to be treated in the present invention is not limited to a tumor having a specific SHP2 mutation, it is preferably a tumor having an SHP2 mutation.

The compound represented by formula (I) or the pharmaceutically acceptable salt thereof can be effectively used even for a tumor having resistance to any AC. Moreover, the compound represented by formula (I) or the pharmaceutically acceptable salt thereof can be effectively used even for a tumor having resistance to an SHP2 inhibitor other than the compound represented by formula (I) or the pharmaceutically acceptable salt thereof.

As known to persons skilled in the art, even medicines excellent in antitumor effect may inflict additional suffering to patients due to their side effects. The combination drug of the present invention can reduce the dose and dosing frequency of a medicine by the enhancement of the antitumor effect and can consequently be effective for the suppression of side effects.

### EXAMPLES

The present invention is explained in detail below with reference to Examples, however, the scope of the present invention is not limited to these Examples. Although the present invention is fully explained by means of Examples, it should be understood that persons skilled in the art can make various changes or modifications. Accordingly, such changes or modifications are encompassed in the present invention without departing from the scope of the present invention. Various reagents used in Examples were commercially available products unless otherwise specified.

### Synthetic Example of Compound 1 to 7

Compounds are named, for example, using an automated naming package such as AutoNom (MDL), using IUPAC rules or are as named by the chemical supplier. In the examples, the following abbreviations are used.
Ac: acetyl
aq.: aqueous
Boc: tert-butyloxycarbonyl
Boc2O: di-tert-butyl dicarbonate
BuLi: butyllithium
t-BuOK: potassium tert-butoxide
t-BuONa: sodium tert-butoxide
Cbz: carboxybenzyl
DBU: 1,8-diazabicyclo[5.4.0]undec-7-ene
DCM: dichloromethane
DIAD: diisopropyl azodicarboxylate
DIBAL-H: diisobutylaluminum hydride
DIPEA: N,N-diisopropylethylamine
DMA: N,N-dimethylacetamide
DME: 1,2-dimethoxyethane
DMEAD: di-2-methoxyethyl azodicarboxylate
DMF: N,N-dimethylformamide
DMSO: dimethyl sulfoxide
Et20: diethyl ether
EtOAc: ethyl acetate
EtOH: ethanol
h: hour
HATU: 0-(7-azabenzotriazol-1-yl)-N,N,N′,N′-tetramethyluronium hexafluorophosphate
HPLC: high performance liquid chromatography
IPA: isopropyl alcohol
LDA: lithium diisopropylamide
i-PrMgCl: isopropylmagnesium chloride
MeCN: acetonitrile
MeOH: methanol
min.: minutes
MS: mass spectrometry
MTBE: tert-butyl methyl ether
NaOMe: sodium methoxide
NBS: N-bromosuccinimde
NCS: N-chlorosuccinimide
NMP: N-methyl-2-pyrrolidinone
NMR: nuclear magnetic resonance spectroscopy
PMB: p-methoxybenzyl
PyBOP: (benzotriazol-1-yloxy)tripyrrolidinophosphonium
hexafluorophosphate
RT: room temperature
Sat.: saturated
SEM: (2-(trimethylsilyl)ethoxy)methyl
SEMCl: 2-(trimethylsilyl)ethoxymethyl chloride
TBAF: tetrabutylammonium fluoride
TBSCl: tert-butyldimethylsilyl chloride
TEA: triethylamine
TBME: tert-butyl methyl ether
TFA: trifluoroacetic acid
THF: tetrahydrofuran
TLC: thin layer chromatography
TsCl: p-toluenesulfonyl chloride
Z-chloride: benzyl chloroformate

### Synthetic Methods

All starting materials and solvents were obtained either from commercial sources or prepared according to the literature citation. Unless otherwise stated all reactions were stirred. Organic solutions were routinely dried over anhydrous magnesium sulfate. Hydrogenations were performed on a Parr hydrogenator, a Thales H-cube flow reactor under the conditions stated or under a balloon of hydrogen. Microwave reactions were performed in Biotage (Registered Trademark) Initiator, a CEM Discover and Smithcreator microwave reactor, heating to a constant temperature using variable power microwave irradiation. Normal phase column chromatography was routinely carried out on an automated flash chromatography system such as CombiFlash Companion or CombiFlash RF system using pre-packed silica (230-400 mesh, 40-63 μm) cartridges. SCX was purchased from Supelco and treated with 1M hydrochloric acid prior to use. Unless stated otherwise the reaction mixture to be purified was first diluted with MeOH and made acidic with a few drops of AcOH. This solution was loaded directly onto the SCX and washed with MeOH. The desired material was then eluted by washing with a solvent such as 1% NH3 in MeOH. NH₂ ion exchange silica gel purification was done with Strata NH₂ (55 μm, 70 Å) columns, loaded directly onto the NH₂ column and eluting with a solvent such as methanol. Biotage (Registered Trademark) SNAP Ultra silica gel columns and Biotage (Registered Trademark) KP-NH SNAP silica gel columns were purchased from Biotage (Registered Trademark). Reverse phase purification was done using Biotage (Registered Trademark) SNAP Ultra C18 silica gel columns and were purchased from Biotage (Registered Trademark).

### NMR Data

¹H-NMR spectra were acquired on a Bruker Avance III spectrometer at 400 MHz, an AL400 (400 MHz; produced by JEOL), a Mercury 400 (400 MHz; produced by Agilent Technologies, Inc.), a 500 MHz Bruker Avance III HD NMR Spectrometer or a Bruker Avance NEO NMR spectrometer (400 MHz). Either the central peaks of chloroform-d, dimethylsulfoxide-d₆ or an internal standard of tetramethylsilane were used as references. For NMR data, where the number of protons assigned is less than the theoretical number of protons in the molecule, it is assumed that the apparently missing signal(s) is/are obscured by solvent and/or water peaks. In addition, where spectra were obtained in protic NMR solvents, exchange of NH and/or OH protons with solvent occurs and hence such signals are normally not observed.

Analytical and Preparative LC-MS Systems

Analytical LC-MS System and Method Description

In the following examples, compounds were characterised by mass spectroscopy using the systems and operating conditions set out below. Where atoms with different isotopes are present and a single mass quoted, the mass quoted for the compound is the monoisotopic mass (i.e. 35Cl; 79Br etc.).

Shimadzu Nexera

HPLC System: Shimadzu SIL-30AC autosampler/2×Shimadzu LC-30AD pumps
Mass Spec Detector: Shimadzu LCMS-2020 single quadrupole MS
Second Detector: Shimadzu SPD-M20A diode array detector

### MS Operating Conditions

Qarray DC voltage: 20V on ES Pos (−20V on ES Neg)
Drying gas flow: 20.0 L/min

### DL Temperature: 300° C.

### Heat Block Temperature: 350° C.

### Nebulising Gas Flow: 1.5 L/min

### Scan Range: 100-750 amu

Ionisation Mode: ElectroSpray Positive-Negative switching
Agilent 1290 Infinity II—6130 LC-MS system

### HPLC System: Agilent 1290 Infinity II

Mass Spec Detector: Agilent 6130 single quadrupole

### Second Detector: Agilent 1290 Infinity II Diode Array

### Detector

### MS Operating Conditions

Capillary voltage: 3000V

### Fragmentor/Gain: 70

### Gain: 1

Drying gas flow: 13.0 L/min

### Gas Temperature: 350° C.

Nebuliser Pressure: 40 psig

### Scan Range: 150-1000 amu

### Sheath Gas Temperature: 360° C.

### Sheath Gas Flow: 10.0 L/min

Nozzle Voltage: 300 (+ve mode)/1750 (−ve mode)
Ionisation Mode: Agilent Jet Stream Electrospray Positive-Negative switching

LCMS spectra were alternatively measured with an SQD manufactured by Waters Corporation under the following two conditions, and the [M+H]⁺ values were shown.

MS detection: ESI positive

UV detection: 254 nm

Column flow rate: 0.5 mL/min

Mobile phase: water/acetonitrile (0.1% formic acid)

Injection volume: 1 μL

Method

Column: Acquity BEH, 2.1×50 mm, 1.7 μm

Gradient:

| Time | water/acetonitrile |
|---|---|
| (min) | (0.1% formic acid) |
| 0 | 95/5 |
| 0.1 | 95/5 |
| 2.1 | 5/95 |
| 3.0 | STOP |

Preparative LC-MS System and Method Description

Preparative LC-MS is a standard and effective method used for the purification of small organic molecules such as the compounds described herein. The methods for the liquid chromatography (LC) and mass spectrometry (MS) can be varied to provide better separation of the crude materials and improved detection of the samples by MS. Optimisation of the preparative gradient LC method will involve varying columns, volatile eluents and modifiers, and gradients. Methods are well known in the art for optimising preparative LC-MS methods and then using them to purify compounds. Such methods are described in Rosentreter U, Huber U.; Optimal fraction collecting in preparative LC-MS; J Comb Chem.; 2004; 6(2), 159-64 and Leister W, Strauss K, Wisnoski D, Zhao Z, Lindsley C., Development of a custom high-throughput preparative liquid chromatography/mass spectrometer platform for the preparative purification and analytical analysis of compound libraries; J Comb Chem.; 2003; 5(3); 322-9.

Several systems for purifying compounds via preparative LC-MS are described below although a person skilled in the art will appreciate that alternative systems and methods to those described could be used. From the information provided herein, or employing alternative chromatographic systems, a person skilled in the art could purify the compounds described herein by preparative LC-MS.

Mass Directed Purification LC-MS System

Preparative LC-MS is a standard and effective method used for the purification of small organic molecules such as the compounds described herein. The methods for the liquid chromatography (LC) and mass spectrometry (MS) can be varied to provide better separation of the crude materials and improved detection of the samples by MS. Optimisation of the preparative gradient LC method will involve varying columns, volatile eluents and modifiers, and gradients. Methods are well known in the art for optimising preparative LC-MS methods and then using them to purify compounds. Such methods are described in Rosentreter U, Huber U.; Optimal fraction collecting in preparative LC/MS; J Comb Chem.; 2004; 6(2), 159-64 and Leister W, Strauss K, Wisnoski D, Zhao Z, Lindsley C., Development of a custom high-throughput preparative liquid chromatography/mass spectrometer platform for the preparative purification and analytical analysis of compound libraries; J Comb Chem.; 2003; 5(3); 322-9.

One such system for purifying compounds via preparative LC-MS is described below although a person skilled in the art will appreciate that alternative systems and methods to those described could be used. In particular, normal phase preparative LC based methods might be used in place of the reverse phase methods described here. Most preparative LC-MS systems utilise reverse phase LC and volatile acidic modifiers, since the approach is very effective for the purification of small molecules and because the eluents are compatible with positive ion electrospray mass spectrometry. Employing other chromatographic solutions e.g. normal phase LC, alternatively buffered mobile phase, basic modifiers etc. as outlined in the analytical methods described above could alternatively be used to purify the compounds.

Agilent 1260 LC-MS preparative system

Hardware:

Autosampler: G2260A Prep ALS

Pumps: 2×G1361A Prep Pumps for preparative flow gradient,

G1311C Quat Pump VL for pumping modifier in prep flow and

G1310B Iso Pump for make-up pump flow

UV detector: G1365C 1260 MWD

MS detector: G6120B Quadrupole LC-MS

Fraction Collector: 2×G1364B 1260 FC-PS

G1968D Active Splitter

Software:

Agilent OpenLab C01.06

Agilent MS operating conditions:

Capillary voltage: 3000 V

Fragmentor/Gain: 70/1

Drying gas flow: 12.0 L/min

Drying Gas Temperature: 275° C.

Nebuliser Pressure: 40 psig

Vaporizer Temperature: 200° C.

Scan Range: 125-800 amu

Ionisation Mode: ElectroSpray Positive

Columns:

1. Waters XBridge Prep C18 5 m OBD 100×19 mm Typically used for ammonium bicarbonate-based methods

2. Waters SunFire Prep C18 OBD 5 m 100×19 mm Typically used for TFA-based methods

3. Waters XBridge Prep Phenyl 5 m OBD 100×19 mm Typically used for neutral pH ammonium acetate-based methods

4. Supelco Ascentis RP-Amide 5 m 100×21.2 mm Typically used for formic acid-based methods

5. Phenomenex Synergi Fusion-RP 4 m 100×21.2 mm Typically used for formic acid-based methods

Eluents:

Solvent A: Water

Solvent B: Acetonitrile

Solvent C: Choice of available modifiers:

2.5% Trifluoroacetic acid in water

2.5% Formic acid in water

250 mM ammonium bicarbonate in water pH 9.4

250 mM ammonium acetate

Make Up Solvent:

90:10 Methanol:Water+0.2% Formic Acid (for all chromatography types)

Methods:

According to the analytical trace the most appropriate preparative chromatography type was chosen. A typical routine was to run an analytical LC-MS using the type of chromatography (low or high pH) most suited for compound structure. Once the analytical trace showed good chromatography a suitable preparative method of the same type was chosen. Typical running conditions for both low and high pH chromatography methods were:

Flow rate: 25 mL/min

Gradient: Generally all gradients had an initial 0.4 min step with 95% A+5% B (with additional modifier C). Then according to analytical trace a 6.6 min gradient was chosen in order to achieve good separation (e.g. from 5% to 50% B for early retaining compounds; from 35% to 80% B for middle retaining compounds and so on)

Wash: 1.6 minute wash step was performed at the end of the gradient

Make Up flow rate: 0.8 mL/min

Solvent:

All compounds were usually dissolved in 100% MeOH or 100% DMSO From the information provided someone skilled in the art could purify the compounds described herein by preparative LC-MS.

Waters Fractionlynx system

Hardware:

2767 Dual Loop Autosampler/Fraction Collector

2525 preparative pump

CFO (column fluidic organiser) for column selection

RMA (Waters reagent manager) as make up pump

Waters ZQ Mass Spectrometer

Waters 2996 Photo Diode Array detector

Waters ZQ Mass Spectrometer

Software:

Masslynx 4.1

Waters MS running conditions:

Capillary voltage: 3.5 kV (3.2 kV on ES Negative)

Cone voltage: 25 V

Source Temperature: 120° C.

Multiplier: 500 V

Scan Range: 125-800 amu

Ionisation Mode: ElectroSpray Positive or ElectroSpray Negative

Alternatively Reverse phase preparative HPLC column chromatography was performed at the following conditions. Column: CAPCELL PAK C18 AQ manufactured by SHISEIDO, 30×50 mm, 5 μm

UV detection: 254 nm

Column flow rate: 40 mL/min

Mobile phase: water/acetonitrile (0.1% formic

acid)

Injection volume: 1.0 mL

Basic gradient method: water/acetonitrile 0%-50% (8 minutes)

Achiral Preparative Chromatography

The compound examples described have undergone HPLC purification, where indicated, using methods developed following recommendations as described in Snyder L. R., Dolan J. W., High-Performance Gradient Elution The Practical Application of the Linear-Solvent-Strength Model, Wiley, Hoboken, 2007.

Chiral Preparative Chromatography

Preparative separations using Chiral Stationary Phases (CSPs) are the natural technique to apply to the resolution of enantiomeric mixtures. Equally, it can be applied to the separation of diastereomers and achiral molecules. Methods are well known in the art for optimising preparative chiral separations on CSPs and then using them to purify compounds. Such methods are described in Beesley T. E., Scott R. P. W.; Chiral Chromatography; Wiley, Chichester, 1998.

### (1) Preparation 1: 2,4-Dichloro-5-iodo-7-((2-(trimethylsilyl)ethoxy)methyl)-7H-pyrrolo[2,3-d]pyrimidine

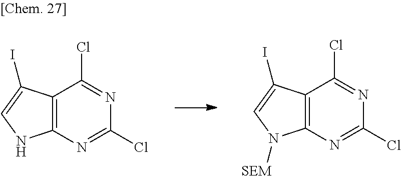 [SMILES: Clc1nc(Cl)c2c(I)c[nH]c2n1 ; C[Si](C)(C)CCOCn1cc(I)c2c(Cl)nc(Cl)nc21]

To a mixture of 2,4-dichloro-5-iodo-7H-pyrrolo[2,3-d]pyrimidine (1 g, 3.18 mmol) and DIPEA (1.66 mL, 9.55 mmol) in THF (10 mL) were added SEMCl (1.13 mL, 6.37 mmol) at RT. The mixture was stirred at RT for 2 h, and concentrated in vacuo. The residue was purified by column chromatography on silica gel (gradient elution, 0-30% EtOAc/hexane) to give the title compound (1.5 g). MS: [M+H]⁺=444, 446.

### (2) Preparation 2: 2-Chloro-5-iodo-7-((2-(trimethylsilyl) ethoxy)methyl)-3,7-dihydro-4H-pyrrolo[2,3-d]pyrimidin-4-one

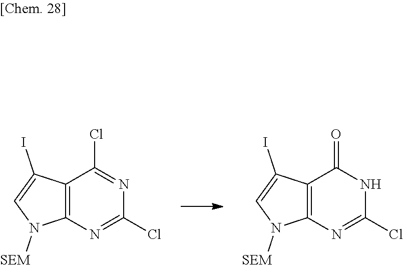 [SMILES: C[Si](C)(C)CCOCn1cc(I)c2c(Cl)nc(Cl)nc21 ; C[Si](C)(C)CCOCn1cc(I)c2c(=O)[nH]c(Cl)nc21]

To a mixture of 2,4-dichloro-5-iodo-7-((2-(trimethylsilyl) ethoxy)methyl)-7H-pyrrolo[2,3-d]pyrimidine (0.3 g, 0.67 mmol) in 1,4-dioxane (4 mL) was added 4 M KOH (1 mL, 4 mmol) at RT. The mixture was stirred at 60° C. overnight, cooled to RT, acidified with aq. HCl, and extracted with EtOAc. The organic layer was washed with brine, dried over anhydrous Na₂SO₄, filtered, and concentrated in vacuo. The residue was purified by column chromatography on silica gel (gradient elution, 0-70% EtOAc/hexane) to give the title compound (0.17 g). MS: [M+H]⁺=426, 428.

### (3) Preparation 3: 2-Chloro-5-iodo-3-methyl-7-((2-(trimethylsilyl) ethoxy)methyl)-3,7-dihydro-4H-pyrrolo[2,3-d]pyrimidin-4-one

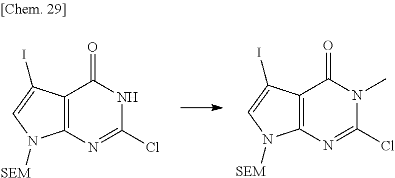 [SMILES: C[Si](C)(C)CCOCn1cc(I)c2c(=O)[nH]c(Cl)nc21 ; Cn1c(Cl)nc2c(c(I)cn2COCC[Si](C)(C)C)c1=O]

To a mixture of 2-chloro-5-iodo-7-((2-(trimethylsilyl) ethoxy)methyl)-3,7-dihydro-4H-pyrrolo[2,3-d]pyrimidin-4-one (0.17 g, 0.40 mmol) and K₂CO₃ (0.11 g, 0.79 mmol) in NMP (1 mL) was added iodomethane (0.05 mL, 0.79 mmol) at RT. The mixture was stirred at RT for 3 h, diluted with EtOAc, washed with water and brine, dried over anhydrous Na2SO4, filtered, and concentrated in vacuo. The residue was purified by column chromatography on silica gel (gradient elution, 0-50% EtOAc/hexane) to give the title compound (0.15 g). MS: [M+H]⁺=440, 442.

### (4) Preparation 4: 5-Bromo-4-chloro-2-methyl-2H-indazole

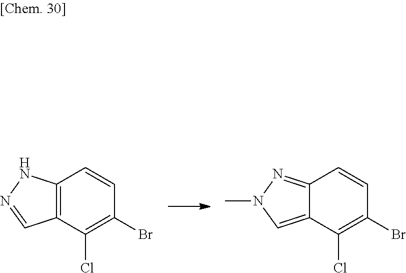

To a suspension of 5-bromo-4-chloro-1H-indazole (10.0 g, 43.2 mmol) in EtOAc (200 mL) was added trimethyloxonium tetrafluoroborate (9.58 g, 64.8 mmol) at RT. The mixture was stirred at RT for 20 h, quenched with sat. NaHCO₃, and extracted with EtOAc. The organic layer was washed with brine, dried over anhydrous Na₂SO₄, filtered, and concentrated in vacuo. The residue was purified by column chromatography on silica gel (gradient elution, 0-50% EtOAc/hexane) to give the title compound (9.16 g). MS: [M+H]⁺=245, 247.

### (5) Preparation 5: 5-Bromo-4-chloro-2-ethyl-2H-indazole

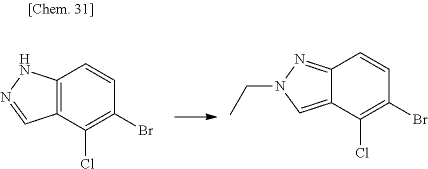

To a suspension of 5-bromo-4-chloro-1H-indazole (5.0 g, 21.6 mmol) in EtOAc (100 mL) was added triethyloxonium hexafluorophosphate (8.04 g, 32.4 mmol) at RT. The mixture was stirred at RT overnight, quenched with sat. NaHCO₃, and extracted with EtOAc. The organic layer was washed with brine, dried over anhydrous Na₂SO₄, filtered, and concentrated in vacuo. The residue was purified by column chromatography on silica gel (gradient elution, 0-50% EtOAc/hexane) to give the title compound (5.05 g). MS: [M+H]⁺=259, 261.

### (6) Preparation 6: 5-Bromo-4-fluoro-2-methyl-2H-indazole

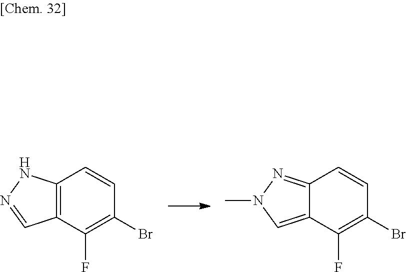

Prepared from 5-bromo-4-fluoro-1H-indazole using similar procedure for the preparation of 5-bromo-4-chloro-2-methyl-2H-indazole, to give the title compound. MS: [M+H]⁺=229, 231.

### (7) Preparation 7: 5-Bromo-3,4-dichloro-2-methyl-2H-indazole

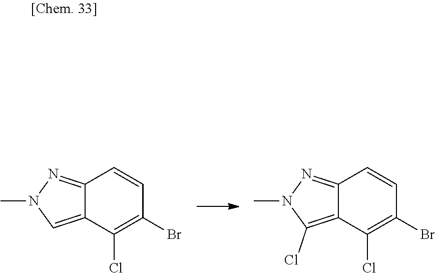

To a solution of 5-bromo-4-chloro-2-methyl-2H-indazole (5 g, 20.3 mmol) in DMF (50 mL) was added NCS (2.99 g, 22.4 mmol) at 0° C. The mixture was stirred at RT overnight. Water (150 mL) was added at RT. The mixture was stirred at RT for 1 h. The precipitate was collected, washed with water, and dried at 60° C. for 3 h under reduced pressure to give the title compound (5.63 g). MS: [M+H]⁺=279, 281.

### (8) Preparation 8: 5-Bromo-3-chloro-4-fluoro-2-methyl-2H-indazole

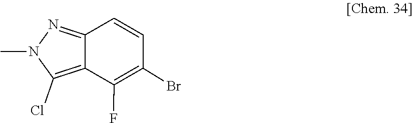

Prepared from 5-bromo-4-fluoro-2-methyl-2H-indazole using similar procedure for the preparation of 5-bromo-3,4-dichloro-2-methyl-2H-indazole, to give the title compound. MS: [M+H]⁺=263, 265.

### (9) Preparation 9: 4-Chloro-2-methyl-5-(4,4,5,5-tetramethyl-1,3,2-dioxaborolan-2-yl)-2H-indazole

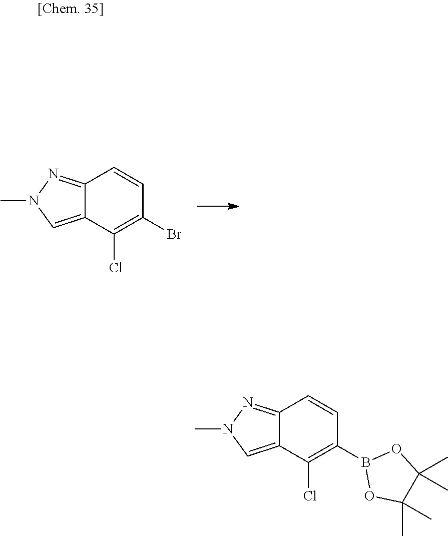

The mixture of 5-bromo-4-chloro-2-methyl-2H-indazole (12.14 g, 49.45 mmol), bis(pinacolato)diboron (18.83 g, 74.18 mmol), [1,1′-bis(diphenylphosphino)ferrocene]dichloropalladium(II) complex with dichloromethane (4.038 g, 4.945 mmol) and potassium acetate (9.706 g, 98.90 mmol) in 1,4-dioxane (120 mL) was degassed, purged with nitrogen, and stirred at 120° C. for 5 h. The reaction was cooled to RT, filtered through a pad of Celite, and washed with EtOAc. The filtrate was concentrated in vacuo. The residue was purified by column chromatography on NH silica gel (gradient elution, 0-70% EtOAc/hexane) to give the title compound (14.36 g). MS: [M+H]⁺=293, 295.

Compounds of table 1 below were prepared using procedures analogous to that described in preparation 9, starting from the appropriate substituted aryl halide (synthesized as described above with any significant variations indicated below).

TABLE 1

| | | MS : | |
|---|---|---|---|
| Compound | Compound name | [M+H]⁺ m/z | Procedure |
| 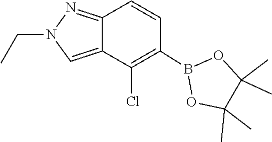 | 4-Chloro-2- ethyl-5- (4,4,5,5- tetramethyl- 1,3,2- dioxaborolan- 2-yl)-2H- indazole | 307, 309 | Prepared as preparation 9 above using 5- bromo-4- chloro-2- ethyl-2H- indazole |
| 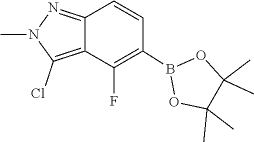 | 3-Chloro-4- fluoro-2- methyl-5- (4,4,5,5- tetramethyl- 1,3,2- dioxaborolan- 2-yl)-2H- indazole | 311, 313 | Prepared as preparation 9 above using 5- bromo-3- chloro-4- fluoro-2- methyl-2H- indazole |
| 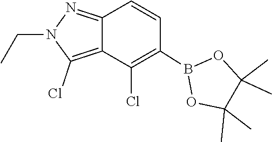 | 3,4-Dichloro- 2-methyl-5- (4,4,5,5- tetramethyl- 1,3,2- dioxaborolan- 2-yl)-2H- indazole | 327, 329 | Prepared as preparation 9 above using 5- bromo-3,4- dichloro-2- methyl-2H- indazole |

### (10) Preparation 10: tert-Butyl ((1R,2R,4S)-7-azabicyclo[2.2.11]heptan-2-yl) carbamate

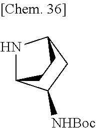 [SMILES: CC(C)(C)OC(=O)N[C@@H]1C[C@@H]2CC[C@H]1N2]

(Step 1)

rac-tert-Butyl ((1S,2S,4R)-7-azabicyclo[2.2.1]heptan-2-yl)carbamate hydrochloride (36 mg) was dissolved in DCM (2.89 mL). TEA (0.040 mL) and benzyl chloroformate (0.025 mL) were added thereto at RT, followed by stirring at RT for 1 h. The solvent was distilled off, and chloroform and water were added thereto. The mixture was extracted twice with chloroform and washed with water and saturated saline. The solvent was distilled off, and the residue was purified by silica gel column chromatography (gradient elution: hexane/EtOAc) to give benzyl rac-(1S,2S,4R)-2-((tert-butoxycarbonyl)amino)-7-azabicyclo[2.2.1]heptane-7-carboxylate. rac-Benzyl (1S,2S,4R)-2-((tert-butoxycarbonyl)amino)-7-azabicyclo[2.2.1]heptane-7-carboxylate was obtained as a 10 mg/mL ethanol solution, and separation was performed under the following conditions.

Column: Daicel CHIRALPAK IC 2.0×25 cm

Mobile phase: hexane/2-propanol=85/15

Flow rate: 12.5 mL/min

Retention time of each isomer:
Benzyl (1R,2R,4S)-2-((tert-butoxycarbonyl)amino)-7-azabicyclo[2.2.1]heptane-7-carboxylate: 16.93 minutes
Benzyl (1S,2S,4R)-2-((tert-butoxycarbonyl)amino)-7-azabicyclo[2.2.1]heptane-7-carboxylate: 23.82 minutes.

### Chiral Analysis Conditions:

Column: CHIRALPAK IC 4.6×150 mm

Mobile phase: hexane/2-propanol=85/15

Flow rate: 1.0 mL/min

Retention time of each isomer:
Benzyl (1R,2R,4S)-2-((tert-butoxycarbonyl)amino)-7-azabicyclo[2.2.1]heptane-7-carboxylate: 6.972 minutes
Benzyl (1S,2S,4R)-2-((tert-butoxycarbonyl)amino)-7-azabicyclo[2.2.1]heptane-7-carboxylate: 9.895 minutes.

(Step 2)

Benzyl (1R,2R,4S)-2-((tert-butoxycarbonyl)amino)-7-azabicyclo[2.2.1]heptane-7-carboxylate (93 g) and 10% Pd/C (10 g) were suspended in methanol (1.0 L). The mixture was stirred at RT for 5 h under a hydrogen atmosphere (50 psi). The reaction solution was filtrated, and the filtrate was concentrated to give the title compound. MS: [M+H]⁺=213. ¹H-NMR (DMSO-d₆) δ: 6.96-6.92 (1H, m), 3.63-3.56 (1H, m), 3.41-3.38 (1H, m), 3.35-3.32 (1H, m), 1.79-1.72 (1H, m), 1.67-1.61 (1H, m), 1.42-1.30 (11H, m), 1.27-1.19 (1H, m), 0.98-0.93 (1H, m).

### (11) Preparation 11: 2-chloro-5-(3,4-dichloro-2-methyl-2H-indazol-5-yl)-3-methyl-7-(2-trimethylsilylethoxymethyl)pyrrolo[2,3-d]pyrimidin-4-one

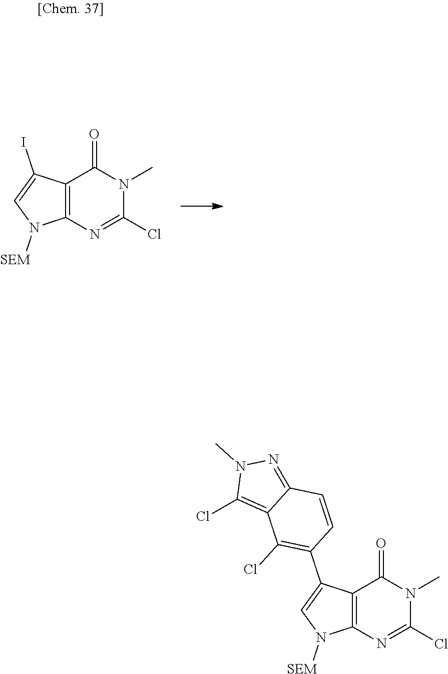 [SMILES: Cn1c(Cl)nc2c(c(I)cn2COCC[Si](C)(C)C)c1=O ; Cn1nc2ccc(-c3cn(COCC[Si](C)(C)C)c4nc(Cl)n(C)c(=O)c34)c(Cl)c2c1Cl]

The mixture of 2-chloro-5-iodo-3-methyl-7-(2-trimethylsilylethoxymethyl)pyrrolo[2,3-d]pyrimidin-4-one (107 mg, 0.244 mmol), 3,4-dichloro-2-methyl-5-(4,4,5,5-tetramethyl-1,3,2-dioxaborolan-2-yl)indazole (107 mg, 0.326 mmol), K₃PO₄ (108 mg, 0.506 mmol), bis(di-tert-butyl(4-dimethylaminophenyl)phosphine)dichloropalladium(II) (18 mg, 0.026 mmol), 1,4-dioxane (1.2 mL) and water (0.24 mL) was stirred at 70° C. for 5 h and cooled to RT. The mixture was purified by column chromatography on silica gel (gradient elution, 0-60% EtOAc/hexane) to give the title compound (113 mg). MS: [M+H]⁺=514, 516.

### (12) Preparation 12: tert-Butyl ((1R,2R,4S)-7-(5-(3,4-dichloro-2-methyl-2H-indazol-5-yl)-3-methyl-4-oxo-7-((2-(trimethylsilyl) ethoxy)methyl)-4,7-dihydro-3H-pyrrolo[2,3-d]pyrimidin-2-yl)-7-azabicyclo[2.2.1]heptan-2-yl)carbamate

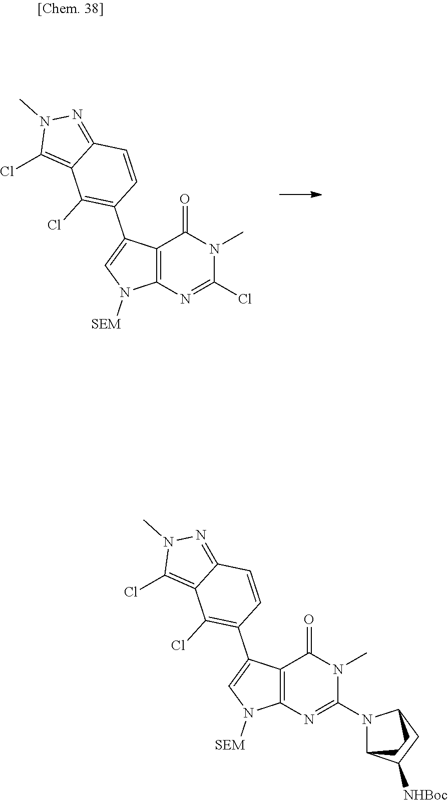 [SMILES: Cn1nc2ccc(-c3cn(COCC[Si](C)(C)C)c4nc(Cl)n(C)c(=O)c34)c(Cl)c2c1Cl ; Cn1nc2ccc(-c3cn(COCC[Si](C)(C)C)c4nc(N5[C@H]6CC[C@@H]5[C@H](NC(=O)OC(C)(C)C)C6)n(C)c(=O)c34)c(Cl)c2c1Cl]

The mixture of 2-chloro-5-(3,4-dichloro-2-methyl-2H-indazol-5-yl)-3-methyl-7-(2-trimethylsilylethoxymethyl)pyrrolo[2,3-d]pyrimidin-4-one (254 mg, 0.496 mmol), tert-butyl ((1R,2R,4S)-7-azabicyclo[2.2.1]heptan-2-yl)carbamate (161 mg, 0.760 mmol), DIPEA (0.847 mL, 4.95 mmol) and NMP (2.5 mL) was stirred at 120° C. for 22 h, cooled to RT, diluted with EtOAc, washed with water and brine, and concentrated in vacuo. The residue was purified by column chromatography on silica gel (gradient elution, 0-100% EtOAc/hexane) to give the title compound (340 mg). MS: [M+H]⁺=688, 690.

### (13) Preparation 13: 2-((1R,2R,4S)-2-amino-7-azabicyclo[2.2.1]heptan-7-yl)-5-(3,4-dichloro-2-methyl-2H-indazol-5-yl)-3-methyl-3,7-dihydro-4H-pyrrolo[2,3-d]pyrimidin-4-one (Compound 1)

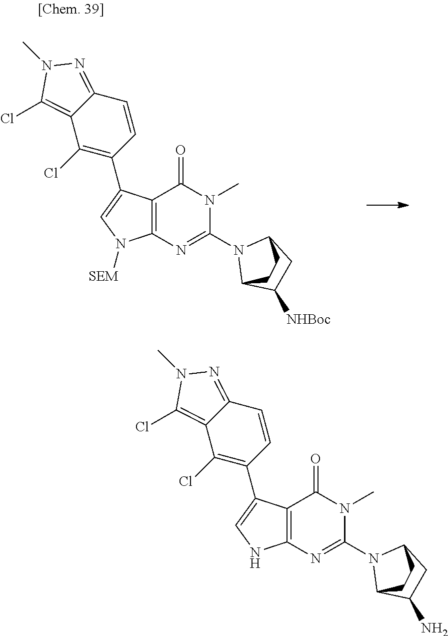 [SMILES: Cn1nc2ccc(-c3cn(COCC[Si](C)(C)C)c4nc(N5[C@H]6CC[C@@H]5[C@H](NC(=O)OC(C)(C)C)C6)n(C)c(=O)c34)c(Cl)c2c1Cl ; Cn1nc2ccc(-c3c[nH]c4nc(N5[C@H]6CC[C@@H]5[C@H](N)C6)n(C)c(=O)c34)c(Cl)c2c1Cl]

The mixture of tert-Butyl ((1R,2R,4S)-7-(5-(3,4-dichloro-2-methyl-2H-indazol-5-yl)-3-methyl-4-oxo-7-((2-(trimethylsilyl) ethoxy)methyl)-4,7-dihydro-3H-pyrrolo[2,3-d]pyrimidin-2-yl)-7-azabicyclo[2.2.1]heptan-2-yl)carbamate (793 mg, 1.15 mmol), trifluoroacetic acid (2 mL) and chloroform (4 mL) was stirred at 50° C. for 2 h, and concentrated in vacuo. The residue was purified by column chromatography on NH silica gel (gradient elution, 0-20% MeOH/CHCl₃). The obtained amorphous was dissolved in methanol (4 mL). To the solution was added ethylenediamine (0.385 mL, 5.77 mmol) at RT. The mixture was stirred at RT for 22 h, then water (4 mL) was added. The precipitate was collected, washed with water and ethyl acetate, and dried at 50° C. under reduced pressure to give the title compound (421 mg).

MS: [M+H]⁺=458, 460, ¹H-NMR (DMSO-d₆) δ: 11.67 (1H, br s), 7.50 (1H, d, J=8.8 Hz), 7.33 (1H, d, J=8.8 Hz), 6.97 (1H, s), 4.20-4.09 (4H, m), 4.01 (1H, br t, J=4.03 Hz), 3.54-3.44 (1H, m), 3.39 (3H, s), 2.33-2.21 (1H, m), 2.20-2.07 (1H, m), 1.98-1.84 (1H, m), 1.80-1.61 (1H, m), 1.56-1.41 (1H, m), 0.90 (1H, br dd, J=12.1, 4.0 Hz).

Compound 2 to 6 below were prepared using procedures analogous to that described in preparation 11-13 using the appropriate substituted indazoles, amines, pyrrolo[2,3-d]pyrimidin-4-one and 1,5-dihydro-4H-pyrazolo[3,4-d]pyrimidin-4-one

### (14) Compound 2: 6-((1R,4R,7R)-7-amino-2-azabicyclo[2.2.1]heptan-2-yl)-3-(3,4-dichloro-2-methyl-2H-indazol-5-yl)-5-methyl-1,5-dihydro-4H-pyrazolo[3,4-d]pyrimidin-4-one

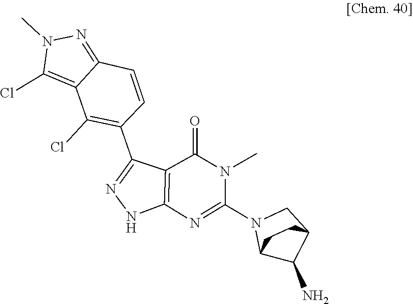 [SMILES: Cn1nc2ccc(-c3n[nH]c4nc(N5C[C@H]6CC[C@@H]5[C@@H]6N)n(C)c(=O)c34)c(Cl)c2c1Cl]

Prepared as preparation 11 to 13. In the last step, 4N HCl in dioxane and MeOH were used and then mixture was purified by NH silicagel (gradient elution, 0-10% MeOH/CHCl₃) to give title compound.

MS: [M+H]⁺=459, 461, ¹H-NMR (DMSO-d₆) δ: 13.20 (1H, br s), 7.60 (1H, d, J=8.8 Hz), 7.31 (1H, d, J=8.8 Hz), 4.16 (3H, s), 3.95-3.93 (1H, m), 3.79-3.75 (1H, m), 3.28 (3H, s), 3.26-3.24 (1H, m), 3.07 (1H, d, J=9.2 Hz), 2.19-2.14 (1H, m), 2.02-1.84 (3H, m), 1.40-1.33 (1H, m).

### (15) Compound 3: 2-((1R,4R,7R)-7-amino-2-azabicyclo[2.2.1]heptan-2-yl)-5-(4-chloro-2-ethyl-2H-indazol-5-yl)-3-methyl-3,7-dihydro-4H-pyrrolo[2,3-d]pyrimidin-4-one

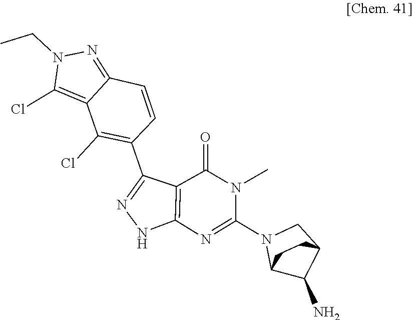 [SMILES: CCn1nc2ccc(-c3n[nH]c4nc(N5C[C@H]6CC[C@@H]5[C@@H]6N)n(C)c(=O)c34)c(Cl)c2c1Cl]

MS: [M+H]⁺=438, 440, ¹H-NMR (DMSO-d₆) δ: 11.49 (1H, br s), 8.42 (1H, s), 7.47 (1H, dd, J=9.0, 0.7 Hz), 7.30 (1H, d, J=9.0 Hz), 6.87 (1H, s), 4.46 (2H, q, J=7.3 Hz), 3.86-3.66 (2H, m), 3.30-3.26 (3H, m), 3.17-3.14 (1H, m), 3.04-2.98 (1H, m), 2.18-2.11 (1H, m), 2.01-1.82 (3H, m), 1.51 (3H, t, J=7.1 Hz), 1.40-1.31 (1H, m)

### (16) Compound 4: 2-((1R,4R,7R)-7-amino-2-azabicyclo[2.2.1]heptan-2-yl)-5-(4-chloro-2-methyl-2H-indazol-5-yl)-3-methyl-3,7-dihydro-4H-pyrrolo[2,3-d]pyrimidin-4-one

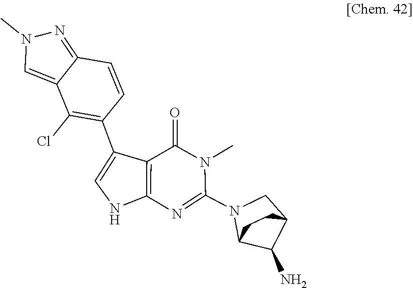 [SMILES: Cn1cc2c(Cl)c(-c3c[nH]c4nc(N5C[C@H]6CC[C@@H]5[C@@H]6N)n(C)c(=O)c34)ccc2n1]

MS: [M+H]⁺=424, 426, ¹H-NMR (DMSO-d₆) δ: 11.49 (1H, br s), 8.37 (1H, s), 7.46 (1H, d, J=8.9 Hz), 7.31 (1H, d, J=8.9 Hz), 6.88 (1H, s), 4.18 (3H, s), 3.84 (1H, s), 3.73-3.65 (1H, m), 3.28-3.23 (4H, m), 3.02 (1H, d, J=8.8 Hz), 2.15 (1H, s), 2.01-1.83 (3H, m), 1.74-1.49 (2H, m), 1.42-1.31 (1H, m)

### (17) Compound 5: 2-((1R,4R,7R)-7-amino-2-azabicyclo[2.2.1]heptan-2-yl)-5-(3,4-dichloro-2-methyl-2H-indazol-5-yl)-3-methyl-3,7-dihydro-4H-pyrrolo[2,3-d]pyrimidin-4-one

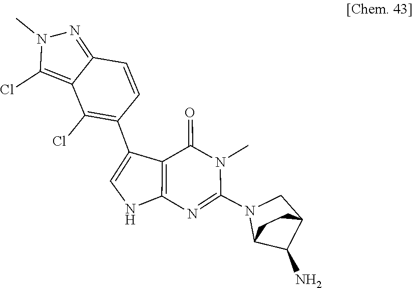 [SMILES: Cn1nc2ccc(-c3c[nH]c4nc(N5C[C@H]6CC[C@@H]5[C@@H]6N)n(C)c(=O)c34)c(Cl)c2c1Cl]

MS: [M+H]⁺=458, 460, ¹H-NMR (DMSO-d₆) δ: 11.51 (1H, br s), 7.49 (1H, d, J=9.0 Hz), 7.32 (1H, d, J=9.0 Hz), 6.90 (1H, s), 4.13 (3H, s), 3.85-3.83 (1H, m), 3.73-3.68 (1H, m), 3.28 (3H, s), 3.25-3.22 (1H, m), 3.01 (1H, d, J=9.0 Hz), 2.18-2.14 (1H, m), 2.00-1.84 (3H, m), 1.40-1.33 (1H, m).

### (18) Compound 6: 2-((1R,2R,4S)-2-amino-7-azabicyclo[2.2.1]heptan-7-yl)-5-(3-chloro-4-fluoro-2-methyl-2H-indazol-5-yl)-3-methyl-3,7-dihydro-4H-pyrrolo[2,3-d]pyrimidin-4-one

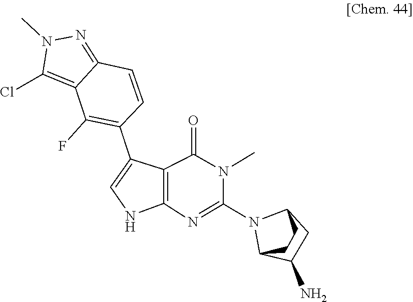 [SMILES: Cn1nc2ccc(-c3c[nH]c4nc(N5[C@H]6CC[C@@H]5[C@H](N)C6)n(C)c(=O)c34)c(F)c2c1Cl]

MS: [M+H]⁺=442, 444, ¹H-NMR (DMSO-d₆) δ: 11.67 (1H, br s), 7.66-7.60 (1H, m), 7.35 (1H, d, J=8.9 Hz), 7.02 (1H, s), 4.14-4.09 (4H, m), 4.01-3.97 (1H, m), 3.48-3.39 (4H, m), 2.33-2.10 (2H, m), 1.95-1.83 (2H, m), 1.74-1.60 (1H, m), 1.53-1.43 (1H, m), 0.91-0.80 (1H, m).

### (19) Compound 7: 2-((1R,2R,4S)-2-amino-7-azabicyclo[2.2.1]heptan-7-yl)-5-(4-chloro-2-ethyl-2H-indazol-5-yl)-3-methyl-3,7-dihydro-4H-pyrrolo[2,3-d]pyrimidin-4-one

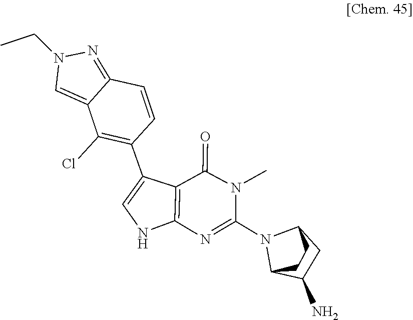 [SMILES: CCn1cc2c(Cl)c(-c3c[nH]c4nc(N5[C@H]6CC[C@@H]5[C@H](N)C6)n(C)c(=O)c34)ccc2n1]

MS: [M+H]⁺=438, 440. ¹H-NMR (DMSO-d₆) δ: 11.61 (1H, br s), 8.44 (1H, s), 7.49 (1H, d, J=8.8 Hz), 7.32 (1H, d, J=8.8 Hz), 6.93 (1H, s), 4.48 (2H, q, J=7.2 Hz), 4.14-4.12 (1H, m), 4.00-3.98 (1H, m), 3.48-3.43 (1H, m), 3.39 (3H, s), 2.30-2.22 (1H, m), 2.21-2.14 (1H, m), 1.96-1.88 (1H, m), 1.71-1.63 (2H, m), 1.54-1.46 (5H, m), 0.89-0.84 (1H, m).

### Example 1: SHP2 Biochemical Assay and Cellular pERK Inhibition Assay

### (1) SHP2 Biochemical Assay SHP2 Activity was Monitored by Measuring the Conversion of the Surrogate Substrate 6,8-Difluoromethylumbelliferyl Phosphate (DiEMUP) to the Fluorescent Product, 6,8-Difluoromethylumbelliferone (DiFMU).

SHP2 was pre-incubated with test compounds and the activating peptide pIRS1 (H₂N-LN(pY) IDLDLV-(PEG) 8-LST(pY)ASINFQK-amide) for 30 min, prior to addition of the 6,8-difluoromethylumbelliferyl phosphate (DiEMUP), (Thermo Fisher D6567). Final assay concentrations were 10 pM SHP2, 0.25 μM pIRS1 peptide, 50 μM DiFMUP, 25 mM Bis-Tris propane, pH 7.0, 150 mM NaCl, 0.05% (v/v) Tween-20, 0.5 mM TCEP and 5% (v/v) DMSO. Rates of reaction were then measured over 30 min by monitoring fluorescence on a BMG Pherastar reader at excitation 360 nm/emission 450 nm. IC₅₀ values were calculated in singlicate from the normalized dose-response plots using four parameter logistic curve fit. The Experiment for each compound was carried out in one time or multiple times, and the IC50 values were shown as a single value (for a compound measured in a single experiment) or an average value (for a compound measured in multiple experiments).

Results were as shown in the table 2.

(2) Cellular pERK Inhibition Assay

pERK levels were determined using the In-Cell Western assay. HCC827 cells (ATCC, Manassas, USA) were seeded into 384-well plates at a density of 1×10⁴ cells/well in RPMI1640 medium supplemented with 10% FBS and incubated 24 h. Compounds were diluted first in DMSO and then into serum-free medium, before being added to cells in quadruplicate to give a final concentration of 0.2% DMSO. Plates were incubated at 37° C. for the indicated time in a humidified atmosphere of 5% CO₂ in air.

Following compound treatment, cells were fixed with formalin neutral buffer solution for 20 minutes at room temperature. Plates were washed three times with 0.1% Triton-X in PBS and cells were blocked for 1 hour with Odyssey blocking buffer (LI-COR, #927-40000). After shaking out blocking buffer, cells were incubated with phospho-p44/42 ERK antibody (Cell Signaling Technology, #4370, 1:200) diluted in Odyssey blocking buffer at 4° C. overnight. Plates were washed again and cells were incubated for 1 hour with Goat anti-Rabbit IR Dye 800CW (LI-COR, #926-32211, 1:800) diluted in Odyssey blocking buffer. After washing and removing wash solution completely using a centrifuge machine, cells were scanned on the Odyssey (LI-COR), following the manufacturer's instructions. The average signal from blank wells (no cells added) was subtracted from the signals from each sample well. Levels of pERK were then expressed as percent of control, using DMSO treated samples as control. The relative IC50 values were calculated in quadruplicate from the normalized dose-response plots using four parameter logistic curve fit. The Experiment for each compound was carried out in one time or multiple times, and the IC₅₀ values were shown as a single value (for a compound measured in a single experiment) or an average value (for a compound measured in multiple experiments).

Results were shown in the table 2.

TABLE 2

| | SHP2 | pERK |
|---|---|---|
| Compound | (IC₅₀, μM) | (IC₅₀, μM) |
| 1 | 0.0020 | 0.021 |
| 2 | 0.0026 | 0.02 |
| 3 | 0.0040 | 0.035 |
| 4 | 0.0037 | 0.022 |
| 5 | 0.0015 | 0.016 |
| 6 | 0.0028 | 0.030 |
| 7 | 0.0033 | 0.032 |

### Example 2: Enhancement of Anti-Tumor Activity of EGFR Inhibitor

### (1) Anti-Proliferation Assay

Cell lines and culture medium was used as shown in table 3. Cell lines were obtained from ATCC or Health Science Research Resources Bank.

TABLE 3

| | | Cell number |
|---|---|---|
| Cell line | Culture medium | (Cells/20 μL/well) |
| NCI-H358, MKN45, | ATCC formulated | 500 |
| NCI-H23 | RPMI-1640 (10% | |
| | fetal bovine serum) | |
| RT 4 | McCoy's 5A (10% | 500 |
| | fetal bovine serum) | |
| MV-4-11 | IMDM (10% fetal | 500 |
| | bovine serum) | |
| RMG-I | Ham's F12 (10% | 500 |
| | fetal bovine serum) | |

384 well culture plate (781086, Greiner Bio-One International) was used for cell survival rate measurement assay. Each cell lines were collected by ordinary method, then suspended in indicated medium containing 10% fetal bovine serum in table 3. The number of cells seeded per well was set to 500 cells/20 μL. After incubation at 37° C. for 24 hours under 5% CO₂, Compound 1 and additional compound having an antitumor effect or a vehicle (DMSO) was added to each well by using D300e Digital Dispenser (Tecan). The concentration of Compound 1 was set to 10 concentrations. The concentration of each anti-cancer reagents set to 8 concentrations including 0 nM. After adding the medicine to the cells, the cells were further incubated at 37° C. for 3 days under 5% CO₂. Cell survival rates were calculated by adding 20 μL of CellTiter-Glo(registered trademark) 2. 0 (Promega) solution to each well, incubating the cells at room temperature for 10 minutes, and then measuring the chemiluminescence intensity of each well using a plate reader (ARVO).

A combination index (CI) value at each combined concentration of the medicines was determined. The combinatory effect of the two medicines was assessed as shown in table 4, (Trends Pharmacol. Sci. 4, 450-454, 1983; Pharmacol Rev. 2006, 58(3), 621-81).

TABLE 4

| CI Range (upper | |
|---|---|
| limit) | Description |
| <0.1 | Very strong synergy |
| 0.1-0.3 | Strong synergy |
| 0.3-0.7 | Synergy |
| 0.7-0.85 | Moderate synergy |
| 0.85-0.9 | Slight synergy |
| 0.9-1 | Almost additive |
| 1-1.2 | Slight antagonism |
| 1.2-1.45 | Moderate antagonism |
| 1.45-3.3 | Antagonism |
| 3.3-10 | Strong antagonism |
| >10 | Very strong antagonism |

### Example 3: Enhancement of Anti-Cancer Activity of Tyrosine Kinase Inhibitor

Anti-proliferation assay was conducted as described in Example 2. The compound 1 set ten concentrations and other anti-cancer medicines set 8 concentrations and the highest concentrations in each compounds were used as shown in table 5. CI value and combination effect also shown in table 6.

Result: Compound 1 synergistically enhanced anti-proliferation activity in combination with ALK inhibitor, Her family inhibitors (EGFR and HER2 inhibitors), BCR-ABL inhibitor, FLT3 inhibitor, multi-kinase inhibitor (PDGFR and VEGFR inhibitor), c-kit inhibitor.

TABLE 5

| | | Other anti-cancer |
|---|---|---|
| anti-cancer | Compound 1 (Highest | medicine(Highest |
| reagents | concentration, nM) | concentration, nM) |
| Erlotinib | 10000 | 10000 |
| Osimertinib | 10000 | 1000 |
| Getitinib | 10000 | 1000 |
| Anlotinib | 10000 | 10000 |
| Lapatinib | 10000 | 10000 |
| Neratinib | 10000 | 5000 |
| Afatinib | 10000 | 10000 |
| Sunitinib | 10000 | 10000 |
| Ponatinib | 10000 | 1000 |
| Nintedanib | 10000 | 10000 |
| Vandetanib | 10000 | 10000 |
| Brigatinib | 10000 | 3000 |
| Erdafitinib | 10000 | 10000 |
| Dasatinib | 10000 | 1000 |
| Gilteritinib | 10000 | 1000 |
| Alectinib | 10000 | 10000 |
| Crizotinib | 10000 | 10000 |

TABLE 6

| | | Molar ratio of | | Combination | | | |
|---|---|---|---|---|---|---|---|
| Combination | Cell | drugs Compound | Compound 1 | drug | | | |
| drug | line | 1:combination drug) | (nmol/L) | (nmol/L) | Fa | CI | Description |
| Erlotinib | NCI-H358 | 1:9 | 4.57 | 41.2 | 0.1600 | 0.617 | Strong |
| | | | 13.7 | 123 | 0.3804 | 0.145 | synergy |
| | | | 41.2 | 370 | 0.5542 | 0.102 | |
| | | | 123 | 1110 | 0.6026 | 0.206 | |
| | | | 370 | 3330 | 0.7034 | 0.256 | |
| | | | 1110 | 10000 | 0.8178 | 0.225 | |
| | RT4 | 1:81 | 4.57 | 370 | 0.2843 | 0.421 | Synergy |
| | | | 13.7 | 1110 | 0.4079 | 0.464 | |
| | | | 41.2 | 3330 | 0.5677 | 0.441 | |
| | | | 123 | 10000 | 0.7514 | 0.308 | |
| | MV-4-11 | 1:81 | 1.52 | 123 | 0.0915 | 0.808 | Synergy |
| | | | 4.57 | 370 | 0.2460 | 0.880 | |
| | | | 13.7 | 1110 | 0.6556 | 0.610 | |
| | | | 41.2 | 3330 | 0.8799 | 0.632 | |
| | | | 123 | 10000 | 0.9472 | 0.962 | |
| Osimertinib | NCI-H358 | 1:8.1 | 13.7 | 111 | 0.2157 | 0.333 | Synergy |
| | | | 41.2 | 333 | 0.4242 | 0.436 | |
| | | | 123 | 1000 | 0.5579 | 0.844 | |
| | RT4 | 1:8.1 | 4.57 | 37.0 | 0.2395 | 0.844 | Synergy |
| | | | 13.7 | 111 | 0.4891 | 0.453 | |
| | | | 41.2 | 333 | 0.7067 | 0.326 | |
| | | | 123 | 1000 | 0.7777 | 0.550 | |
| Gefitinib | NCI-H358 | 1:8.1 | 13.7 | 111 | 0.2078 | 0.311 | Synergy |
| | | | 41.2 | 333 | 0.4222 | 0.370 | |
| | | | 123 | 1000 | 0.5416 | 0.724 | |
| | RT4 | 1:8.1 | 13.7 | 111 | 0.5798 | 0.205 | Strong |
| | | | 41.2 | 333 | 0.6706 | 0.352 | synergy |
| | | | 123 | 1000 | 0.7956 | 0.445 | |
| Anlotinib | NCI-H358 | 1:9 | 41.2 | 370 | 0.3217 | 0.557 | Synergy |
| | | | 123 | 1110 | 0.5609 | 0.416 | |
| | | | 370 | 3330 | 0.7902 | 0.390 | |
| | | | 1110 | 10000 | 0.9031 | 0.488 | |
| | MKN45 | 1:27 | 123 | 3330 | 0.7514 | 0.755 | Moderate |
| | | | | | | | synergy |
| | RT4 | 1:9 | 13.7 | 123 | 0.3362 | 0.501 | Synergy |
| | | | 41.2 | 370 | 0.5003 | 0.444 | |
| | | | 123 | 1110 | 0.7040 | 0.387 | |
| | | | 370 | 3330 | 0.8765 | 0.326 | |
| | | | 1110 | 10000 | 0.9539 | 0.316 | |
| Lapatinib | MKN45 | 1:27 | 123 | 3330 | 0.6048 | 0.965 | Synergy |
| | | | 370 | 10000 | 0.9171 | 0.497 | |
| Neratinib | MKN45 | 1:4.5 | 123 | 556 | 0.5592 | 0.658 | Strong |
| | | | 370 | 1670 | 0.8104 | 0.336 | synergy |
| | | | 1110 | 5000 | 0.9240 | 0.237 | |
| Afatinib | NCI-H358 | 1:1 | 41.2 | 41.2 | 0.4605 | 0.143 | Strong |
| | | | 123 | 123 | 0.5395 | 0.264 | synergy |
| | | | 370 | 370 | 0.6382 | 0.426 | |
| | | | 1110 | 1110 | 0.7270 | 0.686 | |
| | | | 3330 | 3330 | 0.8696 | 0.526 | |
| | RT4 | 1:81 | 123 | 10000 | 0.9986 | 0.471 | Synergy |
| | NCI-H23 | 1:3 | 123 | 370 | 0.1325 | 0.776 | Synergy |
| | | | 370 | 1110 | 0.3961 | 0.397 | |
| | | | 1110 | 3330 | 0.8058 | 0.400 | |
| | | | 3330 | 10000 | 0.9563 | 0.471 | |
| | MV-4-11 | 1:27 | 13.7 | 370 | 0.6791 | 0.853 | Slight |
| | | | 41.2 | 1110 | 0.9244 | 0.906 | synergy |
| Sunitinib | NCI-H358 | 1:9 | 41.2 | 370 | 0.4562 | 0.502 | Synergy |
| | | | 123 | 1110 | 0.7002 | 0.747 | |
| | | | 370 | 3330 | 0.9541 | 0.612 | |
| | MKN45 | 1:81 | 123 | 10000 | 0.8052 | 0.818 | Moderate |
| | | | | | | | synergy |
| | RT4 | 1:81 | 4.57 | 370 | 0.1808 | 0.687 | Synergy |
| | | | 13.7 | 1110 | 0.4883 | 0.392 | |
| | | | 41.2 | 3330 | 0.6270 | 0.624 | |
| | MV-4-11 | 1:1 | 13.7 | 13.7 | 0.9463 | 0.262 | Strong |
| | | | 41.2 | 41.2 | 0.9651 | 0.327 | synergy |
| | | | 123 | 123 | 0.9666 | 0.902 | |
| Ponatinib | NCI-H358 | 1:2.7 | 41.2 | 111 | 0.2549 | 0.794 | Synergy |
| | | | 123 | 333 | 0.6294 | 0.463 | |
| | | | 370 | 1000 | 0.8028 | 0.639 | |
| | RT4 | 1:2.7 | 13.7 | 37.0 | 0.3693 | 0.454 | Synergy |
| | | | 41.2 | 111 | 0.6450 | 0.349 | |
| | | | 123 | 333 | 0.7489 | 0.602 | |
| | | | 370 | 1000 | 0.8589 | 0.842 | |
| Nintedanib | NCI-H358 | 1:27 | 4.57 | 123 | 0.0649 | 0.837 | Synergy |
| | | | 13.7 | 370 | 0.1639 | 0.576 | |
| | | | 41.2 | 1110 | 0.3485 | 0.580 | |
| | | | 370 | 10000 | 0.9756 | 0.858 | |
| | MKN45 | 1:81 | 41.2 | 3330 | 0.5044 | 0.925 | Synergy |
| | | | 123 | 10000 | 0.8817 | 0.418 | |
| | RMG-I | 1:9 | 13.7 | 123 | 0.2678 | 0.772 | Synergy |
| | | | 41.2 | 370 | 0.4576 | 0.477 | |
| | | | 123 | 1110 | 0.6821 | 0.683 | |
| | MV-4-11 | 1:3 | 13.7 | 41.2 | 0.7111 | 0.479 | Synergy |
| | | | 41.2 | 123 | 0.9278 | 0.370 | |
| | | | 123 | 370 | 0.9603 | 0.706 | |
| | | | 370 | 1110 | 0.9933 | 0.617 | |
| | | | 1110 | 3330 | 0.9982 | 0.770 | |
| Vandetanib | NCI-H358 | 1:9 | 41.2 | 370 | 0.2982 | 0.322 | Synergy |
| | | | 123 | 1110 | 0.5211 | 0.300 | |
| | | | 370 | 3330 | 0.6208 | 0.589 | |
| | | | 1110 | 10000 | 0.8142 | 0.733 | |
| | RT4 | 1:27 | 13.7 | 370 | 0.3118 | 0.486 | Synergy |
| | | | 41.2 | 1110 | 0.5143 | 0.444 | |
| | | | 123 | 3330 | 0.6645 | 0.570 | |
| | | | 370 | 10000 | 0.8261 | 0.541 | |
| | RMG-I | 1:9 | 41.2 | 370 | 0.4951 | 0.428 | Synergy |
| | | | 123 | 1110 | 0.5602 | 0.938 | |
| | MV-4-11 | 1:81 | 4.57 | 370 | 0.5454 | 0.912 | Strong |
| | | | 13.7 | 1110 | 0.8322 | 0.557 | synergy |
| | | | 41.2 | 3330 | 0.9448 | 0.503 | |
| | | | 123 | 10000 | 0.9916 | 0.294 | |
| Brigatinib | NCI-H358 | 1:2.7 | 41.2 | 111 | 0.3039 | 0.885 | Strong |
| | | | 123 | 333 | 0.6017 | 0.293 | synergy |
| | | | 370 | 1000 | 0.7788 | 0.506 | |
| | | | 1110 | 3000 | 0.9063 | 0.871 | |
| | RT4 | 1:24 | 4.57 | 111 | 0.2537 | 0.536 | Synergy |
| | | | 13.7 | 333 | 0.3969 | 0.558 | |
| | | | 41.2 | 1000 | 0.6106 | 0.459 | |
| | RMG-I | 1:8.1 | 13.7 | 111 | 0.2251 | 0.462 | Synergy |
| | | | 41.2 | 333 | 0.4905 | 0.493 | |
| | | | 123 | 1000 | 0.6133 | 0.963 | |
| | NCI-H23 | 1:2.7 | 41.2 | 111 | 0.0998 | 0.689 | Synergy |
| | | | 123 | 333 | 0.2711 | 0.353 | |
| | | | 370 | 1000 | 0.5739 | 0.447 | |
| | | | 1110 | 3000 | 0.8319 | 0.614 | |
| | MV-4-11 | 1:2.7 | 1.52 | 4.12 | 0.3537 | 0.844 | Strong |
| | | | 13.7 | 37.0 | 0.8161 | 0.430 | synergy |
| | | | 41.2 | 111 | 0.9418 | 0.286 | |
| | | | 123 | 333 | 0.9637 | 0.503 | |
| | | | 370 | 1000 | 0.9768 | 0.933 | |
| | | | 1110 | 3000 | 0.9918 | 0.966 | |
| Erdafitinib | RT4 | 1:0.1 | 13.7 | 1.37 | 0.3659 | 0.602 | Strong |
| | | | 41.2 | 4.12 | 0.6028 | 0.124 | synergy |
| | | | 123 | 12.3 | 0.6985 | 0.129 | |
| | NCI-H358 | 1:81 | 41.2 | 3330 | 0.3851 | 0.768 | Synergy |
| | | | 123 | 10000 | 0.7532 | 0.681 | |
| Dasatinib | NCI-H358 | 1:0.9 | 13.7 | 12.3 | 0.2486 | 0.342 | Strong |
| | | | 41.2 | 37.0 | 0.4351 | 0.208 | synergy |
| | | | 123 | 111 | 0.7042 | 0.119 | |
| | | | 370 | 333 | 0.7700 | 0.227 | |
| | | | 1110 | 1000 | 0.7795 | 0.633 | |
| | RT4 | 1:0.9 | 1.52 | 1.37 | 0.1983 | 0.804 | Strong |
| | | | 4.57 | 4.12 | 0.3708 | 0.356 | synergy |
| | | | 13.7 | 12.3 | 0.5917 | 0.147 | |
| | | | 41.2 | 37.0 | 0.6714 | 0.207 | |
| | | | 123 | 111 | 0.8271 | 0.096 | |
| | | | 370 | 333 | 0.8517 | 0.192 | |
| | | | 1110 | 1000 | 0.8974 | 0.229 | |
| Gilteritinib | NCI-H23 | 1:2.7 | 123 | 333 | 0.3005 | 0.540 | Synergy |
| | | | 370 | 1000 | 0.6542 | 0.666 | |
| | MV-4-11 | 1:0.3 | 4.57 | 1.37 | 0.5753 | 0.817 | Strong |
| | | | 13.7 | 4.12 | 0.8986 | 0.235 | synergy |
| | | | 41.2 | 12.3 | 0.9647 | 0.189 | |
| | | | 123 | 37.0 | 0.9763 | 0.354 | |
| | | | 370 | 111 | 0.9802 | 0.862 | |
| Alectinib | MKN45 | 1:27 | 41.2 | 1110 | 0.4310 | 0.905 | Synergy |
| | | | 370 | 10000 | 0.9801 | 0.542 | |
| | MV-4-11 | 1:27 | 4.57 | 123 | 0.5762 | 0.829 | Strong |
| | | | 13.7 | 370 | 0.8852 | 0.484 | synergy |
| | | | 41.2 | 1110 | 0.9534 | 0.609 | |
| | | | 123 | 3330 | 0.9967 | 0.187 | |
| | | | 370 | 10000 | 0.9962 | 0.631 | |
| Crizotinib | NCI-H23 | 1:3 | 1110 | 3330 | 0.5445 | 0.939 | Moderate |
| | | | 3330 | 10000 | 0.8672 | 0.765 | synergy |

### Example 4: Enhancement of Anti-Cancer Activity of RAS-MAPK Pathway Inhibitor

Anti-proliferation assay was conducted as described in Example 2. The compound 1 set ten concentrations and other anti-cancer medicines set 8 concentrations and the highest concentrations in each compounds were used as shown in table 7. CI value and combination effect also shown in table 8.

Result: Compound 1 synergistically enhanced anti-proliferation activity in combination with RAF inhibitor, MEK inhibitor or ERK inhibitor.

TABLE 7

| | | Other anti-cancer |
|---|---|---|
| anti-cancer | Compound 1 (Highest | medicine(Highest |
| reagents | concentration, nM) | concentration, nM) |
| Regoragfenib | 10000 | 10000 |
| Sorafenib | 10000 | 10000 |
| Trametinib | 1000 | 1000 |
| Cobimetinib | 10000 | 10000 |
| Binimetinib | 10000 | 10000 |
| Ulixertinib | 10000 | 10000 |

TABLE 8

| | | Molar ratio of | | Combination | | | |
|---|---|---|---|---|---|---|---|
| Combination | Cell | drugs Compound | Compound 1 | drug | | | |
| drug | line | 1:combination drug) | (nmol/L) | (nmol/L) | Fa | CI | Description |
| Regorafenib | RT4 | 1:9 | 13.7 | 123 | 0.1849 | 0.650 | Synergy |
| | | | 41.2 | 370 | 0.3071 | 0.582 | |
| | | | 123 | 1110 | 0.5417 | 0.359 | |
| | | | 370 | 3330 | 0.6157 | 0.696 | |
| | | | 1110 | 10000 | 0.7401 | 0.979 | |
| Sorafenib | MV-4-11 | 1:0.3 | 41.2 | 13.7 | 0.9439 | 0.116 | Strong |
| | | | 123 | 41.2 | 0.9578 | 0.158 | synergy |
| | | | 370 | 123 | 0.9643 | 0.301 | |
| | | | 1110 | 370 | 0.9665 | 0.759 | |
| | | | 3330 | 1110 | 0.9766 | 0.865 | |
| Trametinib | MKN45 | 1:0.1 | 37.0 | 4.12 | 0.4047 | 0.358 | Synergy |
| | | | 111 | 12.3 | 0.6614 | 0.389 | |
| | | | 333 | 37.0 | 0.7703 | 0.693 | |
| | RT4 | 1:0.3 | 4.12 | 1.37 | 0.1699 | 0.893 | Strong |
| | | | 12.3 | 4.12 | 0.3821 | 0.226 | synergy |
| | | | 37.0 | 12.3 | 0.4650 | 0.318 | |
| | | | 111 | 37.0 | 0.5406 | 0.487 | |
| | | | 333 | 111 | 0.6768 | 0.408 | |
| | | | 1000 | 333 | 0.7252 | 0.736 | |
| Cobimetinib | NCI-H358 | 1:0.3 | 123 | 41.2 | 0.3595 | 0.334 | Strong |
| | | | 370 | 123 | 0.5769 | 0.172 | synergy |
| | | | 1110 | 370 | 0.7439 | 0.165 | |
| | | | 3330 | 1110 | 0.8360 | 0.258 | |
| | | | 10000 | 3330 | 0.8699 | 0.585 | |
| | MKN45 | 1:9 | 13.7 | 123 | 0.2247 | 0.806 | Synergy |
| | | | 41.2 | 370 | 0.6457 | 0.459 | |
| | | | 123 | 1110 | 0.8262 | 0.616 | |
| Binimetinib | NCI-H358 | 1:9 | 4.57 | 41.2 | 0.0312 | 0.883 | Strong |
| | | | 13.7 | 123 | 0.1821 | 0.223 | synergy |
| | | | 41.2 | 370 | 0.3954 | 0.196 | |
| | | | 123 | 1110 | 0.6241 | 0.222 | |
| | | | 370 | 3330 | 0.7635 | 0.349 | |
| | | | 1110 | 10000 | 0.8387 | 0.673 | |
| | MKN45 | 1:9 | 13.7 | 123 | 0.2390 | 0.630 | Strong |
| | | | 41.2 | 370 | 0.5003 | 0.387 | synergy |
| | | | 123 | 1110 | 0.7765 | 0.212 | |
| | | | 370 | 3330 | 0.8408 | 0.360 | |
| Ulixertinib | NCI-H358 | 1:1 | 123 | 123 | 0.2823 | 0.709 | Strong |
| | | | 370 | 370 | 0.5072 | 0.379 | synergy |
| | | | 1110 | 1110 | 0.7490 | 0.237 | |
| | | | 3330 | 3330 | 0.8778 | 0.260 | |
| | | | 10000 | 10000 | 0.9536 | 0.284 | |
| | MKN45 | 1:3 | 123 | 370 | 0.5887 | 0.483 | Synergy |
| | | | 370 | 1110 | 0.7816 | 0.644 | |
| | RT4 | 1:9 | 13.7 | 123 | 0.1321 | 0.759 | Synergy |
| | | | 41.2 | 370 | 0.3352 | 0.310 | |
| | | | 123 | 1110 | 0.4921 | 0.330 | |
| | | | 370 | 3330 | 0.5670 | 0.622 | |

### Example 5: Enhancement of Anti-Cancer Activity of PI3K Pathway Inhibitor

Anti-proliferation assay was conducted as described in Example 2. The compound 1 set ten concentrations and other anti-cancer medicines set 8 concentrations and the highest concentrations in each compounds were used as shown in table 9. CI value and combination effect also shown in table 10.

Result: Compound 1 synergistically enhanced anti-proliferation activity in combination with PI3K inhibitor or AKT inhibitor.

TABLE 9

| | | Other anti-cancer |
|---|---|---|
| anti-cancer | Compound 1 (Highest | medicine(Highest |
| reagents | concentration, nM) | concentration, nM) |
| MK-2206 | 10000 | 10000 |
| Ideralisib | 10000 | 10000 |
| Alpelisib | 10000 | 10000 |

TABLE 10

| | | Molar ratio of | | Combination | | | |
|---|---|---|---|---|---|---|---|
| Combination | Cell | drugs Compound | Compound 1 | drug | | | |
| drug | line | 1:combination drug) | (nmol/L) | (nmol/L) | Fa | CI | Description |
| MK-2206 | NCI-H358 | 1:3 | 123 | 370 | 0.2532 | 0.477 | Strong |
| | | | 370 | 1110 | 0.4838 | 0.204 | synergy |
| | | | 1110 | 3330 | 0.5761 | 0.301 | |
| | | | 3330 | 10000 | 0.7446 | 0.213 | |
| | MKN45 | 1:27 | 1.52 | 41.2 | 0.1506 | 0.315 | Synergy |
| | | | 4.57 | 123 | 0.1981 | 0.564 | |
| | | | 13.7 | 370 | 0.3872 | 0.408 | |
| | | | 41.2 | 1110 | 0.5869 | 0.383 | |
| | | | 123 | 3330 | 0.7489 | 0.420 | |
| | | | 370 | 10000 | 0.8398 | 0.608 | |
| | RMG-I | 1:81 | 1.52 | 123 | 0.1204 | 0.724 | Synergy |
| | | | 4.57 | 370 | 0.2545 | 0.398 | |
| | | | 13.7 | 1110 | 0.4133 | 0.314 | |
| | | | 41.2 | 3330 | 0.5593 | 0.320 | |
| | | | 123 | 10000 | 0.6868 | 0.353 | |
| | NCI-H23 | 1:3 | 41.2 | 123 | 0.1015 | 0.783 | Strong |
| | | | 123 | 370 | 0.2110 | 0.265 | synergy |
| | | | 370 | 1110 | 0.3486 | 0.232 | |
| | | | 1110 | 3330 | 0.5074 | 0.295 | |
| | | | 3330 | 10000 | 0.7061 | 0.340 | |
| | MV-4-11 | 1:81 | 1.52 | 123 | 0.2639 | 0.315 | Strong |
| | | | 4.57 | 370 | 0.3899 | 0.520 | synergy |
| | | | 13.7 | 1110 | 0.6851 | 0.446 | |
| | | | 41.2 | 3330 | 0.9006 | 0.318 | |
| | | | 123 | 10000 | 0.9846 | 0.137 | |
| Idelalisib | RMG-I | 1:81 | 1.52 | 123 | 0.0902 | 0.778 | Synergy |
| | | | 13.7 | 1110 | 0.2747 | 0.511 | |
| | | | 41.2 | 3330 | 0.5012 | 0.333 | |
| | | | 123 | 10000 | 0.6059 | 0.570 | |
| | MV-4-11 | 1:81 | 1.52 | 123 | 0.2988 | 0.301 | Strong |
| | | | 4.57 | 370 | 0.3705 | 0.600 | synergy |
| | | | 13.7 | 1110 | 0.7136 | 0.330 | |
| | | | 41.2 | 3330 | 0.8924 | 0.262 | |
| | | | 123 | 10000 | 0.9566 | 0.273 | |
| Alpelisib | NCI-H358 | 1:9 | 41.2 | 370 | 0.1532 | 0.451 | Synergy |
| | | | 123 | 1110 | 0.4353 | 0.356 | |
| | | | 370 | 3330 | 0.5959 | 0.589 | |
| | | | 1110 | 10000 | 0.7671 | 0.844 | |
| | MKN45 | 1:27 | 13.7 | 370 | 0.1253 | 0.700 | Synergy |
| | | | 41.2 | 1110 | 0.4108 | 0.426 | |
| | | | 123 | 3330 | 0.5920 | 0.655 | |
| | | | 370 | 10000 | 0.8141 | 0.772 | |
| | RT4 | 1:81 | 1.52 | 123 | 0.0658 | 0.255 | Strong |
| | | | 13.7 | 1110 | 0.2690 | 0.247 | synergy |
| | | | 41.2 | 3330 | 0.4427 | 0.318 | |
| | | | 123 | 10000 | 0.5855 | 0.528 | |

### Example 6: Enhancement of Anti-Cancer Activity of BCL2 Inhibitor

Anti-proliferation assay was conducted as described in Example 2. The compound 1 set ten concentrations and other anti-cancer medicines set 8 concentrations and the highest concentrations in each compound was used as shown in table 11. CI value and combination effect also shown in table 12.

Result: Compound 1 synergistically enhanced anti-proliferation activity in combination with BCL2 inhibitor.

TABLE 11

| | | Other anti-cancer |
|---|---|---|
| anti-cancer | Compound 1 (Highest | medicine(Highest |
| reagents | concentration, nM) | concentration, nM) |
| Venetoclax | 10000 | 10000 |

TABLE 12

| | | Molar ratio of | | Combination | | | |
|---|---|---|---|---|---|---|---|
| Combination | Cell | drugs Compound | Compound 1 | drug | | | |
| drug | line | 1:combination drug) | (nmol/L) | (nmol/L) | Fa | CI | Description |
| Venetoclax | RT4 | 1:27 | 13.7 | 370 | 0.1751 | 0.479 | Synergy |
| | | | 41.2 | 1110 | 0.3605 | 0.497 | |
| | | | 123 | 3330 | 0.5099 | 0.888 | |
| | | | 370 | 10000 | 0.8781 | 0.888 | |
| | RMG-I | 1:9 | 123 | 1110 | 0.4133 | 0.687 | Synergy |
| | | | 370 | 3330 | 0.5921 | 0.601 | |
| | | | 1110 | 10000 | 0.9240 | 0.668 | |
| | MV-4-11 | 1:1 | 13.7 | 13.7 | 0.7436 | 0.424 | Very |
| | | | 41.2 | 41.2 | 0.9696 | 0.080 | strong |
| | | | 123 | 123 | 0.9987 | 0.007 | synergy |

### Example 7: Enhancement of Anti-Cancer Activity of CDK4/6 Inhibitor

Anti-proliferation assay was conducted as described in Example 2. The compound 1 set ten concentrations and other anti-cancer medicines set 8 concentrations and the highest concentrations in each compounds were used as shown in table 14.

Result: Compound 1 synergistically enhanced anti-proliferation activity in combination with CDK4/6 inhibitor.

TABLE 13

| | | Other anti-cancer |
|---|---|---|
| anti-cancer | Compound 1 (Highest | medicine(Highest |
| reagents | concentration, nM) | concentration, nM) |
| Palbociclib | 10000 | 10000 |
| Abemaciclib | 10000 | 10000 |

TABLE 14

| | | Molar ratio of | | Combination | | | |
|---|---|---|---|---|---|---|---|
| Combination | Cell | drugs Compound | Compound 1 | drug | | | |
| drug | line | 1:combination drug) | (nmol/L) | (nmol/L) | Fa | CI | Description |
| Palbociclib | NCI-H358 | 1:27 | 41.2 | 1110 | 0.1141 | 0.774 | Moderate |
| | | | 123 | 3330 | 0.2564 | 0.695 | synergy |
| | | | 370 | 10000 | 0.4277 | 0.833 | |
| | RMG-I | 1:81 | 13.7 | 1110 | 0.3192 | 0.411 | Strong |
| | | | 41.2 | 3330 | 0.4189 | 0.271 | synergy |
| | | | 123 | 10000 | 0.5120 | 0.225 | |
| | MV-4-11 | 1:81 | 1.52 | 123 | 0.1698 | 0.827 | Synergy |
| | | | 13.7 | 1110 | 0.6523 | 0.520 | |
| | | | 41.2 | 3330 | 0.7944 | 0.699 | |
| | | | 123 | 10000 | 0.9297 | 0.565 | |
| Abemaciclib | NCI-H358 | 1:3 | 13.7 | 41.2 | 0.1857 | 0.638 | Strong |
| | | | 41.2 | 123 | 0.3529 | 0.337 | synergy |
| | | | 123 | 370 | 0.5769 | 0.187 | |
| | | | 370 | 1110 | 0.7183 | 0.189 | |
| | | | 1110 | 3330 | 0.7441 | 0.455 | |
| | | | 3330 | 10000 | 0.9724 | 0.025 | |
| | MKN45 | 1:27 | 4.57 | 123 | 0.1096 | 0.665 | Synergy |
| | | | 41.2 | 1110 | 0.5496 | 0.647 | |
| | | | 123 | 3330 | 0.7785 | 0.698 | |
| | RMG-I | 1:27 | 1.52 | 41.2 | 0.1493 | 0.724 | Synergy |
| | | | 4.57 | 123 | 0.2479 | 0.606 | |
| | | | 13.7 | 370 | 0.4570 | 0.357 | |
| | | | 41.2 | 1110 | 0.5937 | 0.455 | |
| | | | 123 | 3330 | 0.6711 | 0.830 | |
| | NCI-H23 | 1:3 | 370 | 1110 | 0.3181 | 0.292 | Strong |
| | | | 1110 | 3330 | 0.5212 | 0.420 | synergy |
| | | | 3330 | 10000 | 0.9964 | 0.031 | |
| | MV-4-11 | 1:27 | 4.57 | 123 | 0.5416 | 0.592 | Synergy |
| | | | 13.7 | 370 | 0.8419 | 0.494 | |
| | | | 41.2 | 1110 | 0.9407 | 0.654 | |
| | | | 123 | 3330 | 0.9781 | 0.951 | |

### Example 8: Enhancement of Anti-Cancer Activity of HDAC Inhibitor

Anti-proliferation assay was conducted as described in Example 2. The compound 1 set ten concentrations and other anti-cancer medicines set 8 concentrations and the highest concentrations in each compounds were used as shown in table 15. CI value and combination effect also shown in table 16.

Result: Compound 1 synergistically enhanced anti-proliferation activity in combination with HDAC inhibitor.

TABLE 15

| | | Other anti-cancer |
|---|---|---|
| anti-cancer | Compound 1 (Highest | medicine(Highest |
| reagents | concentration, nM) | concentration, nM) |
| Vorinostat | 10000 | 10000 |

TABLE 16

| | | Molar ratio of | | Combination | | | |
|---|---|---|---|---|---|---|---|
| Combination | Cell | drugs Compound | Compound 1 | drug | | | |
| drug | line | 1:combination drug) | (nmol/L) | (nmol/L) | Fa | CI | Description |
| Vorinostat | NCI-H358 | 1:3 | 1110 | 3330 | 0.7678 | 0.642 | Synergy |
| | | | 3330 | 10000 | 0.9628 | 0.464 | |
| | RT4 | 1:27 | 1.52 | 41.2 | 0.0392 | 0.581 | Synergy |
| | | | 4.57 | 123 | 0.1081 | 0.388 | |
| | | | 13.7 | 370 | 0.1594 | 0.647 | |
| | | | 41.2 | 1110 | 0.4179 | 0.394 | |
| | | | 123 | 3330 | 0.5893 | 0.569 | |
| | | | 370 | 10000 | 0.8967 | 0.334 | |

### Example 9: Enhancement of Anti-Cancer Activity of Anti-Metabolite

Anti-proliferation assay was conducted as described in Example 2. The compound 1 set ten concentrations and other anti-cancer medicines set 8 concentrations and the highest concentrations in each compounds were used as shown in table 17. CI value and combination effect also shown in table 18.

Result: Compound 1 synergistically enhanced anti-proliferation activity in combination with anti-metabolite.

TABLE 17

| | | Other anti-cancer |
|---|---|---|
| | | medicine(Highest |
| anti-cancer | Compound 1 (Highest | concentration, |
| reagents | concentration, nM) | nM) |
| Gemcitabine | 1000 | 100 |
| 5-FU | 10000 | 10000 |
| FTD | 10000 | 10000 |

TABLE 18

| | | Molar ratio of | | Combination | | | |
|---|---|---|---|---|---|---|---|
| Combination | Cell | drugs Compound | Compound 1 | drug | | | |
| drug | line | 1:combination drug) | (nmol/L) | (nmol/L) | Fa | CI | Description |
| Gemcitabine | MKN45 | 1:0.1 | 12.3 | 1.23 | 0.2152 | 0.698 | Synergy |
| | | | 37.0 | 3.70 | 0.4083 | 0.878 | |
| 5-FU | MKN45 | 1:81 | 41.2 | 3330 | 0.4610 | 0.738 | Synergy |
| | | | 123 | 10000 | 0.7197 | 0.399 | |
| FTD | MKN45 | 1:27 | 123 | 3330 | 0.6654 | 0.716 | Synergy |
| | | | 370 | 10000 | 0.8785 | 0.494 | |
| | RT4 | 1:0.3 | 370 | 123 | 0.4667 | 0.468 | Synergy |
| | | | 1110 | 370 | 0.6009 | 0.676 | |
| | | | 3330 | 1110 | 0.7931 | 0.618 | |

### Example 10: Enhancement of Anti-Cancer Activity of Platinum-Containing Drug

Anti-proliferation assay was conducted as described in Example 2. The compound 1 set ten concentrations and other anti-cancer medicines set 8 concentrations and the highest concentrations in each compounds were used as shown in table 19. CI value and combination effect also shown in table 20.

Result: Compound 1 synergistically enhanced anti-proliferation activity in combination with platinum antitumor agent.

TABLE 19

| | | Other anti-cancer |
|---|---|---|
| anti-cancer | Compound 1 (Highest | medicine(Highest |
| reagents | concentration, nM) | concentration, nM) |
| Oxaliplatin | 10000 | 10000 |
| Cisplatin | 10000 | 10000 |

TABLE 20

| | | Molar ratio of | | Combination | | | |
|---|---|---|---|---|---|---|---|
| Combination | Cell | drugs Compound | Compound 1 | drug | | | |
| drug | line | 1:combination drug) | (nmol/L) | (nmol/L) | Fa | CI | Description |
| Oxalliplatin | NCI-H358 | 1:27 | 13.7 | 370 | 0.1376 | 0.699 | Synergy |
| | | | 41.2 | 1110 | 0.3478 | 0.424 | |
| | | | 123 | 3330 | 0.4795 | 0.623 | |
| | MKN45 | 1:27 | 13.7 | 370 | 0.4748 | 0.571 | Strong |
| | | | 41.2 | 1110 | 0.6458 | 0.322 | synergy |
| | | | 123 | 3330 | 0.7808 | 0.273 | |
| | | | 370 | 10000 | 0.9038 | 0.216 | |
| | RT4 | 1:9 | 4.57 | 41.2 | 0.2140 | 0.932 | Almost |
| | | | | | | | additive |
| Cisplatin | MKN45 | 1:27 | 41.2 | 1110 | 0.2057 | 0.694 | Synergy |
| | | | 123 | 3330 | 0.5148 | 0.626 | |
| | | | 370 | 10000 | 0.8006 | 0.681 | |
| | RT4 | 1:81 | 4.57 | 370 | 0.0886 | 0.632 | Synergy |
| | | | 13.7 | 1110 | 0.2585 | 0.539 | |

### Example 11: Enhancement of Anti-Cancer Activity of Anti-Microtubule Agent

Anti-proliferation assay was conducted as described in Example 2. The compound 1 set ten concentrations and other anti-cancer medicines set 8 concentrations and the highest concentrations in each compounds were used as shown in table 21. CI value and combination effect also shown in table 22.

Result: Compound 1 synergistically enhanced anti-proliferation activity in combination with microtubule inhibitor.

TABLE 21

| | | Other anti-cancer |
|---|---|---|
| anti-cancer | Compound 1 (Highest | medicine(Highest |
| reagents | concentration, nM) | concentration, nM) |
| Paclitaxel | 1000 | 100 |

TABLE 22

| | | Molar ratio of | | Combination | | | |
|---|---|---|---|---|---|---|---|
| Combination | Cell | drugs Compound | Compound 1 | drug | | | |
| drug | line | 1:combination drug) | (nmol/L) | (nmol/L) | Fa | CI | Description |
| Paclitaxel | MKN45 | 1:0.1 | 12.3 | 1.46 | 0.4372 | 0.106 | Strong |
| | | | 37.0 | 4.39 | 0.5083 | 0.222 | synergy |
| | | | 111 | 13.2 | 0.6577 | 0.307 | |
| | | | 333 | 39.5 | 0.7864 | 0.409 | |
| | | | 1000 | 119 | 0.8179 | 0.957 | |
| | RT4 | 1:0.03 | 4.12 | 0.14 | 0.1066 | 0.690 | Synergy |
| | | | 12.3 | 0.41 | 0.1742 | 0.828 | |
| | | | 37.0 | 1.23 | 0.4157 | 0.386 | |
| | | | 111 | 3.70 | 0.6114 | 0.366 | |
| | | | 333 | 11.1 | 0.7739 | 0.371 | |
| | | | 1000 | 33.3 | 0.8306 | 0.681 | |

### Example 12: Enhancement of Anti-Cancer Activity of Topoisomerase Inhibitor

Anti-proliferation assay was conducted as described in Example 2. The compound 1 set ten concentrations and other anti-cancer medicines set 8 concentrations and the highest concentrations in each compounds were used as shown in table 23. CI value and combination effect also shown in table 24.

Result: Compound 1 synergistically enhanced anti-proliferation activity in combination with topoisomerase inhibitor.

TABLE 23

| | | Other anti-cancer |
|---|---|---|
| anti-cancer | Compound 1 (Highest | medicine(Highest |
| reagents | concentration, nM) | concentration, nM) |
| SN-38 | 10000 | 500 |
| Etoposide | 10000 | 10000 |

TABLE 24

| | | Molar ratio of | | Combination | | | |
|---|---|---|---|---|---|---|---|
| Combination | Cell | drugs Compound | Compound 1 | drug | | | |
| drug | line | 1:combination drug) | (nmol/L) | (nmol/L) | Fa | CI | Description |
| SN-38 | MKN45 | 1:0.05 | 41.2 | 2.06 | 0.3061 | 0.750 | Strong |
| | | | 123 | 6.17 | 0.6496 | 0.390 | synergy |
| | | | 370 | 18.5 | 0.8647 | 0.261 | |
| | | | 1110 | 55.6 | 0.8972 | 0.536 | |
| | RT4 | 1:0.1 | 4.57 | 0.69 | 0.2202 | 0.322 | Very |
| | | | 13.7 | 2.06 | 0.2912 | 0.554 | strong |
| | | | 41.2 | 6.17 | 0.4442 | 0.621 | synergy |
| | | | 123 | 18.5 | 0.6700 | 0.472 | |
| | | | 370 | 55.6 | 0.7937 | 0.554 | |
| | | | 1110 | 167 | 0.9240 | 0.310 | |
| | | | 3330 | 500 | 0.9872 | 0.064 | |
| Etoposide | RT4 | 1:27 | 4.57 | 123 | 0.0637 | 0.460 | Synergy |
| | | | 13.7 | 370 | 0.2820 | 0.373 | |
| | | | 41.2 | 1110 | 0.5945 | 0.432 | |

### Example 13: Enhancement of Anti-Cancer Activity of Anthracycline Antibiotics

Anti-proliferation assay was conducted as described in Example 2. The compound 1 set ten concentrations and other anti-cancer medicines set 8 concentrations and the highest concentrations in each compound was used as shown in table 25. CI value and combination effect also shown in table 26.

Result: Compound 1 synergistically enhanced anti-proliferation activity in combination with anthracycline antibiotics.

TABLE 25

| | | Other anti-cancer |
|---|---|---|
| anti-cancer | Compound 1 (Highest | medicine(Highest |
| reagents | concentration, nM) | concentration, nM) |
| Doxorubicin | 10000 | 10000 |

TABLE 26

| | | Molar ratio of | | Combination | | | |
|---|---|---|---|---|---|---|---|
| Combination | Cell | drugs Compound | Compound 1 | drug | | | |
| drug | line | 1:combination drug) | (nmol/L) | (nmol/L) | Fa | CI | Description |
| Doxorubicin | NCI-H358 | 1:0.1 | 3330 | 370 | 0.7275 | 0.845 | Strong |
| | | | 10000 | 1110 | 0.9706 | 0.249 | synergy |
| | MKN45 | 1:1 | 123 | 123 | 0.5818 | 0.596 | Strong |
| | | | 370 | 370 | 0.8738 | 0.267 | synergy |
| | | | 1110 | 1110 | 0.9317 | 0.361 | |
| | RT4 | 1:0.3 | 41.2 | 13.7 | 0.2952 | 0.866 | Very |
| | | | 123 | 41.2 | 0.5056 | 0.403 | strong |
| | | | 370 | 123 | 0.6537 | 0.554 | synergy |
| | | | 3330 | 1110 | 0.9964 | 0.191 | |
| | | | 10000 | 3330 | 0.9999 | 0.063 | |

### Example 14: Measurement of Antitumor Effect of Concomitant Use of Compound 1 and Cetuximab on Tumor from Human Colorectal Cancer Cell Line SW837 Subcutaneously Implanted to SCID-Beige Mouse

With reference to Clin Cancer Invest. 2011, 121 (11): 4311-4321, a cell suspension of a human gastric cancer line SW837 (available from American Type Culture Collection) was subcutaneously implanted to 6-week-old male SCID-Beige mice (Charles River Japan, Inc.) at 8×10⁶ cells/mouse. For grouping (n=5/group), after the cell suspension implantation, tumor volumes (TV) were calculated according to the expression given below, and mice having TV of 100 to 200 mm³ were selected and assigned such that average TV was equal among groups. The day at which the grouping was carried out was defined as Day 0.

TV (mm³)=(Major axis×Minor axis²)/2 (units for the major axis and the minor axis were mm).

Compound 1 at 25 mg/kg/day was orally administered once a day for 28 days. And cetuximab at 1 mg/kg/day (in terms of erbitux) was administered intra-peritoneal injection on day 1, 4, 8, 11, 15, 18, 22, 25. The dose of Compound 1 was set to 25 mg/kg which corresponded to an effective dose for this mouse subcutaneous implantation model. The dose and dosing schedule has been successfully used in a number of xenograft models (Mol Cancer Ther 2006(5)104-113).

Antitumor effects were evaluated by using the difference between the average values of tumor volumes (TV) in two groups to be compared at the day of assessment, as an index. TV was calculated according to the expression given below from TV values on the day of measurement and on the day of grouping. Also, T/C (%) was calculated from the average RTV values in medicine administration groups and a control group.

RTV=(TV on the day of measurement)/(TV on the day of grouping)

T/C (%)=(Average RTV in each medicine administration group on the day of assessment)/(Average RTV in the control group on the day of assessment)×100

As a result, each of the treatment with Compound 1 (25 mg/kg) and the treatment with cetuximab (1 mg/kg) inhibited alone the growth of subcutaneously implanted SW837 tumor, with respective T/C (%) on the day of assessment being 65.0% and 37.7%. By contrast, the concomitant treatment with 25 mg/kg Compound 1 and 1 mg/kg cetuximab in combination inhibited tumor growth stronger than the treatment with each medicine alone, with respective T/C (%) being 29.3. The combination effect was significantly stronger than that of each monotherapy (P<0.05, Student's t test)

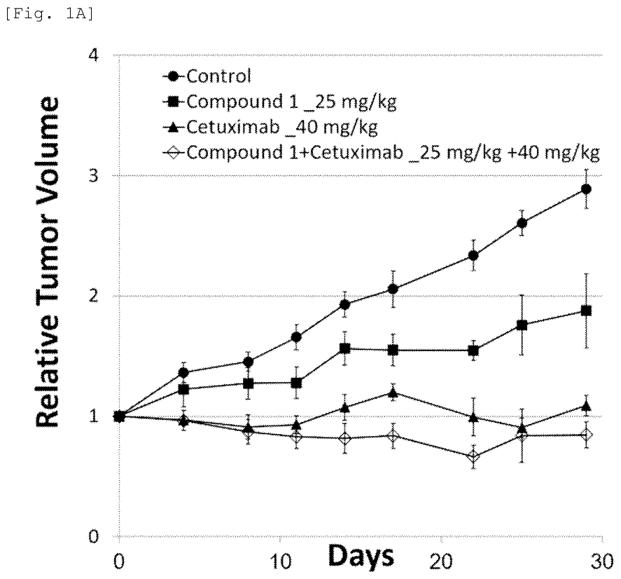

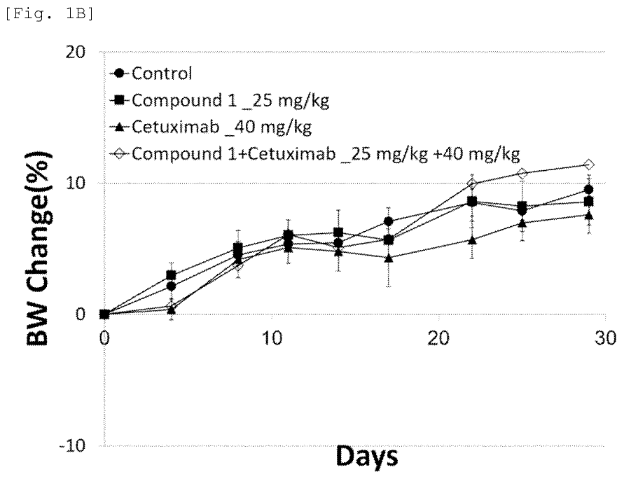

These results are shown in FIG. 1A. On the other hand, the average rate of body weight change in the Compound 1/cetuximab concomitant use group exhibited no significant difference from the rate of body weight change in each medicine group (FIG. 1B).

FIG. 1A shows the antitumor effects of Compound 1 and cetuximab used alone or concomitantly. The relative tumor volumes (RTV) in medicine administration groups and a control group are shown.

FIG. 1B shows the antitumor effects of Compound 1 and cetuximab used alone or concomitantly. The rates of mouse body weight change in medicine administration groups and a control group are shown.

### Example 15: Measurement of Antitumor Effect of Concomitant Use of Compound 1 and Compound 9 on Tumor from Human Pancreatic Cancer Cell Line, MIA PaCa-2, Subcutaneously Implanted to CB17 SCID Mouse

MIA PaCa-2 cell line carries KRAS mutation.

A human pancreatic cancer line, MIA PaCa-2 (available from American Type Culture Collection), suspended in a mixture of PBS and Matrigel was subcutaneously implanted to male CB17 SCID mice at 7-10 weeks of age (Charles River UK) at 5×10⁶ cells/100 μl/mouse. Tumors were measured with a pair of digital calipers, and tumor volumes (TV) were calculated by applying the formula for ellipsoid. Mice with TV of 146 to 360 mm³ were assigned to study groups such that average TV was equal among groups.

Oral administration of Compound 1 or its vehicle, and Compound 9 or its vehicle was started on Day 1. Compound 1 was dissolved in an acidified solution of 0.5% (w/v) hydroxypropyl methylcellulose (Sigma) and administered via oral gavage at 6 or 12 mg/kg/day. Compound 9 was suspended in 20% (v/v) PEG200 (Sigma) and 0.5% (w/v) methylcellulose (Sigma) and administered via oral gavage at 50 mg/kg/day. All treatments were given once a day for 21 consecutive days, except for the combination group receiving 12 mg/kg of Compound 1 and 50 mg/kg of Compound 9, in which the animals were treated for 5 days per week for 3 weeks.

Antitumor effects were evaluated by comparing the median relative tumor volumes (RTV) between control- and treated groups. RTV was calculated by dividing TV on any test day by TV on Day 1, expressed as a percentage. T/C (%), as a measure of anti-tumor activity, was calculated by dividing median RTV values of each compound-administered group by median RTV of the control group receiving both vehicles.

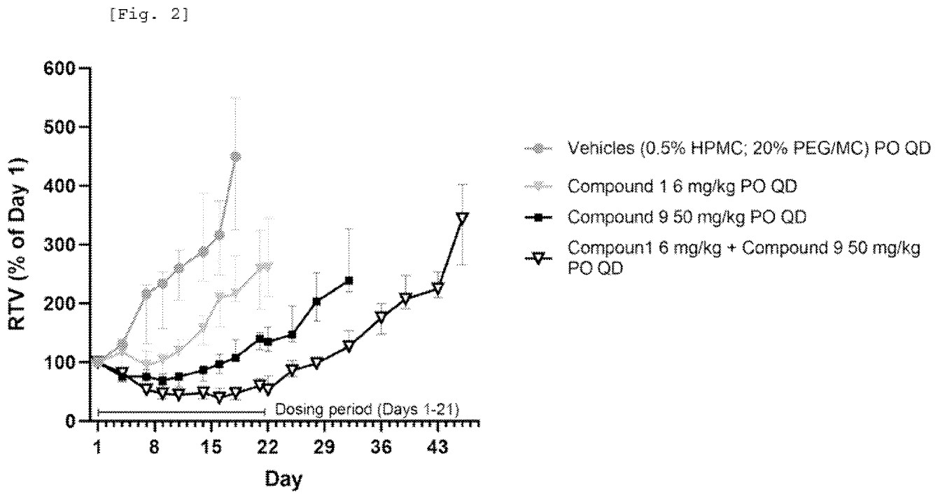

The tumor response to vehicles, Compound 1 at 6 mg/kg, Compound 9, and the combination of Compounds 1 and 9 is shown in FIG. 2. The treatment with Compound 1 (6 mg/kg) resulted in transient stasis and achieved a T/C of 58% on Day 18. The treatment with Compound 9 (50 mg/kg) resulted in tumor stasis during the treatment period, achieved T/C of 29% on Day 18 and partial regression (tumor shrinkage of 50% or above) in 1 out of 8 mice treated. The concomitant treatment with 6 mg/kg Compound 1 and 50 mg/kg Compound 9 in combination achieved partial regression in all 8 mice treated and a T/C of 13% on Day 18. The anti-tumor response was significantly stronger than that of either monotherapy (P<0.01 on Day 18, one-way ANOVA). No notable health issues were observed in this study.

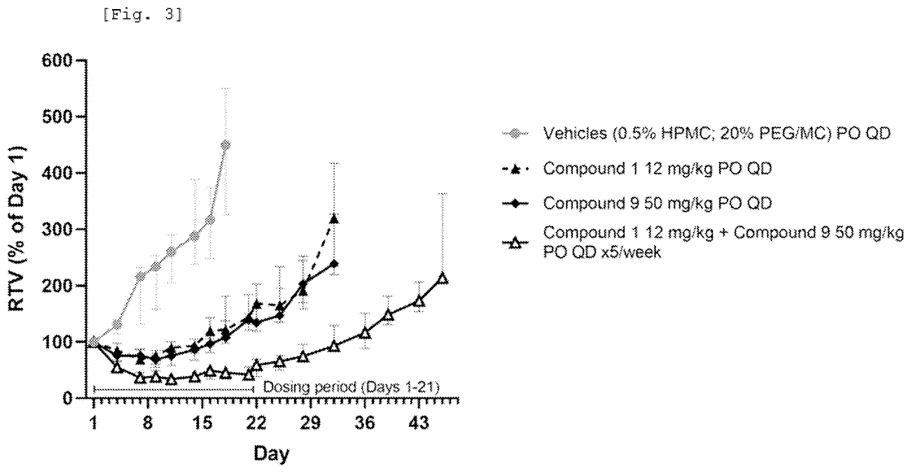

FIG. 3 shows the antitumor effects of the treatment combining Compound 1 at 12 mg/kg and Compound 9 at 50 mg/kg in an intermittent dose schedule and daily monotherapies. Treatment with Compound 1 at 12 mg/kg alone resulted in tumor stasis with a T/C of 33% on Day 18 and partial regression of the tumor in 2 out of 8 mice. This was similar to the treatment with Compound 9 alone (T/C of 29% and partial regression in 1 out of 8 mice). The combination treatment, given 5 days every week, resulted in partial regression in all 8 mice treated, and a T/C of 12% on Day 18. The anti-tumor response to the combination was greater than that of either monotherapies despite the reduced frequency of dosing (P<0.001 on Day 18, one-way ANOVA). No notable health issues were observed.

### Example 16: In Vitro Cell Line Screening for Combination of Compound 1 and Compound 9

Protocols

The effect of Compound 1 in combination with an ERK inhibitor, Compound 9, on 97 cell lines harboring driver mutations in KRAS (G12A, G12C, G12F, G12R, G12S, G12V, G13C, G13D, A59G, Q61H, Q61K, Q61L or A146T) was assessed using the following technique. Cells from human cancer cells lines (from commercial sources such as ATCC or ECCAC) were grown as 3D cultures by seeding cells into round-bottom ultra-low attachment 96-well tissue culture plates (Corning) followed by centrifugation. Cells were allowed to recover for 16-24 hours prior to compound treatment. Compounds or dimethyl sulfoxide (DMSO) were added at various combinations of compound concentrations in a final DMSO concentration of up to 0.5% (v/v). Following a total of 120-hour incubation, CellTiter-Glo 3D Reagent(registered trademark) (Promega) was added. Plates were incubated for 10 minutes with gentle shaking at room temperature to lyse the cells, then incubated for further 40 minutes at room temperature to stabilize luminescence signal. After mixing, the mixture was transferred to a fresh plate and luminescence was determined on EnVision plate reader (Perkin Elmer).

The effect of Compound 1 in combination with Compound 9 on 394 cell lines that do not depend on KRAS were tested using the following technique. Cells from human cancer cells lines (from commercial sources such as ATCC or ECCAC) were grown as 2D monolayers by seeding onto flat-bottom 96-well tissue culture plates (Corning). Cells were allowed to recover for 16-24 hours prior to compound treatment. Compounds or dimethyl sulfoxide (DMSO) were added at various combinations of compound concentrations in a final DMSO concentration of up to 0.5% (v/v). Following a total of 120-hour incubation, CellTiter-Glo reagent(registered trademark) (Promega) was added. Plates were incubated for 10 minutes with gentle shaking at room temperature to lyse the cells, then incubated for further 10 minutes at room temperature to stabilize luminescence signal. After mixing, the mixture was transferred to a fresh plate, and luminescence was determined on EnVision plate reader (Perkin Elmer).

For both 3D and 2D assays, the combination was tested as matrix of various concentrations of Compound 1 and Compound 9. The maximum concentrations of Compound 1 and Compound 9 used were 3 μM and 1 μM, respectively. Signals were normalized to that of the average of the DMSO control wells. Cell lines exhibiting maximum inhibition of greater than 50% relative to DMSO control in any of the wells were classified as sensitive. For each cell line, the maximum % inhibition values were obtained for Compound 1 alone, Compound 9 alone and the combination of both compounds. The degree of drug interaction (i.e. synergy, additivity or antagonism) was quantified for each cell line by the Bliss independence model using the Synergyfinder R package (Ref: He L. et al. (2018) Methods for High-throughput Drug Combination Screening and Synergy Scoring. In: von Stechow L. (eds) Cancer Systems Biology. Methods in Molecular Biology, vol 1711. Humana Press, New York, N.Y.). For each cell line, a single score metric, the sum of synergy and antagonism across screened drug concentrations (SUM_SYN_ANT) (Ref: Di Veroli G. Y. et al (2016) Bioinformatics 32(18):2866-2868), was calculated. Synergy was defined as a score of 21 or above.

Results

Table 27 summarizes the overall results of the screening panels, reporting the number of cell lines that were sensitive (inhibition of 50% or above) to Compound 1, Compound 9 or the combination of both. The number of cell lines sensitive to the combination was greater than those sensitive to either Compound 1 or Compound 9 alone. This was observed in both KRAS-dependent and -independent cell panels.

Combination-sensitive cell lines were further analyzed for the types of interaction. Synergistic interaction was found in both cell panels. Overall, 41% of the 491 cell lines tested were found to be sensitive to the combination and demonstrated synergistic interaction.

TABLE 27

| | Total | | | | |
|---|---|---|---|---|---|
| | of cell | Number of cell lines sensitive | | | |
| Cell | lines | (% of total) | | | |
| panel | number | Compound 1 | | Compound 9 | Combination |
| Total | 491 | 175 | (35.6) | 284 (57.8) | 371 (75.6) |
| KRAS | 97 | 38 | (39.2) | 80 (82.5) | 91 (93.8) |
| Non-KRAS | 394 | 137 | (34.8) | 204 (51.8) | 280 (71.1) |

Cell lines originating from lung cancer, ovarian cancer, cervical cancer, uterine/endometrial cancer, lymphoma, leukemia, myeloma, breast cancer, skin cancer, gastric cancer, esophageal cancer, colon cancer, colorectal cancer, kidney cancer, liver cancer, bile duct cancer, urinary bladder cancer, soft-tissue (bone cancer and other sarcoma) cancer, head and neck cancer, prostate cancer, thyroid cancer and pancreatic cancer, were found to be sensitive to the combination.

### INDUSTRIAL APPLICABILITY

The present invention can remarkably enhance an antitumor effect as compared with the administration of a conventionally known antitumor agent alone, and is also effective for tumors having drug resistance, thus can greatly expand the possibility of chemotherapy for malignant tumors.

All publications, patents and patent applications cited herein are incorporated herein by reference in their entirety.

1-48. (canceled)

49. A combination comprising a compound represented by formula (I): 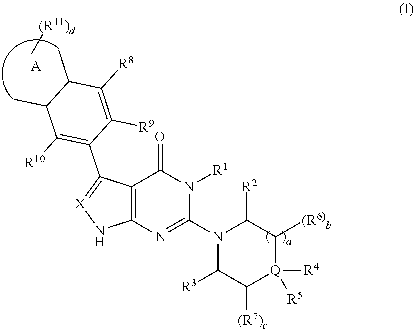
or a tautomer thereof, a solvate thereof, or a pharmaceutically acceptable salt thereof, wherein:
X is OH or N,
R¹ is —CH₃,
R² and R³ are independently selected from the group consisting of hydrogen and C₁₋₄alkyl,
Q is C or N,
wherein when Q is C, then either:
(i) R⁴ is amino, aminoC₁₋₄alkyl, or monoC₁₋₄alkylamino,
R⁵ is hydrogen, C₁₋₄alkyl, halogen, hydroxyC₁₋₄alkyl, C₁₋₄alkoxy, haloC₁₋₄alkyl, or C₁₋₄alkoxyC₁₋₄alkyl,
or
(ii) R⁴ and R⁵ together with Q form a four- to six-membered ring that can optionally contain 1 to 3 heteroatoms or groups independently selected from the group consisting of N, O, S, NH, C(O), and S(O)ₘ, and said ring formed by R⁴ and R⁵ can be unsubstituted or substituted with 1 to 4 groups independently selected from the group consisting of amino, halogen, haloC₁₋₄alkyl, hydroxyl, methoxy, methylamino, and C₁₋₄alkyl, and m is selected from the group consisting of 1 and 2, and
wherein when Q is N, then:
R⁴ is absent, and
R⁵ is hydrogen,
R⁶ and R⁷ are independently selected from the group consisting of halogen, C₁₋₄alkyl, hydroxyC₁₋₄alkyl, and hydroxyl, provided that when Q is N, then R⁶ or R⁷ is not halogen or hydroxyl,
or any two groups selected from the group consisting of R², R³, R⁶, and R⁷ together form a one- to three-membered bridge group selected from the group consisting of C₁₋₃alkylene, C₂₋₃alkenylene, methylene-NR^{q}-methylene, and methylene-O-methylene, wherein the bridge group is optionally substituted with a group selected from the group consisting of C₁₋₄alkyl, hydroxyl and halogen, and R^{q} is selected from the group consisting of hydrogen and C₁₋₄alkyl,
or R⁴ and R⁷ form a four- to six-membered ring containing a N atom,
or R⁵ and R⁷ form a three- to six-membered ring,
or R⁶ and R⁷ form a direct bond,
a is selected from the group consisting of 0, 1 and 2,
b is selected from the group consisting of 0, 1 and 2,
c is selected from the group consisting of 0, 1 and 2,
or Q is C, c is 2, R⁴ is hydrogen, and the two R⁷ join to form a 4 to 6-membered nitrogen containing ring,
Ring A is either:
(i) a five-membered nitrogen-containing heterocyclic ring wherein the heterocyclic ring optionally contains one or two additional heteroatoms selected from the group consisting of N, O, and S, or
(ii) a six-membered aromatic nitrogen-containing heterocyclic ring, wherein the heterocyclic ring optionally contains one or two additional heteroatoms selected from the group consisting of N, O, and S, or
(iii) a six-membered non-aromatic nitrogen-containing heterocyclic ring, wherein the heterocyclic ring optionally contains one or two additional heteroatoms selected from the group consisting of N and S,
R⁸ is selected from the group consisting of hydrogen, C₁₋₄alkyl, haloC₁₋₄alkyl, and halogen,
R⁹ is selected from the group consisting of hydrogen and halogen,
R¹⁰ is selected from the group consisting of haloC₁₋₄alkyl, C₁₋₄alkyl, halogen, hydrogen, and C₁₋₄alkoxy,
R¹¹ are independently selected from the group consisting of halogen, cyano, cyanoC₁₋₄alkyl, hydroxyl, oxo (═O), C₁₋₄alkyl optionally substituted with a five- or six-membered heterocyclic group containing 1 or 2 heteroatoms selected from the group consisting of O, N, and S, haloC₁₋₄alkyl, C₁₋₄alkoxy, hydroxylC₁₋₄alkyl, C₁₋₄alkoxyC₁₋₄alkyl, C₁₋₄alkylsulfone, amino, monoC₁₋₄alkylamino, diC₁₋₄alkylamino, aminoC₁₋₄alkyl, —C₁₋₄alkylene-C(═O)NH_{(2-q)}(C₁₋₆alkyl)_{q}), —C₁₋₄alkylene-NHC(═O)C₁₋₆alkyl, sulfonamide, sulfonamideC₁₋₄alkyl, 3 to 6-membered cycloalkyl, C₁₋₄alkyl substituted with 3 to 6-membered cycloalkyl, a five- or six-membered unsaturated heterocyclic group containing 1, 2, 3 or 4 heteroatoms selected from the group consisting of O, N, and S, and an optionally substituted four- to six-membered saturated heterocyclic group containing 1 or 2 heteroatoms selected from the group consisting of O, N, and S where the optional substituent is selected from C₁₋₄alkyl,
q is selected from the group consisting of 0, 1, and 2, and
d is selected from the group consisting of 0, 1, and 2, and
at least one additional compound having an antitumor effect, at least one tautomer thereof, at least one solvate thereof, or at least one pharmaceutically acceptable salt thereof, selected from the group consisting of molecular targeted drugs and cytotoxic drugs.

50. The combination drug according to claim 49, wherein the compound represented by formula (I) is selected from the group consisting of 2-((1R,2R,4S)-2-amino-7-azabicyclo[2.2.1]heptan-7-yl)-5-(3,4-dichloro-2-methyl-2H-indazol-5-yl)-3-methyl-3,7-dihydro-4H-pyrrolo[2,3-d]pyrimidin-4-one, 6-((1R,4R,7R)-7-amino-2-azabicyclo[2.2.1]heptan-2-yl)-3-(3,4-dichloro-2-methyl-2H-indazol-5-yl)-5-methyl-1,5-dihydro-4H-pyrazolo[3,4-d]pyrimidin-4-one, 2-((1R,4R,7R)-7-amino-2-azabicyclo[2.2.1]heptan-2-yl)-5-(4-chloro-2-ethyl-2H-indazol-5-yl)-3-methyl-3,7-dihydro-4H-pyrrolo[2,3-d]pyrimidin-4-one, 2-((1R,4R,7R)-7-amino-2-azabicyclo[2.2.1]heptan-2-yl)-5-(4-chloro-2-methyl-2H-indazol-5-yl)-3-methyl-3,7-dihydro-4H-pyrrolo[2,3-d]pyrimidin-4-one, 2-((1R,4R,7R)-7-amino-2-azabicyclo[2.2.1]heptan-2-yl)-5-(3,4-dichloro-2-methyl-2H-indazol-5-yl)-3-methyl-3,7-dihydro-4H-pyrrolo[2,3-d]pyrimidin-4-one, 2-((1R,2R,4S)-2-amino-7-azabicyclo[2.2.1]heptan-7-yl)-5-(3-chloro-4-fluoro-2-methyl-2H-indazol-5-yl)-3-methyl-3,7-dihydro-4H-pyrrolo[2,3-d]pyrimidin-4-one, and 2-((1R,2R,4S)-2-amino-7-azabicyclo[2.2.1]heptan-7-yl)-5-(4-chloro-2-ethyl-2H-indazol-5-yl)-3-methyl-3,7-dihydro-4H-pyrrolo[2,3-d]pyrimidin-4-one.

51. The combination drug according to claim 49, wherein the compound represented by formula (I) is 2-((1R,2R,4S)-2-amino-7-azabicyclo[2.2.1]heptan-7-yl)-5-(3,4-dichloro-2-methyl-2H-indazol-5-yl)-3-methyl-3,7-dihydro-4H-pyrrolo[2,3-d]pyrimidin-4-one.

52. The combination drug according to claim 49, wherein the at least one additional compound having an antitumor effect, the at least one tautomer thereof, the at least one solvate thereof, or the at least one pharmaceutically acceptable salt thereof is selected from the group consisting of a tyrosine kinase inhibitor, a RAS-MAPK pathway inhibitor, a PI3K pathway inhibitor, a BCL2 inhibitor, a CDK4/6 inhibitor, an HDAC inhibitor, a topoisomerase inhibitor (a topoisomerase I inhibitor, and topoisomerase II inhibitor), an alkylating agent, an anthracycline antibiotic, an alkaloid, an anti-metabolite, an anti-microtubule agent, a platinum-containing drug, a proteasome inhibitor, and a thalidomide analog drug.

53. The combination drug according to claim 49, wherein the molecular targeted drug is selected from the group consisting of an AKT inhibitor, ALK inhibitor, Bcl2 inhibitor, BCR-ABL inhibitor, BRAF inhibitor, CDK4/6 inhibitor, CDK inhibitor, EGFR inhibitor, Erk1/2 inhibitor, FGFR inhibitor, FLT3 inhibitor, HER2 inhibitor, MEK inhibitor, Multi-kinase inhibitor, PI3K inhibitor, RAF inhibitor, HDAC inhibitor, topoisomerase I inhibitor, topoisomerase II inhibitor, alkylating agent, anthracycline antibiotics, alkaloid, anti-metabolite, anti-microtubule agent, platinum-containing drug, and proteasome inhibitor.

54. The combination drug according to claim 49, wherein the molecular targeted drug is selected from the group consisting of cetuximab, MK-2206, alectinib, crizotinib, venetoclax, imatinib, dasatinib, ponatinib, dabrafenib, vemurafenib, sorafenib, palbociclib, abemaciclib, osimertinib, gefitinib, erlotinib, afatinib, brigatinib, ulixertinib, (2R)-2-(6-{5-chloro-2-[(oxan-4-yl)amino]pyrimidin-4-yl}-1-oxo-2,3-dihydro-1H-isoindol-2-yl)-N-[(1S)-1-(3-fluoro-5-methoxyphenyl)-2-hydroxyethyl]propanamide or a salt thereof, erdafitinib, giltertinib, lapatinib, neratinib, trametinib, cobimetinib, binimetinib, regorafenib, sunitinib, nintedanib, anlotinib, vandetanib, lenvatinib, alpelisib and idelalisib/CAL-101, vorinostat (SAHA), irinotecan (SN-38), etoposide, cyclophosphamide, doxorubicin, gemcitabine, pemetrexed, 5-FU, FdUrd, FTD, paclitaxel, cisplatin, oxaliplatin, bortezomib, afuresertib, trans-3-amino-1-methyl-3-(4-(3-phenyl-5H-imidazo[1,2-c]pyrido[3,4-e][1,3]oxazin-2-yl)phenyl)cyclobutanol, capivasertib, ipatasertib, triciribine, miransertib, lorlatinib, ceritinib, repotrectinib, ensartinib, alkotinib, WX-0593, SAF-189s, CT-707, TQ-B3101, sabutoclax, apogossypol, obatoclax, navitoclax, APG-2575, APG-1252, asciminib, olverembatinib, encorafenib, lifirafenib, LXH-254, ribociclib, lerociclib, trilaciclib, alvocidib, GLR-2007, SHR-6390, XZP-3287, BPI-1178, PF-06873600, NUV-422, FCN-437, seliciclib, mevociclib, milciclib, fadraciclib, zotiraciclib, dinaciclib, samuraciclib, voruciclib, FIT-039, PF-07104091, BEY-1107, panitumumab, sutetinib, allitinib, epitinib, xiliertinib, rociletinib, dacomitinib, simotinib, olmutinib, yinlitinib, mefatinib, alflutinib, almonertinib, icotinib, naquotinib, poziotinib, epertinib, sapitinib, cipatinib, tarloxotinib, pyrotinib, pirotinib, lazertinib, varlitinib, tesevatinib, canertinib, mobocertinib, duligotuzumab, olafertinib, zorifertinib, pelitinib, DZD-9008, ASK-120067, BPI-7711, QLNC-120, ametumumab, imgatuzumab, amivantamab, seribantumab, nimotuzumab, serclutamab, depatuxizumab, tomuzotuximab, SCT-200, ravoxertinib, LY3214996, MK-8353, LTT462, HH-2710, infigratinib, pemigatinib, orantinib, derazantinib, roblitinib, rogaratinib, zoligratinib, E-7090, AZD-4547, ODM-203, ICP-192, HMPL-453, bemarituzumab, quizartinib, crenolanib, flysyn, mivavotinib, PHI-101, MEN-1703, FF-10101, HM-43239, E-6201, ENMD-2076, larotinib, tucatinib (irbinitinib), BDTX-189, trastuzumab, pertuzumab, zanidatamab, zenocutuzumab, margetuximab, KN-026, BAT-8001, TAA-013, KL-A166, selumetinib, refametinib, mirdametinib, pimasertib, HL-085, NFX-179, nilotinib, dovitinib, axitinib, vatalinib, pazopanib, avapritinib, famitinib, catequentinib, necuparanib, surufatinib, lucitanib, midostaurin, vorolanib, bevasiranib, bevacizumab, ranibizumab, vanucizumab, navicixizumab, ramucirumab, BAT-5906, VGX-100, CSL-346, duvelisib, copanlisib, buparlisib, paxalisib, voxtalisib, zandelisib, dezapelisib, linperlisib, inavolisib, parsaclisib, eganelisib, nemiralisib, seletalisib, gedatolisib, leniolisib, tenalisib, pictilisib, bimiralisib, BBP-681, BGB-10188, MEN-1611, ASN-003, ACP-319, panobinostat, resminostat, abexinostat, romidepsin, belinostat, entinostat, quisinostat, pracinostat, tefinostat, mocetinostat, givinostat, dacinostat, ivaltinostat, domatinostat, fimepinostat, tinostamustine, Remetinostat, tucidinostat, ricolinostat, CXD-101, REC-2282, veltuzumab, rituximab, ublituximab, nogitecan, simmitecan, gimatecan, topotecan, cositecan, belotecan, govitecan, deruxtecan, AR-67, camsirubicin, Aldoxorubicin, vosaroxin, mitoxantrone, evofosfamide, amrubicin, sobuzoxane, epirubicin, F-14512, dacarbazine, temozolomide, nimustine, busulfan, procarbazine, melphalan, mitomycin C, daunorubicin, vincristine, vinblastine, vinorelbine, eribulin, trifluridine, docetaxel, cabazitaxel, avanbulin, fluorapacin, mertansine, carboplatin, nedaplatin, ixazomib, marizomib, carfilzomib and LXE-408.

55. An antitumor effect enhancer for at least one additional compound having an antitumor effect, at least one tautomer thereof, at least one solvate thereof, or at least one pharmaceutically acceptable salt thereof, selected from the group consisting of molecular targeted drugs and cytotoxic drugs,
the antitumor effect enhancer comprising a compound represented by formula (I) 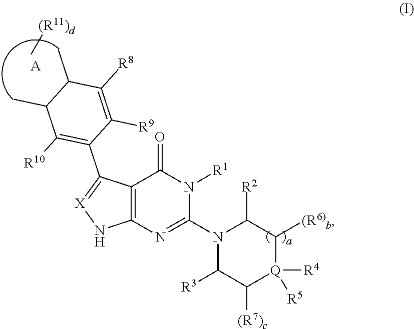
or a tautomer thereof, a solvate thereof, or a pharmaceutically acceptable salt thereof, wherein:
X is CH or N,
R¹ is —CH₃,
R² and R³ are independently selected from the group consisting of hydrogen and C₁₋₄alkyl,
Q is C or N,
wherein when Q is C, then either:
(i) R⁴ is amino, aminoC₁₋₄alkyl, or monoC₁₋₄alkylamino,
R⁵ is hydrogen, C₁₋₄alkyl, halogen, hydroxyC₁₋₄alkyl, C₁₋₄alkoxy, haloC₁₋₄alkyl, or C₁₋₄alkoxyC₁₋₄alkyl,
or
(ii) R⁴ and R⁵ together with Q form a four- to six-membered ring that can optionally contain 1 to 3 heteroatoms or groups independently selected from the group consisting of N, O, S, NH, C(O), and S(O)ₘ, and said ring formed by R⁴ and R⁵ can be unsubstituted or substituted with 1 to 4 groups independently selected from the group consisting of amino, halogen, haloC₁₋₄alkyl, hydroxyl, methoxy, methylamino, and C₁₋₄alkyl, and m is selected from the group consisting of 1 and 2, and
wherein when Q is N, then:
R⁴ is absent, and
R⁵ is hydrogen,
R⁶ and R⁷ are independently selected from the group consisting of halogen, C₁₋₄alkyl, hydroxyC₁₋₄alkyl, and hydroxyl, provided that when Q is N, then R⁶ or R⁷ is not halogen or hydroxyl,
or any two groups selected from the group consisting of R², R³, R⁶ and R⁷ together form a one- to three-membered bridge group selected from the group consisting of C₁₋₃alkylene, C₂₋₃alkenylene, methylene-NR^{q}-methylene and methylene-O-methylene, wherein the bridge group is optionally substituted with a group selected from the group consisting of C₁₋₄alkyl, hydroxyl, and halogen, and R^{q} is selected from the group consisting of hydrogen and C₁₋₄alkyl,
or R⁴ and R⁷ form a four- to six-membered ring containing a N atom,
or R⁵ and R⁷ form a three- to six-membered ring,
or R⁶ and R⁷ form a direct bond,
a is selected from the group consisting of 0, 1, and 2,
b is selected from the group consisting of 0, 1, and 2,
c is selected from the group consisting of 0, 1, and 2,
or Q is C, c is 2, R⁴ is hydrogen, and the two R⁷ join to form a 4 to 6-membered nitrogen containing ring,
Ring A is either:
(i) a five-membered nitrogen-containing heterocyclic ring wherein the heterocyclic ring optionally contains one or two additional heteroatoms selected from the group consisting of N, O, and S, or
(ii) a six-membered aromatic nitrogen-containing heterocyclic ring, wherein the heterocyclic ring optionally contains one or two additional heteroatoms selected from the group consisting of N, O, and S, or
(iii) a six-membered non-aromatic nitrogen-containing heterocyclic ring, wherein the heterocyclic ring optionally contains one or two additional heteroatoms selected from the group consisting of N and S,
R⁸ is selected from the group consisting of hydrogen, C₁₋₄alkyl, haloC₁₋₄alkyl, and halogen,
R⁹ is selected from the group consisting of hydrogen and halogen,
R¹⁰ is selected from the group consisting of haloC₁₋₄alkyl, C₁₋₄alkyl, halogen, hydrogen, and C₁₋₄alkoxy,
R¹¹ are independently selected from the group consisting of halogen, cyano, cyanoC₁₋₄alkyl, hydroxyl, oxo (═O), C₁₋₄alkyl optionally substituted with a five- or six-membered heterocyclic group containing 1 or 2 heteroatoms selected from the group consisting of O, N, and S, haloC₁₋₄alkyl, C₁₋₄alkoxy, hydroxylC₁₋₄alkyl, C₁₋₄alkoxyC₁₋₄alkyl, C₁₋₄alkylsulfone, amino, monoC₁₋₄alkylamino, diC₁₋₄alkylamino, aminoC₁₋₄alkyl, —C₁₋₄alkylene-C(═O)NH_{(2-q)}(C₁₋₆alkyl)_{q}), —C₁₋₄alkylene-NHC(═O)C₁₋₆alkyl, sulfonamide, sulfonamideC₁₋₄alkyl, 3 to 6-membered cycloalkyl, C₁₋₄alkyl substituted with 3 to 6-membered cycloalkyl, a five- or six-membered unsaturated heterocyclic group containing 1, 2, 3, or 4 heteroatoms selected from the group consisting of O, N, and S, and an optionally substituted four- to six-membered saturated heterocyclic group containing 1 or 2 heteroatoms selected from the group consisting of O, N, and S where the optional substituent is selected from C₁₋₄alkyl,
q is selected from the group consisting of 0, 1 and 2, and
d is selected from the group consisting of 0, 1 and 2,
as an active ingredient.

56. An antitumor agent comprising a compound represented by formula (I) 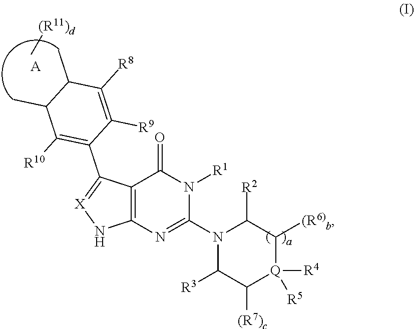
or a tautomer thereof, a solvate thereof, or a pharmaceutically acceptable salt thereof, wherein:
X is CH or N,
R¹ is —CH₃,
R² and R³ are independently selected from the group consisting of hydrogen and C₁₋₄alkyl,
Q is C or N,
wherein when Q is C, then either:
(i) R⁴ is amino, aminoC₁₋₄alkyl, or monoC₁₋₄alkylamino, R⁵ is hydrogen, C₁₋₄alkyl, halogen, hydroxyC₁₋₄alkyl, C₁₋₄alkoxy, haloC₁₋₄alkyl, or C₁₋₄alkoxyC₁₋₄alkyl,
or
(ii) R⁴ and R⁵ together with Q form a four- to six-membered ring that can optionally contain 1 to 3 heteroatoms or groups independently selected from the group consisting of N, O, S, NH, C(O), and S(O)ₘ, and said ring formed by R⁴ and R⁵ can be unsubstituted or substituted with 1 to 4 groups independently selected from the group consisting of amino, halogen, haloC₁₋₄alkyl, hydroxyl, methoxy, methylamino, and C₁₋₄alkyl, and m is selected from the group consisting of 1 and 2, and
wherein when Q is N, then:
R⁴ is absent, and
R⁵ is hydrogen,
R⁶ and R⁷ are independently selected from the group consisting of halogen, C₁₋₄alkyl, hydroxyC₁₋₄alkyl, and hydroxyl, provided that when Q is N, then R⁶ or R⁷ is not halogen or hydroxyl,
or any two groups selected from the group consisting of R², R³, R⁶, and R⁷ together form a one- to three-membered bridge group selected from the group consisting of C₁₋₃alkylene, C₂₋₃alkenylene, methylene-NR^{q}-methylene, and methylene-O-methylene, wherein the bridge group is optionally substituted with a group selected from the group consisting of C₁₋₄alkyl, hydroxyl and halogen, and R^{q} is selected from the group consisting of hydrogen and C₁₋₄alkyl,
or R⁴ and R⁷ form a four- to six-membered ring containing a N atom,
or R⁵ and R⁷ form a three- to six-membered ring,
or R⁶ and R⁷ form a direct bond,
a is selected from the group consisting of 0, 1, and 2,
b is selected from the group consisting of 0, 1, and 2,
c is selected from the group consisting of 0, 1, and 2,
or Q is C, c is 2, R⁴ is hydrogen, and the two R⁷ join to form a 4 to 6-membered nitrogen containing ring,
Ring A is either:
(i) a five-membered nitrogen-containing heterocyclic ring wherein the heterocyclic ring optionally contains one or two additional heteroatoms selected from the group consisting of N, O, and S, or
(ii) a six-membered aromatic nitrogen-containing heterocyclic ring, wherein the heterocyclic ring optionally contains one or two additional heteroatoms selected from the group consisting of N, O, and S, or
(iii) a six-membered non-aromatic nitrogen-containing heterocyclic ring, wherein the heterocyclic ring optionally contains one or two additional heteroatoms selected from the group consisting of N and S,
R⁸ is selected from the group consisting of hydrogen, C₁₋₄alkyl, haloC₁₋₄alkyl, and halogen,
R⁹ is selected from the group consisting of hydrogen and halogen,
R¹⁰ is selected from the group consisting of haloC₁₋₄alkyl, C₁₋₄alkyl, halogen, hydrogen, and C₁₋₄alkoxy,
R¹¹ are independently selected from the group consisting of halogen, cyano, cyanoC₁₋₄alkyl, hydroxyl, oxo (═O), C₁₋₄alkyl optionally substituted with a five- or six-membered heterocyclic group containing 1 or 2 heteroatoms selected from the group consisting of O, N, and S, haloC₁₋₄alkyl, C₁₋₄alkoxy, hydroxylC₁₋₄alkyl, C₁₋₄alkoxyC₁₋₄alkyl, C₁₋₄alkylsulfone, amino, monoC₁₋₄alkylamino, diC₁₋₄alkylamino, aminoC₁₋₄alkyl, —C₁₋₄alkylene-C(═O)NH_{(2-q)}(C₁₋₆alkyl)_{q}), —C₁₋₄alkylene-NHC(═O)C₁₋₆alkyl, sulfonamide, sulfonamideC₁₋₄alkyl, 3 to 6-membered cycloalkyl, C₁₋₄alkyl substituted with 3 to 6-membered cycloalkyl, a five- or six-membered unsaturated heterocyclic group containing 1, 2, 3, or 4 heteroatoms selected from the group consisting of O, N, and S, and an optionally substituted four- to six-membered saturated heterocyclic group containing 1 or 2 heteroatoms selected from the group consisting of O, N, and S where the optional substituent is selected from C₁₋₄alkyl,
q is selected from the group consisting of 0, 1, and 2, and
d is selected from the group consisting of 0, 1, and 2,
wherein the antitumor agent is concomitantly used with at least one additional compound having an antitumor effect, at least one tautomer thereof, at least one solvate thereof, or at least one pharmaceutically acceptable salt thereof, selected from the group consisting of molecular targeted drugs and cytotoxic drugs.

57. A method of treating a tumor comprising administering a compound represented by formula (I) 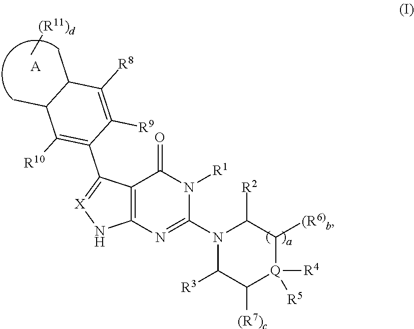
or a tautomer thereof, a solvate thereof, or a pharmaceutically acceptable salt thereof, wherein:
X is CH or N,
R¹ is —CH₃,
R² and R³ are independently selected from the group consisting of hydrogen and C₁₋₄alkyl,
Q is C or N,
wherein when Q is C, then either:
(i) R⁴ is amino, aminoC₁₋₄alkyl or monoC₁₋₄alkylamino,
R⁵ is hydrogen, C₁₋₄alkyl, halogen, hydroxyC₁₋₄alkyl, C₁₋₄alkoxy, haloC₁₋₄alkyl, or C₁₋₄alkoxyC₁₋₄alkyl,
or
(ii) R⁴ and R⁵ together with Q form a four- to six-membered ring that can optionally contain 1 to 3 heteroatoms or groups independently selected from the group consisting of N, O, S, NH, C(O), and S(O)ₘ, and said ring formed by R⁴ and R⁵ can be unsubstituted or substituted with 1 to 4 groups independently selected from the group consisting of amino, halogen, haloC₁₋₄alkyl, hydroxyl, methoxy, methylamino, and C₁₋₄alkyl, and m is selected from the group consisting of 1 and 2, and
wherein when Q is N, then:
R⁴ is absent, and
R⁵ is hydrogen,
R⁶ and R⁷ are independently selected from the group consisting of halogen, C₁₋₄alkyl, hydroxyC₁₋₄alkyl, and hydroxyl, provided that when Q is N, then R⁶ or R⁷ is not halogen or hydroxyl,
or any two groups selected from the group consisting of R², R³, R⁶, and R⁷ together form a one- to three-membered bridge group selected from the group consisting of C₁₋₃alkylene, C₂₋₃alkenylene, methylene-NR^{q}-methylene, and methylene-O-methylene, wherein the bridge group is optionally substituted with a group selected from the group consisting of C₁₋₄alkyl, hydroxyl, and halogen, and R^{q} is selected from the group consisting of hydrogen and C₁₋₄alkyl,
or R⁴ and R⁷ form a four- to six-membered ring containing a N atom,
or R⁵ and R⁷ form a three- to six-membered ring,
or R⁶ and R⁷ form a direct bond,
a is selected from the group consisting of 0, 1, and 2,
b is selected from the group consisting of 0, 1, and 2,
c is selected from the group consisting of 0, 1, and 2,
or Q is C, c is 2, R⁴ is hydrogen, and the two R⁷ join to form a 4 to 6-membered nitrogen containing ring,
Ring A is either:
(i) a five-membered nitrogen-containing heterocyclic ring wherein the heterocyclic ring optionally contains one or two additional heteroatoms selected from the group consisting of N, O, and S, or
(ii) a six-membered aromatic nitrogen-containing heterocyclic ring, wherein the heterocyclic ring optionally contains one or two additional heteroatoms selected from the group consisting of N, O, and S, or
(iii) a six-membered non-aromatic nitrogen-containing heterocyclic ring, wherein the heterocyclic ring optionally contains one or two additional heteroatoms selected from the group consisting of N and S,
R⁸ is selected from the group consisting of hydrogen, C₁₋₄alkyl, haloC₁₋₄alkyl, and halogen,
R⁹ is selected from the group consisting of hydrogen and halogen,
R¹⁰ is selected from the group consisting of haloC₁₋₄alkyl, C₁₋₄alkyl, halogen, hydrogen, and C₁₋₄alkoxy,
R¹¹ are independently selected from the group consisting of halogen, cyano, cyanoC₁₋₄alkyl, hydroxyl, oxo (═O), C₁₋₄alkyl optionally substituted with a five- or six-membered heterocyclic group containing 1 or 2 heteroatoms selected from the group consisting of O, N, and S, haloC₁₋₄alkyl, C₁₋₄alkoxy, hydroxylC₁₋₄alkyl, C₁₋₄alkoxyC₁₋₄alkyl, C₁₋₄alkylsulfone, amino, monoC₁₋₄alkylamino, diC₁₋₄alkylamino, aminoC₁₋₄alkyl, —C₁₋₄alkylene-C(═O)NH_{(2-q)}(C₁₋₆alkyl)_{q}), —C₁₋₄alkylene-NHC(═O)C₁₋₆alkyl, sulfonamide, sulfonamideC₁₋₄alkyl, 3 to 6-membered cycloalkyl, C₁₋₄alkyl substituted with 3 to 6-membered cycloalkyl, a five- or six-membered unsaturated heterocyclic group containing 1, 2, 3, or 4 heteroatoms selected from the group consisting of O, N, and S, and an optionally substituted four- to six-membered saturated heterocyclic group containing 1 or 2 heteroatoms selected from the group consisting of O, N, and S where the optional substituent is selected from C₁₋₄alkyl,
q is selected from the group consisting of 0, 1, and 2, and
d is selected from the group consisting of 0, 1, and 2, and at least one additional compound having an antitumor effect, at least one tautomer thereof, at least one solvate thereof, or at least one pharmaceutically acceptable salt thereof, selected from the group consisting of molecular targeted drugs and cytotoxic drugs.

58. The method for treating a tumor according to claim 57, wherein the compound represented by formula (I), the tautomer thereof, the solvate thereof, or the pharmaceutically acceptable salt thereof is administered before, simultaneously with, or after administration of the at least one additional compound having an antitumor effect, the at least one tautomer thereof, the at least one solvate thereof, or the at least one pharmaceutically acceptable salt thereof.

59. The method for treating a tumor according to claim 57, wherein the compound represented by formula (I) is selected from the group consisting of 2-((1R,2R,4S)-2-amino-7-azabicyclo[2.2.1]heptan-7-yl)-5-(3,4-dichloro-2-methyl-2H-indazol-5-yl)-3-methyl-3,7-dihydro-4H-pyrrolo[2,3-d]pyrimidin-4-one, 6-((1R,4R,7R)-7-amino-2-azabicyclo[2.2.1]heptan-2-yl)-3-(3,4-dichloro-2-methyl-2H-indazol-5-yl)-5-methyl-1,5-dihydro-4H-pyrazolo[3,4-d]pyrimidin-4-one, 2-((1R,4R,7R)-7-amino-2-azabicyclo[2.2.1]heptan-2-yl)-5-(4-chloro-2-ethyl-2H-indazol-5-yl)-3-methyl-3,7-dihydro-4H-pyrrolo[2,3-d]pyrimidin-4-one, 2-((1R,4R,7R)-7-amino-2-azabicyclo[2.2.1]heptan-2-yl)-5-(4-chloro-2-methyl-2H-indazol-5-yl)-3-methyl-3,7-dihydro-4H-pyrrolo[2,3-d]pyrimidin-4-one, 2-((1R,4R,7R)-7-amino-2-azabicyclo[2.2.1]heptan-2-yl)-5-(3,4-dichloro-2-methyl-2H-indazol-5-yl)-3-methyl-3,7-dihydro-4H-pyrrolo[2,3-d]pyrimidin-4-one, 2-((1R,2R,4S)-2-amino-7-azabicyclo[2.2.1]heptan-7-yl)-5-(3-chloro-4-fluoro-2-methyl-2H-indazol-5-yl)-3-methyl-3,7-dihydro-4H-pyrrolo[2,3-d]pyrimidin-4-one, and 2-((1R,2R,4S)-2-amino-7-azabicyclo[2.2.1]heptan-7-yl)-5-(4-chloro-2-ethyl-2H-indazol-5-yl)-3-methyl-3,7-dihydro-4H-pyrrolo[2,3-d]pyrimidin-4-one.

60. The method for treating a tumor according to claim 57, wherein the compound represented by formula (I) is 2-((1R,2R,4S)-2-amino-7-azabicyclo[2.2.1]heptan-7-yl)-5-(3,4-dichloro-2-methyl-2H-indazol-5-yl)-3-methyl-3,7-dihydro-4H-pyrrolo[2,3-d]pyrimidin-4-one.

61. The method for treating a tumor according to claim 57, wherein the at least one additional compound having an antitumor effect, the at least one tautomer thereof, the at least one solvate thereof, or the at least one pharmaceutically acceptable salt thereof is selected from the group consisting of a tyrosine kinase inhibitor, a RAS-MAPK pathway inhibitor, a PI3K pathway inhibitor, a BCL2 inhibitor, a CDK4/6 inhibitor, an HDAC inhibitor, a topoisomerase inhibitor (a topoisomerase I inhibitor, and topoisomerase II inhibitor), an alkylating agent, an anthracycline antibiotic, an alkaloid, an anti-metabolite, an anti-microtubule agent, a platinum-containing drug, a proteasome inhibitor, and a thalidomide analog drug.

62. The method for treating a tumor according to claim 57, wherein the molecular targeted drug is selected from the group consisting of an AKT inhibitor, ALK inhibitor, Bcl2 inhibitor, BCR-ABL inhibitor, BRAF inhibitor, CDK4/6 inhibitor, CDK inhibitor, EGFR inhibitor, Erk1/2 inhibitor, FGFR inhibitor, FLT3 inhibitor, HER2 inhibitor, MEK inhibitor, Multi-kinase inhibitor, PI3K inhibitor, RAF inhibitor, HDAC inhibitor, topoisomerase I inhibitor, topoisomerase II inhibitor, alkylating agent, anthracycline antibiotics, alkaloid, anti-metabolite, anti-microtubule agent, platinum-containing drug, and proteasome inhibitor.

63. The method for treating a tumor according to claim 57, wherein the molecular targeted drug is selected from the group consisting of cetuximab, MK-2206, alectinib, crizotinib, venetoclax, imatinib, dasatinib, ponatinib, dabrafenib, vemurafenib, sorafenib, palbociclib, abemaciclib, osimertinib, gefitinib, erlotinib, afatinib, brigatinib, ulixertinib, (2R)-2-(6-{5-chloro-2-[(oxan-4-yl)amino]pyrimidin-4-yl}-1-oxo-2,3-dihydro-1H-isoindol-2-yl)-N-[(1S)-1-(3-fluoro-5-methoxyphenyl)-2-hydroxyethyl]propanamide or a salt thereof, erdafitinib, giltertinib, lapatinib, neratinib, trametinib, cobimetinib, binimetinib, regorafenib, sunitinib, nintedanib, anlotinib, vandetanib, lenvatinib, alpelisib and idelalisib/CAL-101, vorinostat (SAHA), irinotecan (SN-38), etoposide, cyclophosphamide, doxorubicin, gemcitabine, pemetrexed, 5-FU, FdUrd, FTD, paclitaxel, cisplatin, oxaliplatin, bortezomib, afuresertib, trans-3-amino-1-methyl-3-(4-(3-phenyl-5H-imidazo[1,2-c]pyrido[3,4-e][1,3]oxazin-2-yl)phenyl)cyclobutanol, capivasertib, ipatasertib, triciribine, miransertib, lorlatinib, ceritinib, repotrectinib, ensartinib, alkotinib, WX-0593, SAF-189s, CT-707, TQ-B3101, sabutoclax, apogossypol, obatoclax, navitoclax, APG-2575, APG-1252, asciminib, olverembatinib, encorafenib, lifirafenib, LXH-254, ribociclib, lerociclib, trilaciclib, alvocidib, GLR-2007, SHR-6390, XZP-3287, BPI-1178, PF-06873600, NUV-422, FCN-437, seliciclib, mevociclib, milciclib, fadraciclib, zotiraciclib, dinaciclib, samuraciclib, voruciclib, FIT-039, PF-07104091, BEY-1107, panitumumab, sutetinib, allitinib, epitinib, xiliertinib, rociletinib, dacomitinib, simotinib, olmutinib, yinlitinib, mefatinib, alflutinib, almonertinib, icotinib, naquotinib, poziotinib, epertinib, sapitinib, cipatinib, tarloxotinib, pyrotinib, pirotinib, lazertinib, varlitinib, tesevatinib, canertinib, mobocertinib, duligotuzumab, olafertinib, zorifertinib, pelitinib, DZD-9008, ASK-120067, BPI-7711, QLNC-120, ametumumab, imgatuzumab, amivantamab, seribantumab, nimotuzumab, serclutamab, depatuxizumab, tomuzotuximab, SCT-200, ravoxertinib, LY3214996, MK-8353, LTT462, HH-2710, infigratinib, pemigatinib, orantinib, derazantinib, roblitinib, rogaratinib, zoligratinib, E-7090, AZD-4547, ODM-203, ICP-192, HMPL-453, bemarituzumab, quizartinib, crenolanib, flysyn, mivavotinib, PHI-101, MEN-1703, FF-10101, HM-43239, E-6201, ENMD-2076, larotinib, tucatinib (irbinitinib), BDTX-189, trastuzumab, pertuzumab, zanidatamab, zenocutuzumab, margetuximab, KN-026, BAT-8001, TAA-013, KL-A166, selumetinib, refametinib, mirdametinib, pimasertib, HL-085, NFX-179, nilotinib, dovitinib, axitinib, vatalinib, pazopanib, avapritinib, famitinib, catequentinib, necuparanib, surufatinib, lucitanib, midostaurin, vorolanib, bevasiranib, bevacizumab, ranibizumab, vanucizumab, navicixizumab, ramucirumab, BAT-5906, VGX-100, CSL-346, duvelisib, copanlisib, buparlisib, paxalisib, voxtalisib, zandelisib, dezapelisib, linperlisib, inavolisib, parsaclisib, eganelisib, nemiralisib, seletalisib, gedatolisib, leniolisib, tenalisib, pictilisib, bimiralisib, BBP-681, BGB-10188, MEN-1611, ASN-003, ACP-319, panobinostat, resminostat, abexinostat, romidepsin, belinostat, entinostat, quisinostat, pracinostat, tefinostat, mocetinostat, givinostat, dacinostat, ivaltinostat, domatinostat, fimepinostat, tinostamustine, Remetinostat, tucidinostat, ricolinostat, CXD-101, REC-2282, veltuzumab, rituximab, ublituximab, nogitecan, simmitecan, gimatecan, topotecan, cositecan, belotecan, govitecan, deruxtecan, AR-67, camsirubicin, Aldoxorubicin, vosaroxin, mitoxantrone, evofosfamide, amrubicin, sobuzoxane, epirubicin, F-14512, dacarbazine, temozolomide, nimustine, busulfan, procarbazine, melphalan, mitomycin C, daunorubicin, vincristine, vinblastine, vinorelbine, eribulin, trifluridine, docetaxel, cabazitaxel, avanbulin, fluorapacin, mertansine, carboplatin, nedaplatin, ixazomib, marizomib, carfilzomib and LXE-408.

64. The method for treating a tumor according to claim 57, wherein the malignant tumor is selected from the group consisting of epithelial cancer (respiratory cancer, gastrointestinal cancer, genital cancer, cancer of the secretory system, breast cancer, etc.), mesothelioma, sarcoma, hematopoietic tumor, tumors of the central nervous system, retinoblastoma, and tumors of the peripheral nervous system.

65. A kit for malignant tumor treatment comprising a compound represented by formula (I) 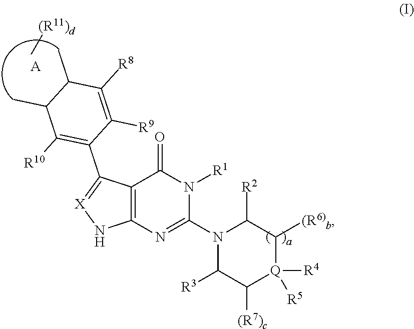
or a tautomer thereof, a solvate thereof, or a pharmaceutically acceptable salt thereof, wherein:
X is CH or N,
R¹ is —CH₃,
R² and R³ are independently selected from the group consisting of hydrogen and C₁₋₄alkyl,
Q is C or N,
wherein when Q is C, then either:
(i) R⁴ is amino, aminoC₁₋₄alkyl, or monoC₁₋₄alkylamino,
R⁵ is hydrogen, C₁₋₄alkyl, halogen, hydroxyC₁₋₄alkyl, C₁₋₄alkoxy, haloC₁₋₄alkyl, or C₁₋₄alkoxyC₁₋₄alkyl,
or
(ii) R⁴ and R⁵ together with Q form a four- to six-membered ring that can optionally contain 1 to 3 heteroatoms or groups independently selected from the group consisting of N, O, S, NH, C(O), and S(O)ₘ, and said ring formed by R⁴ and R⁵ can be unsubstituted or substituted with 1 to 4 groups independently selected from the group consisting of amino, halogen, haloC₁₋₄alkyl, hydroxyl, methoxy, methylamino, and C₁₋₄alkyl, and m is selected from the group consisting of 1 and 2, and
wherein when Q is N, then:
R⁴ is absent, and
R⁵ is hydrogen,
R⁶ and R⁷ are independently selected from the group consisting of halogen, C₁₋₄alkyl, hydroxyC₁₋₄alkyl, and hydroxyl, provided that when Q is N, then R⁶ or R⁷ is not halogen or hydroxyl,
or any two groups selected from the group consisting of R², R³, R⁶, and R⁷ together form a one- to three-membered bridge group selected from the group consisting of C₁₋₃alkylene, C₂₋₃alkenylene, methylene-NR^{q}-methylene, and methylene-O-methylene, wherein the bridge group is optionally substituted with a group selected from the group consisting of C₁₋₄alkyl, hydroxyl, and halogen, and R^{q} is selected from the group consisting of hydrogen and C₁₋₄alkyl,
or R⁴ and R⁷ form a four- to six-membered ring containing a N atom,
or R⁵ and R⁷ form a three- to six-membered ring,
or R⁶ and R⁷ form a direct bond,
a is selected from the group consisting of 0, 1, and 2,
b is selected from the group consisting of 0, 1, and 2,
c is selected from the group consisting of 0, 1, and 2,
or Q is C, c is 2, R⁴ is hydrogen, and the two R⁷ join to form a 4 to 6-membered nitrogen containing ring,
Ring A is either:
(i) a five-membered nitrogen-containing heterocyclic ring wherein the heterocyclic ring optionally contains one or two additional heteroatoms selected from the group consisting of N, O, and S, or
(ii) a six-membered aromatic nitrogen-containing heterocyclic ring, wherein the heterocyclic ring optionally contains one or two additional heteroatoms selected from the group consisting of N, O, and S, or
(iii) a six-membered non-aromatic nitrogen-containing heterocyclic ring, wherein the heterocyclic ring optionally contains one or two additional heteroatoms selected from the group consisting of N and S,
R⁸ is selected from the group consisting of hydrogen, C₁₋₄alkyl, haloC₁₋₄alkyl and halogen,
R⁹ is selected from the group consisting of hydrogen and halogen,
R¹⁰ is selected from the group consisting of haloC₁₋₄alkyl, C₁₋₄alkyl, halogen, hydrogen, and C₁₋₄alkoxy,
R¹¹ are independently selected from the group consisting of halogen, cyano, cyanoC₁₋₄alkyl, hydroxyl, oxo (═O), C₁₋₄alkyl optionally substituted with a five- or six-membered heterocyclic group containing 1 or 2 heteroatoms selected from the group consisting of O, N, and S, haloC₁₋₄alkyl, C₁₋₄alkoxy, hydroxylC₁₋₄alkyl, C₁₋₄alkoxyC₁₋₄alkyl, C₁₋₄alkylsulfone, amino, monoC₁₋₄alkylamino, diC₁₋₄alkylamino, aminoC₁₋₄alkyl, —C₁₋₄alkylene-C(═O)NH_{(2-q)}(C₁₋₆alkyl)_{q}), —C₁₋₄alkylene-NHC(═O)C₁₋₆alkyl, sulfonamide, sulfonamideC₁₋₄alkyl, 3 to 6-membered cycloalkyl, C₁₋₄alkyl substituted with 3 to 6-membered cycloalkyl, a five- or six-membered unsaturated heterocyclic group containing 1, 2, 3 or 4 heteroatoms selected from the group consisting of O, N, and S, and an optionally substituted four- to six-membered saturated heterocyclic group containing 1 or 2 heteroatoms selected from the group consisting of O, N, and S where the optional substituent is selected from C₁₋₄alkyl,
q is selected from the group consisting of 0, 1, and 2, and
d is selected from the group consisting of 0, 1, and 2
and at least one additional compound having an antitumor effect, at least one tautomer thereof, at least one solvate thereof, or at least one pharmaceutically acceptable salt thereof, selected from the group consisting of molecular targeted drugs and cytotoxic drugs.
